(19) United States
(12) Patent Application Publication
(10) Pub. No.: US 2025/0243170 A1
(43) Pub. Date: Jul. 31, 2025

(54) REMIBRUTINIB DRUG SUBSTANCE AND DRUG PRODUCT SUBSTANTIALLY FREE OF NITROSAMINE IMPURITY

(71) Applicant: NOVARTIS AG, Basel (CH)

(72) Inventors: Jožko CESAR, Ljubljana (SI); Christian Roger Claude FLEURY, Delémont (CH); Thorsten KLANG, Möhlin (CH); Maša LOGAR, Ilirska Bistrica (SI); Lucie LOVELLE, Basel (CH); Doris Walli PUPOWICZ, Dornach (CH); Peter ROBIC, Hrastnik (SI); Ana SLAKAN, Šentrupert (SI)

(21) Appl. No.: 19/032,740

(22) Filed: Jan. 21, 2025

Related U.S. Application Data
(60) Provisional application No. 63/727,876, filed on Dec. 4, 2024.
(60) Provisional application No. 63/625,341, filed on Jan. 26, 2024.

(51) Int. Cl. C07D 239/47; A61K 31/505

(52) U.S. Cl. CPC C07D 239/47; A61K 31/505

(57) ABSTRACT

This invention relates to N-(3-(6-Amino-5-(2-(N-methylacrylamido)ethoxy)pyrimidin-4-yl)-5-fluoro-2-methylphenyl)-4-cyclopropyl-2-fluorobenzamide drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide and novel processes of preparation thereof. The invention further relates to pharmaceutical composition comprising N-(3-(6-Amino-5-(2-(N-methylacrylamido)ethoxy)pyrimidin-4-yl)-5-fluoro-2-methylphenyl)-4-cyclopropyl-2-fluorobenzamide, wherein the composition is substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide. Pharmaceutical products containing said drug substance and compositions are also disclosed, as well as methods for preparing said drug substance, pharmaceutical compositions and products.

### FIELD OF THE INVENTION

The present invention relates to N-(3-(6-Amino-5-(2-(N-methylacrylamido)ethoxy)pyrimidin-4-yl)-5-fluoro-2-methylphenyl)-4-cyclopropyl-2-fluorobenzamide drug substance substantially free of nitrosamine impurities as well as new methods of preparation thereof. The invention further relates to a pharmaceutical composition, comprising N-(3-(6-Amino-5-(2-(N-methylacrylamido)ethoxy)pyrimidin-4-yl)-5-fluoro-2-methylphenyl)-4-cyclopropyl-2-fluorobenzamide, wherein said composition is substantially free of nitrosamine impurities. The invention also relates to methods for preparing said drug substance, pharmaceutical composition and products thereof.

### BACKGROUND

Nitrosamines are organic compounds containing the —NO functional group, associated with an amine group. Certain members of this family of compounds have been identified as potentially carcinogenic to humans. They can form in medicines and pharmaceutical products during manufacturing, storage or even use.

Of major concern is that long-term exposure to high levels of nitrosamines may increase the risk of cancer. Some types of nitrosamines are classified as known or probable human carcinogens by regulatory agencies such as the International Agency for Research on Cancer (IARC). To ensure patient safety, regulatory authorities, such as the Food and Drug Administration (FDA) in the United States and the European Medicines Agency (EMA), impose strict limits on the amount of nitrosamines allowed in pharmaceutical products. Drug manufacturers must implement rigorous quality controls to minimize the formation of these unwanted compounds and ensure compliance with established safety standards. Continuous monitoring of the quality of medicines is therefore crucial to prevent any potential risks to patient health.

N-(3-(6-Amino-5-(2-(N-methylacrylamido)ethoxy)pyrimidin-4-yl)-5-fluoro-2-methylphenyl)-4-cyclopropyl-2-fluorobenzamide, (IUPAC name: N-[3-(6-Amino-5-{2-[methyl(prop-2-enoyl)amino]ethoxy}pyrimidin-4-yl)-5-fluoro-2-methylphenyl]-4-cyclopropyl-2-fluorobenzamide), also known as remibrutinib, is a highly potent and selective oral Bruton's tyrosine kinase (BTK) inhibitor:

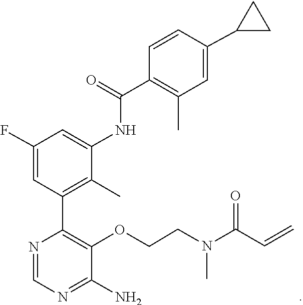 [SMILES: C=CC(=O)N(C)CCOc1c(N)ncnc1-c1cc(F)cc(NC(=O)c2ccc(C3CC3)cc2C)c1C]

Remibrutinib (also known as “LOU064”) was first disclosed in WO2015/079417, filed Nov. 28, 2014, in Example 6. WO2015/079417 is incorporated by reference in its entirety. “LOU064” and “remibrutinib” will be used interchangeably. In WO2015/079417, Example 6(2), remibrutinib is prepared by cross-coupling of “INT 5” with “INT 8”, to give “INT 9”:

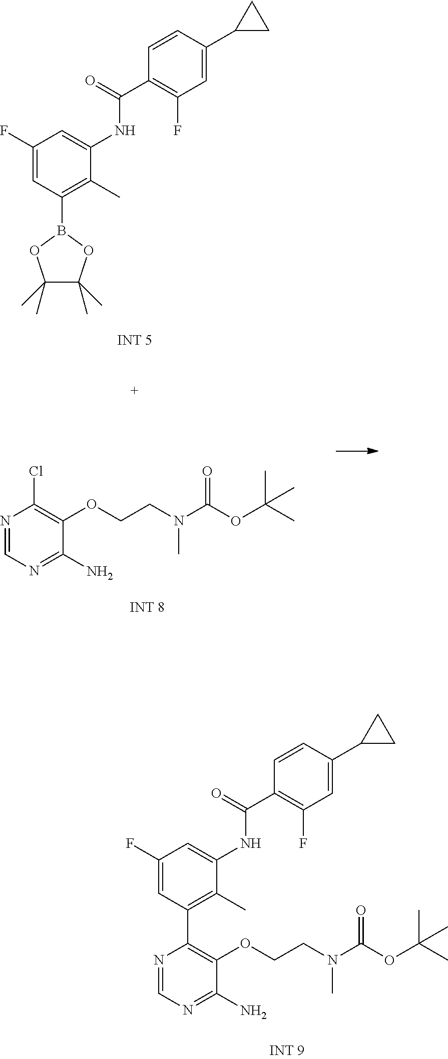 [SMILES: Cc1c(NC(=O)c2ccc(C3CC3)cc2F)cc(F)cc1B1OC(C)(C)C(C)(C)O1 ; CN(CCOc1c(N)ncnc1Cl)C(=O)OC(C)(C)C ; Cc1c(NC(=O)c2ccc(C3CC3)cc2F)cc(F)cc1-c1ncnc(N)c1OCCN(C)C(=O)OC(C)(C)C]

INT 9 is then deprotected with TFA (Example 6(3)), reacted with acrylic acid, and purified to give remibrutinib (Example 6(4)). The preparation of INT 5 is described in Example 1(5) of WO2015/079417. INT 5 is prepared by the amide coupling of INT 3 and INT 4:

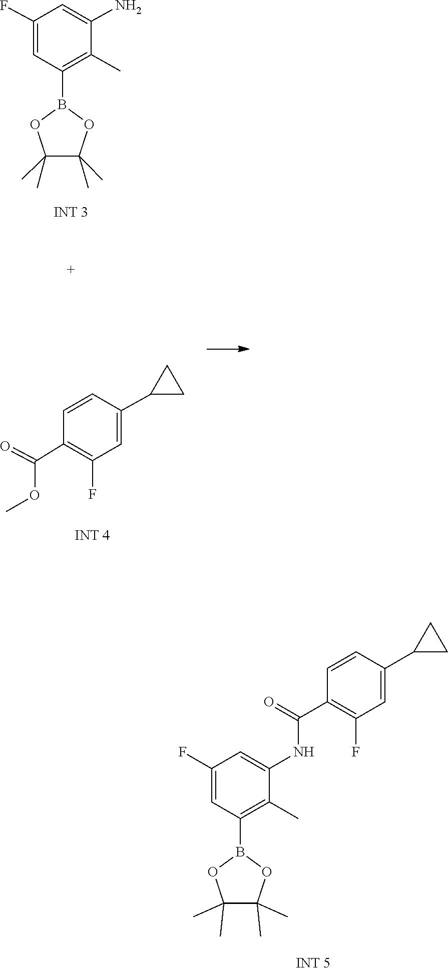 [SMILES: Cc1c(N)cc(F)cc1B1OC(C)(C)C(C)(C)O1 ; COC(=O)c1ccc(C2CC2)cc1F ; Cc1c(NC(=O)c2ccc(C3CC3)cc2F)cc(F)cc1B1OC(C)(C)C(C)(C)O1]

However, it has been discovered that INT 3 (5-fluoro-2-methyl-3-(4,4,5,5-tetramethyl-1,3,2-dioxaborolan-2-yl)aniline), is a compound with mutagenic potential and is therefore an undesirable intermediate in the synthesis of a medicinal product. The genotoxicity of INT 3 is reported for the first time in the present application.

Therefore, new synthetic routes for the preparation of remibrutinib avoiding the use of 5-fluoro-2-methyl-3-(4,4,5,5-tetramethyl-1,3,2-dioxaborolan-2-yl)aniline have been disclosed in PCT/IB2023/059664 (Attorney reference number (PAT059209-WO-PCT)). PCT/IB2023/059664 is incorporated by reference herein in its entirety.

It has now been discovered that the process disclosed in PCT/IB2023/059664 provides remibrutinib with the presence of a nitrosamine impurity at a level of about 1.6 ppm. The nitrosamine impurity is N-(3-(6-amino-5-(2(methyl)(nitroso)amino)ethoxy)pyrimidin-4-yl)-5-fluoro-2-methylphenyl)-4-cyclopropyl-2-fluorobenzamide (IUPAC name: N-[3-(6-Amino-5-{2-[methyl(nitroso)amino]ethoxy}pyrimidin-4-yl)-5-fluoro-2-methylphenyl]-4-cyclopropyl-2-fluorobenzamide) and has the following structure:

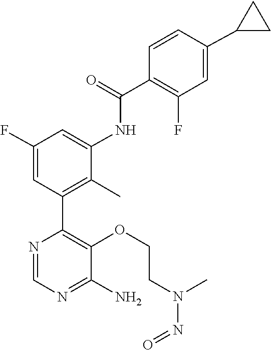 [SMILES: Cc1c(NC(=O)c2ccc(C3CC3)cc2F)cc(F)cc1-c1ncnc(N)c1OCCN(C)N=O]

which also exists in another stable form:

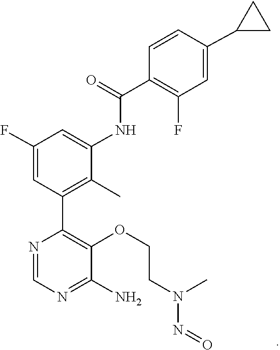 [SMILES: Cc1c(NC(=O)c2ccc(C3CC3)cc2F)cc(F)cc1-c1ncnc(N)c1OCCN(C)N=O]

It has been observed that such a nitrosamine impurity is formed during the last 2 steps of the process:

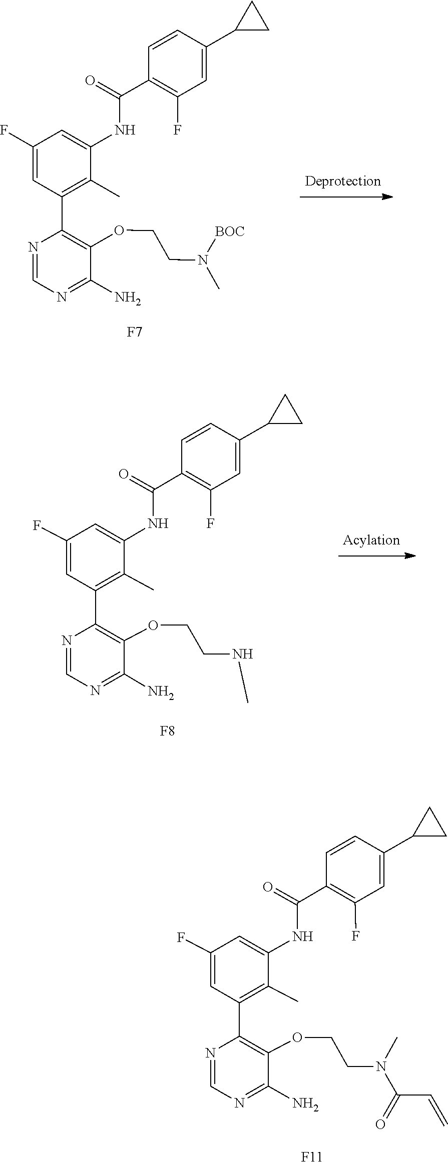

It has now further been established that N-(3-(6-amino-5-(2(methyl)(nitroso)amino)ethoxy)pyrimidin-4-yl)-5-fluoro-2-methylphenyl)-4-cyclopropyl-2-fluorobenzamide is positive in an enhanced Ames Test (EAT) and is therefore considered to be potentially mutagenic under sensitive metabolic conditions by health authorities. Even though the in vivo relevance of such positive EAT has not yet been established, it would be preferable to provide a drug substance free or substantially free of a nitrosamine impurity which provides a positive EAT result, e.g. free of N-(3-(6-amino-5-(2(methyl)(nitroso)amino)ethoxy)pyrimidin-4-yl)-5-fluoro-2-methylphenyl)-4-cyclopropyl-2-fluorobenzamide. The presence of a nitrosamine impurity in remibrutinib drug substance and its mutagenicity in EAT is first reported in the present application.

For example, it is therefore the object of this invention to provide remibrutinib drug substance substantially free of nitrosamine impurities, for example, while avoiding the use of 5-fluoro-2-methyl-3-(4,4,5,5-tetramethyl-1,3,2-dioxaborolan-2-yl)aniline intermediate. It is further the object of this invention to provide a pharmaceutical composition comprising remibrutinib or a pharmaceutically acceptable salt thereof and one or more excipients, and wherein the composition is substantially free of nitrosamine impurities, e.g. substantially free of N-(3-(6-amino-5-(2(methyl)(nitroso)amino)ethoxy)pyrimidin-4-yl)-5-fluoro-2-methylphenyl)-4-cyclopropyl-2-fluorobenzamide.

### SUMMARY OF THE INVENTION

The invention relates to reducing the amount of nitrosamine impurities, e.g. nitrosamine impurities which are positive in an EAT, e.g. reducing the amount of N-(3-(6-amino-5-(2(methyl)(nitroso)amino)ethoxy)pyrimidin-4-yl)-5-fluoro-2-methylphenyl)-4-cyclopropyl-2-fluorobenzamide in the LOU064 drug substance to certain levels, for example, where the content of nitrosamine in LOU064 drug substance is being less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb.

All of these nitrosamine impurity levels are specified relative to the total amount of the LOU064 drug substance.

In another aspect, the invention relates to remibrutinib drug substance or drug product that is substantially free from an impurity that produces a positive EAT, e.g., substantially free from a nitrosamine impurity; e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide.

In another aspect, the invention relates to remibrutinib drug substance and drug product, wherein remibrutinib drug substance and drug product comply with EMA and FDA safety or pharmaceutical product regulations (e,g. new EMA guidance on nitrosamine impurities).

In an embodiment, remibrutinib drug substance is substantially free of a genotoxic impurity, e.g., 5-fluoro-2-methyl-3-(4,4,5,5-tetramethyl-1,3,2-dioxaborolan-2-yl)aniline, and substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide.

In one aspect, the invention relates to remibrutinib drug substance being substantially free of a nitrosamine impurity, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide. In one embodiment, the invention relates to remibrutinib drug substance with a level of N-(3-(6-amino-5-(2(methyl)(nitroso)amino)ethoxy)pyrimidin-4-yl)-5-fluoro-2-methylphenyl)-4-cyclopropyl-2-fluorobenzamide being less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb.

In another aspect, the level of nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide, in remibrutinib drug substance is between about 25 ppb and about 550 ppb, e.g., between about 25 ppb and about 530 ppb; or between about 25 ppb and about 400 ppb, e.g. between about 25 ppb and about 360 ppb; or between about 25 ppb and about 300 ppb; or between about 25 ppb and about 200 ppb, e.g. between about 25 ppb and about 90 ppb; or between about 25 ppb and about 100 ppb, e.g. between about 25 ppb and about 90 ppb.

In yet another aspect, the level of nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide, in remibrutinib drug substance is between about 100 ppb and about 650 ppb; or between about 100 ppb and about 550 ppb, e.g. between about 100 ppb and about 530 ppb; or is between about 100 ppb and about 400 ppb e.g. between about 100 ppb and about 360 ppb; or is between about 100 ppb and about 350 ppb, e.g. between about 100 ppb and about 320 ppb; is between about 100 ppb and about 250 ppb; or is between about 100 ppb and about 150 ppb, e.g. between about 100 ppb and about 130 ppb.

In another aspect, the invention relates to an improved process allowing for the preparation of remibrutinib drug substance being substantially free of a nitrosamine impurity, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide, the process comprising:
a. providing a suspension comprising:

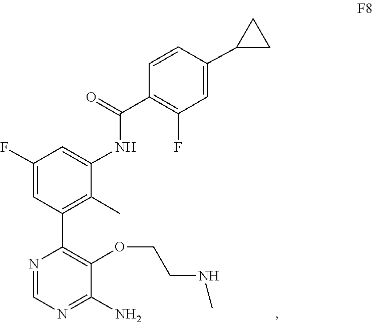 [SMILES: CNCCOc1c(N)ncnc1-c1cc(F)cc(NC(=O)c2ccc(C3CC3)cc2F)c1C]

a base, water, and a solvent;
b. reacting the suspension with acrylic anhydride to provide LOU064 drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide.

In an embodiment, the process described above is carried out while limiting the amount of nitrites in any solvents used during the preparation of the drug substance. e.g. by distilling the solvents prior to their use and/or by passing the solvents through an ion exchange resin that is capable of adsorbing nitrites.

In another embodiment, the process is carried out in the presence of about 1.1 to about 1.2 mole equivalents of base. In another embodiment, the base has a low nitrite content, e.g. less than about 250 ppb, or less than about 120 ppb. In one aspect of this embodiment, the nitrite content in the base can be determined using Griess test, e.g. as described in example 16.

In an embodiment, the process is carried out using at least about 12 mole equivalents per 1 mole of F8, e.g. at least about 25 mole equivalents, e.g. at least about 25 moles equivalent, at least about 35 moles equivalent, at least about 125 moles equivalent or at least about 150 moles equivalent per 1 mole of F8.

In an embodiment, the process is carried out in ethyl acetate as the solvent.

In further embodiments, the specifics on the reaction conditions during the steps described above are provided herein.

In an embodiment, the process further comprises steps for the preparation of F8.

In another embodiment, the invention relates to a pharmaceutical composition, comprising remibrutinib or a pharmaceutical acceptable salt thereof, and wherein the composition is substantially free of nitrosamine impurities, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide. In one aspect of this embodiment, the amount of the nitrosamine impuries (e.g. e.g. N-(3-(6-amino-5-(2(methyl)(nitroso)amino)ethoxy)pyrimidin-4-yl)-5-fluoro-2-methylphenyl)-4-cyclopropyl-2-fluorobenzamide) in the substance is less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb, all relative to the total amount of LOU064 in free form or in salt form.

In another aspect of the above embodiment, the amount of nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide, in the composition is between about 25 ppb and about 550 ppb, e.g., between about 25 ppb and about 530 ppb; or between about 25 ppb and about 400 ppb, e.g. between about 25 ppb and about 360 ppb; or between about 25 ppb and about 300 ppb; or between about 25 ppb and about 200 ppb, e.g. between about 25 ppb and about 90 ppb; or between about 25 ppb and about 100 ppb, e.g. between about 25 ppb and about 90 ppb, all relative to the total amount of LOU064 in free form or in salt form.

In yet another aspect of the above embodiment, the amount of nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide, in the composition is between about 100 ppb and about 650 ppb; or between about 100 ppb and about 550 ppb, e.g. between about 100 ppb and about 530 ppb; or is between about 100 ppb and about 400 ppb e.g. between about 100 ppb and about 360 ppb; or is between about 100 ppb and about 350 ppb, e.g. between about 100 ppb and about 320 ppb; is between about 100 ppb and about 250 ppb; or is between about 100 ppb and about 150 ppb, e.g. between about 100 ppb and about 130 ppb, all relative to the total amount of LOU064 in free form or in a salt form.

In other embodiment, the invention provides method of manufacturing the pharmaceutical composition, comprising remibrutinib, or a pharmaceutical acceptable salt thereof, (ii) one or more pharmaceutically acceptable excipients, wherein the pharmaceutical composition is substantially free of nitrosamines, particularly N-(3-(6-amino-5-(2(methyl)(nitroso)amino)ethoxy)pyrimidin-4-yl)-5-fluoro-2-methylphenyl)-4-cyclopropyl-2-fluorobenzamide. In one aspect of this embodiment, the method is carried out while limiting the amount of nitrites in solvents and excipients, e.g. nitrite levels are less than 1.5 ppm, e.g. less than 1 ppm, less than 0.5 ppm or less than 0.2 ppm.

The invention also provides pharmaceutical products comprising said compositions and documentation, e.g., in the form of packaging or a package insert. For example, a pharmaceutical product may include a document providing instructions to a patient as to how to administer the composition and/or a document which certifies that the composition is substantially free of nitrosamines, or at least substantially free of N-(3-(6-amino-5-(2(methyl)(nitroso)amino)ethoxy)pyrimidin-4-yl)-5-fluoro-2-methylphenyl)-4-cyclopropyl-2-fluorobenzamide.

These pharmaceutical compositions are suitable for the treatment of BTK related diseases as disclosed herein.

### DETAILED DESCRIPTION

Remibrutinib Drug Substance Substantially Free of Nitrosamine Impurities, Particularly Substantially Free of N-(3-(6-amino-5-(2(methyl)(nitroso)amino)ethoxy)pyrimidin-4-yl)-5-fluoro-2-methylphenyl)-4-cyclopropyl-2-fluorobenzamide

In one aspect, the invention relates to remibrutinib drug substance being substantially free of a nitrosamine impurity, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide. In one embodiment, the invention relates to remibrutinib drug substance with a level of N-(3-(6-amino-5-(2(methyl)(nitroso)amino)ethoxy)pyrimidin-4-yl)-5-fluoro-2-methylphenyl)-4-cyclopropyl-2-fluorobenzamide being less than about 1000 ppb (e.g. less than 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb), less than about 50 ppb or less than about 25 ppb.

In some embodiments, the content of nitrosamine in remibrutinib drug substance is between about 25 ppb and about 550 ppb, e.g., between about 25 ppb and about 530 ppb; or between about 25 ppb and about 400 ppb, e.g. between about 25 ppb and about 360 ppb; or between about 25 ppb and about 300 ppb; or between about 25 ppb and about 200 ppb, e.g. between about 25 ppb and about 90 ppb; or between about 25 ppb and about 100 ppb, e.g. between about 25 ppb and about 90 ppb.

In another embodiment, the content of nitrosamine in remibrutinib drug substance is between about 100 ppb and about 650 ppb; or between about 100 ppb and about 550 ppb, e.g. between about 100 ppb and about 530 ppb; or is between about 100 ppb and about 400 ppb e.g. between about 100 ppb and about 360 ppb; or is between about 100 ppb and about 350 ppb, e.g. between about 100 ppb and about 320 ppb; is between about 100 ppb and about 250 ppb; or is between about 100 ppb and about 150 ppb, e.g. between about 100 ppb and about 130 ppb.

In some further aspect of previous embodiments, remibrutinib is substantially pure (e.g. substantially chemically pure) as defined herein. In another embodiment, remibrutinib is in a crystalline form as disclosed in WO2020/234779, for example anhydrous crystalline form A as disclosed in example 1 of WO2020/234779. In one aspect of this embodiment, remibrutinib is the form of crystalline form A and is substantially phase pure. In another embodiment, remibrutinib is both substantially chemically pure and substantially phase pure. As provided herein, remibrutinib is additionally substantially free of the nitrosamine N-(3-(6-amino-5-(2(methyl)(nitroso)amino)ethoxy)pyrimidin-4-yl)-5-fluoro-2-methylphenyl)-4-cyclopropyl-2-fluorobenzamide.

The invention is also useful in the preparation of LOU064 drug substance substantially free of a nitrosamine impurity, e.g. the nitrosamine N-(3-(6-amino-5-(2(methyl)(nitroso)amino)ethoxy)pyrimidin-4-yl)-5-fluoro-2-methylphenyl)-4-cyclopropyl-2-fluorobenzamide:

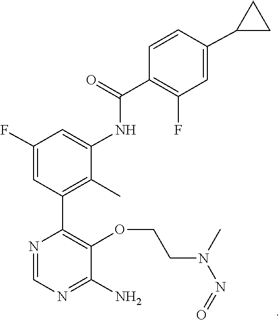 [SMILES: Cc1c(NC(=O)c2ccc(C3CC3)cc2F)cc(F)cc1-c1ncnc(N)c1OCCN(C)N=O]

In an embodiment, remibrutinib or any other compound described herein may be provided as a salt. As used herein, the terms “salt” or “salts” refers to an acid addition or base addition salt of a compound disclosed herein. “Salts” include in particular “pharmaceutically acceptable salts”.

The term “pharmaceutically acceptable salts” refers to salts that retain the biological effectiveness and properties of a compound disclosed herein and, which typically are not biologically or otherwise undesirable. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases. Inorganic bases from which salts can be derived include, for example, ammonium salts and metals from columns I to XII of the periodic table. In some embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and trimethamine. The pharmaceutically acceptable salts of the compounds disclosed herein can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

In some embodiments, remibrutinib is in a crystalline form as described in WO2020/234779, which is hereby incorporated by reference in its entirety. In an embodiment, remibrutinib is in an anhydrous crystalline form A as described in Example 1 of WO2020/234779). In another aspect of this embodiment, remibrutinib is substantially phase pure as defined herein.

Many organic solvents are suitable for the chemical reactions described herein. For example, the reactions described herein may be conducted in an aprotic organic solvent. Suitable examples include: acetonitrile; dimethylsulfoxide (DMSO); dimethylformamide (DMF); halogenated alkanes such as dichloromethane (DCM); aromatic compounds such as benzene, toluene, xylene, mesitylene, and naphthalene; alkanes such as hexane, heptane, and octane; ketones such as acetone; ether compounds such as diethyl ether, tetrahydrofuran (THF), derivatives of THF such as methyl-THF; ester compounds such as ethyl acetate and isopropylacetate; amines such as pyridine; polyethylene glycol (PEG); in particular PEG with an average molecular weight of about 100 g/mol to about 2000 g/mol such as PEG200, PEG600, PEG1000 and PEG2000, derivatives thereof such as mono- or dialkyl PEG, in particular mono- or dimethyl PEG, mono- or diethyl PEG and mono- or dipropyl PEG; and polypropylene glycol (PPG). Protic solvents may also be used in the reactions described herein. Protic solvents include: water; alcohols such as a C₁₋₁₀ aliphatic branched or linear alcohols, in particular C1-C6 alcohols; and carboxylic acids such as methanoic acid, ethanoic acid, propanoic acid, etc. In one embodiment, the solvents include toluene, ethanol, ethyl acetate isopropyl acetate, methyl-THF, heptane and isopropanol. In one embodiment, the reactions described herein are carried out avoiding non-desirable solvents such as DCM, DME, DMF, dioxane and 1,2 dichloroethane or other carcinogenic or teratogenic solvents. In certain embodiments, the amount of solvent in the reaction mixture is in the range of from 0.1% to 99% (v/v), from 0.1% to 80% (v/v), from 0.1% to 75% (v/v), from 0.1% to 50% (v/v), from 1% to 40% (v/v), from 2% to 30% (v/v), from 4% to 25% (v/v) or from 5% to 20% (v/v).

Some chemical reactions described herein can be conducted under acidic conditions, e.g. at a pH of less than 7, no more than 6, no more than 5, no more than 4, no more than 3, no more than 2, or no more than 1. Acids suitable for the chemical reactions described are known to the skilled person. Commonly used acids include inorganic acids, for example sulfuric acid, phosphoric acid, and nitric acid, boric acid; halo acids such as hydrofluoric acid, hydrochloric acid, hydrobromic acid, and hydroiodic acid; organic acids, for example carboxylic acids and derivatives thereof such as acetic acid, benzoic acid; and halogenated acetic acids such as trifluoroacetic acid, and dichloroacetic acid. In one embodiment, the acid is HF, HCl, or H₂SO₄. In another embodiment, fluorinated acids such as TFA are avoided in order to avoid generation of fluorinated waste.

Some chemical reactions described herein can be conducted under basic conditions, e.g. at a pH of greater than 7, at least 8, at least 9, at least 10, at least 11, at least 12, at least 13, or at least 14. Basic compounds suitable for the chemical reactions described herein are known to the skilled person. Commonly used bases include inorganic bases, for example hydroxides of alkali metals and alkali earth metals such as lithium hydroxide, sodium hydroxide, potassium hydroxide, magnesium hydroxide, and calcium hydroxide. Stronger bases can be made from the addition of alkali earth metals to hydrocarbons, amines, alcohols and dihydrogen. Examples include butyl lithium, lithium diisopropylamide (LDA), Lithium diethylamide (LDEA), sodium amide, sodium ethanolate, sodium hydride (NaH), and lithium bis(trimethylsilyl)amide. Weaker bases include ammonia and amines, for example trialkylamines such as triethylamine and diisopropylethylamine, and anions of weak acids such as acetates (e.g. sodium acetate), potassium acetate, and carbonates (e.g. sodium carbonate, potassium carbonate). In some embodiments, the base is inorganic and has a low nitrite content (e.g. less than about 250 ppb, less than about 120 ppb, less than about 100 ppb, e.g. about 60 ppb). Nitrite content can be determined using Griess test, e.g. as described in example 16.

The reactions described herein can be run for as long as needed to achieve completion of the reaction, or at least an acceptable yield of product. For example, the duration of the reaction may be less than 1 minute, less than 5 minutes, less than 10 minutes, less than 30 minutes, less than 1 hour, less than 2 hours, less than 3 hours, less than 5 hours, less than 10 hours, less than 20 hours, less than 30 hours less than 40 hours, less than 50 hours, or less than 60 hours. The reaction time may depend, inter alia, on the scale of the reaction. The skilled person can monitor the progress of the reaction in a number of different ways including by monitoring physical changes such as a change in color, or by monitoring the reaction using analytical methods such as NMR, FT-IR, XRPD, or chromatography, for example thin layer chromatography (TLC) or liquid chromatography coupled to mass spectrometry (LC-MS). Upon completion of the reactions described herein, the reaction mixture is optionally purified. Purification techniques are known to the skilled person and include chromatography (e.g. HPLC, which may be reverse phase or normal phase); liquid-liquid separation, for example using multiple immiscible solvents; and/or liquid-solid separation, for example using filtration, decantation, (re) crystallization, trituration, evaporation, freeze-drying.

The reactions described herein may be performed on any suitable scale. In one embodiment, the reaction mixture is of industrial scale. It may for example have a volume of at least 1 liter, in particular at least 10 liters, at least 100 liters, or at least 1000 liters. In another embodiment, the reaction mixture is on a microscale. It may for example have a volume of 10 ml or less, in particular 1 ml or less, 100 μl or less, 10 μl or less or 1 μl or less.

The reactions described herein may be part of a series of reactions comprising a synthesis. Where multiple reactions are described, these can be conducted in a sequential fashion or in a one-pot fashion. Sequential reactions typically involve the completion of a first reaction, followed by work up and purification of that reaction, before a second reaction is conducted, continuing with further reactions until the desired product has been made. In contrast, in a one-pot fashion, a first reaction may be completed, and then a second reaction may be conducted using one or more products of the first reaction without isolation. One-pot reactions are advantageous because they avoid unnecessary purification steps, saving time and materials. In the synthesis of remibrutinib drug substance described herein, some or all reactions may be conducted in a one-pot fashion, or alternatively some or all reactions may be conducted in a sequential fashion.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements may and/or indeed does encompass further elements as well as embodiments wherein the subject-matter which “comprises” specifically listed elements does not comprise further elements.

Numeric ranges described herein are inclusive of the numbers defining the range. The headings provided herein are not limitations of the various aspects or embodiments of this invention which can be read by reference to the specification as a whole. According to one embodiment, subject matter described herein as comprising certain steps in the case of methods or as comprising certain ingredients in the case of compositions refers to subject matter consisting of the respective steps or ingredients. It is preferred to select and combine specific aspects and embodiments described herein and the specific subject-matter arising from a respective combination of specific embodiments also belongs to the present disclosure.

For purposes of interpreting this specification, the following definitions will apply and whenever appropriate, terms used in the singular will also include the plural and vice versa.

The term “substantially free” of nitrosamine impurity means the content of nitrosamine in the drug product is below about 1000 ppb (parts per billion) (e.g. below about 550 ppb, below about 530 ppb, below about 400 ppb, below about 360 ppb, below about 150 ppb, below about 130 ppb, below about 100 ppb, below about 90 ppb), below about 50 ppb or below about 25 ppb. In one aspect the

The terms “ppm” and “ppb” indicates respectively part per million and part per billion. Those terms represent a weight-to-weight ratio used to describe concentration. For example, part per billion (ppb) in the drug substance is the number of units mass of a contaminant (e.g. nitrosamine impurity (e.g. nitrosamine N-(3-(6-amino-5-(2(methyl)(nitroso)amino)ethoxy)pyrimidin-4-yl)-5-fluoro-2-methylphenyl)-4-cyclopropyl-2-fluorobenzamide) per 1000 million units of total mass of drug substance. Mass of drug substance is the sum of the mass of LOU064 (free base) and the mass of all impurities (including nitrosamine impurities).

This ppb ratio can be expressed as follow:

$$\frac{Mass of nitrosamine}{Mass of drug substance} \times {10}^{9}$$

Similarly, a ppm ration can be expressed as follow:

$$\frac{Mass of nitrosamine}{Mass of drug substance} \times {10}^{6} .$$

For example, for “ppm” and “ppb” content of nitrosamine in the drug product, the content of nitrosamine is expressed relative to the amount of LOU064 in the drug product.

The amount of nitrosamine impurity (e.g. nitrosamine N-(3-(6-amino-5-(2(methyl)(nitroso)amino)ethoxy)pyrimidin-4-yl)-5-fluoro-2-methylphenyl)-4-cyclopropyl-2-fluorobenzamide) is measured using mass spectrometry (MS) coupled with separation methods (e.g. liquid chromatography or high performance liquid chromatography (HPLC)). In one embodiment, HPLC is performed using a C18 column (e.g. an octadecyl silane chemically bonded to porous or non-porous silica or ceramic micro-particles, 1.5 to 10 μm in diameter, or a monolithic rod, e.g. a USP “L1” listed column, e.g. YMC-Triart C18, 100×3.0 mm, particle size 1.9 μm, 12 nm). In one embodiment, the detection level sensitivity can be enhanced by using mass spectrometry selection ion monitoring (SIM), wherein the scanning mode is tuned to detect only selected mass of N-(3-(6-amino-5-(2(methyl)(nitroso)amino)ethoxy)pyrimidin-4-yl)-5-fluoro-2-methylphenyl)-4-cyclopropyl-2-fluorobenzamide (i.e. MH+483). In one embodiment, the determination of the nitrosamine content (i.e. N-(3-(6-amino-5-(2(methyl)(nitroso)amino)ethoxy)pyrimidin-4-yl)-5-fluoro-2-methylphenyl)-4-cyclopropyl-2-fluorobenzamide) in the drug substance is determined according to method described in example 1b or in example 15. One of skill in the art would readily understand these methods and how to employ additional (or alternative) methods for determining amounts of impurities in low ppm ranges.

The term “about”, as used herein, is intended to provide flexibility to a numerical range endpoint, providing that a given value may be “a little above” or “a little below” the endpoint accounting for variations one might see in the measurements taken among different instruments, samples, and sample preparations. The term usually means within 5%, e.g. within 1% of a given value or range. For example, 500 ppb mean 500 ppb+/−25 ppb (between 475 ppb and 525 ppb) or 500+/−5 ppb (i.e. between 495 ppb and 505 ppb)

The term “drug substance” is the substance comprising an active pharmaceutical ingredient which is intended to be used in the manufacture of a drug product (i.e. pharmaceutical composition, e.g. tablet, capsule etc.). The active pharmaceutical ingredient (i.e. LOU064 (remibrutinib)) is present in the drug substance with various degrees of purities. The terms “drug substance”, “LOU064 drug substance” and “remibrutinib drug substance” are used interchangeably. Therefore “LOU064 drug substance” is LOU064 with various degrees of purities. In one embodiment, remibrutinib is substantially pure (e.g. substantially chemically pure) as defined below. In another embodiment, remibrutinib is in a crystalline form as disclosed in WO2020/234779, for example anhydrous crystalline form A as disclosed in example 1 of WO2020/234779. In one aspect of this embodiment, remibrutinib in in the form of crystalline form A and is substantially phase pure. In another embodiment, remibrutinib is both substantially chemically pure and substantially phase pure. As provided by instant disclosure, remibrutinib is additionally substantially free of the nitrosamine N-(3-(6-amino-5-(2(methyl)(nitroso)amino)ethoxy)pyrimidin-4-yl)-5-fluoro-2-methylphenyl)-4-cyclopropyl-2-fluorobenzamide.

The terms “crystalline form(s)” or “crystalline modification(s)” or “polymorphic form(s)” or “polymorph(s)” will be used interchangeably herein. As used herein “polymorph” refers to crystalline forms having the same chemical composition but different spatial arrangements of the molecules, atoms, and/or ions forming the crystal. Each polymorph differs with respect to thermodynamic stability, physical parameters, x-ray structure and methods of preparation.

As used herein, “substantially pure,” when used in reference to LOU064, means a compound having a purity greater than 90 weight %, including greater than 90, 91, 92, 93, 94, 95, 96, 97, 98, and 99 weight %, and also including equal to about 100 weights % of remibrutinib, based on the weight drug substance. The remaining material comprises other form(s) of the compound, and/or reaction impurities and/or processing impurities arising from its preparation. For example, a crystalline form of remibrutinib may be deemed substantially pure in that it has a purity greater than 90 weight %, as measured by means that are at this time known and generally accepted in the art, where the remaining less than 10 weight % of material comprises other form(s) of remibrutinib and/or reaction impurities and/or processing impurities.

As used herein, “substantially phase pure,” when used in reference to crystalline form of LOU064, means remibrutinib having a phase purity of greater than about 90% by weight, including greater than about 90, 91, 92, 93, 94, 95, 96, 97, 98, and about 99% by weight, and also including equal to about 100% by weight of the remibrutinib, based on the weight of the LOU064 on anhydrous basis. The term “phase pure” or “phase purity” herein refers to phase homogeneity with respect to a particular solid-state form of remibrutinib and does not necessarily imply a high degree of chemical purity absent an express statement to that effect. Phase purity may be determined according to methods known in the art, for example, using XRPD to do quantitative phase analysis using one or more approaches known in the art, for example, via an external standard method, direct comparisons of line (peak) characteristics which are attributed to different phases in a particular spectra, or via an internal standard method. However, XRPD quantification of phase purity can be complicated by the presence of amorphous material. Accordingly, other methods that may be useful for determining phase purity include, for example, solid state NMR spectroscopy, Raman and/or infrared spectroscopy. One of skilled in the art would readily understand these methods and how to employ these additional (or alternative) methods for determining phase purity.

As used herein, “substantially chemically pure” when used in reference to remibrutinib, means remibrutinib having a chemical purity greater than about 90% by weight, including greater than about 90, 91, 92, 93, 94, 95, 96, 97, 98, and about 99% by weight, and also including equal to about 100% by weight of remibrutinib based on the weight of the drug substance. The remaining material generally comprises other compounds, such as for example, reaction impurities, starting materials, reagents, side products, and/or other processing impurities arising from the preparation and/or isolation and/or purification of the remibrutinib. For example, remibrutinib may be deemed to be substantially chemically pure if it has been determined to have a chemical purity of greater than about 90% by weight, as measured by standard and generally accepted methods known in the art, where the remaining less than about 10% by weight constitutes other materials such as other stereoisomers of the compound of Formula (I), reaction impurities, starting materials, reagents, side products, and/or processing impurities. Chemical purity may be determined according to methods known in the art, for example, high performance liquid chromatography (HPLC), LC-MS (liquid chromatography-mass spectrometry), nuclear magnetic resonance (NMR) spectroscopy, or infrared spectroscopy. One of skill in the art would readily understand these methods and how to employ these additional (or alternative) methods for determining chemical purity.

### Manufacturing of Remibrutinib Drug Substance Substantially Free of Nitrosamine Impurities

In one aspect, the present invention relates to a new synthetic route to remibrutinib drug substance being substantially free of N-(3-(6-amino-5-(2(methyl)(nitroso)amino)ethoxy)pyrimidin-4-yl)-5-fluoro-2-methylphenyl)-4-cyclopropyl-2-fluorobenzamide. For example, the synthetic route also avoids the formation of genotoxic intermediate INT-3. Moreover, the process minimizes purification steps, improving overall yield and providing a more efficient process. The process can also be conducted in green solvents. In one aspect, the invention provides a synthesis method for preparing LOU064 drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, comprising reacting:

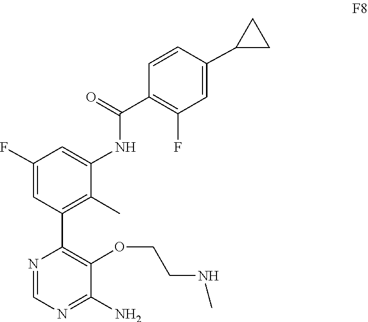 [SMILES: CNCCOc1c(N)ncnc1-c1cc(F)cc(NC(=O)c2ccc(C3CC3)cc2F)c1C]

with acrylic anhydride in a solvent and in the presence of a base to generate

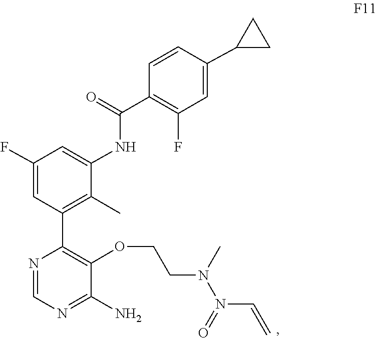

wherein water is added prior to addition of acrylic anhydride.

In one aspect, the invention comprises a process for preparing LOU064 drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, the process comprising:
c. providing a suspension comprising:

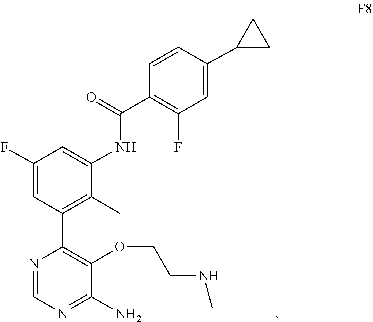 [SMILES: CNCCOc1c(N)ncnc1-c1cc(F)cc(NC(=O)c2ccc(C3CC3)cc2F)c1C]

a base, water and a solvent;
d. reacting the suspension with acrylic anhydride to provide LOU064 drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide.

The above process of converting F8 to LOU064 drug substance can be achieved in the presence of an organic base (e.g. trialkylamines such as triethylamine and diisopropylethylamine, N-methylmorpholine, N-methylpyrrolidine; aromatic heterocycles such as pyridine, N-methylimidazole; or hydroxides of quaternary ammonium cations such as tetrabutylammonium hydroxide.

The above process of converting F8 to LOU064 drug substance can be achieved in the presence of an inorganic base such as Na₂CO₃, K₂CO₃, hydroxides of alkali metals and alkali earth metals such as lithium hydroxide, sodium hydroxide, potassium hydroxide, magnesium hydroxide, and calcium hydroxide; alkali earth metals alcoholates or alkali earth metals hydride. In one aspect of this embodiment, the above reaction converting F8 to remibrutinib drug substance is achieved in the presence of Na₂CO₃.

In an embodiment, the base used in the process converting F8 to remibrutinib drug substance is a base with a low nitrite content. In some embodiment the nitrite content is less than about 250 ppb, for example less than about 120 ppb, e.g. about 60 ppb. In some embodiment, the level of nitrite in the base is determined using a Griess test, as described in example 16. For example, the base with low nitrite content is Na₂CO₃·10H₂O or K₂CO₃·7H₂O. In another embodiment, the base with low nitrite content is low nitrite content Na₂CO₃, e.g. Na₂CO₃ with a nitrite content of less than about 250 ppb, or less than about 120 ppb, e.g about 60 ppb. In another embodiment, the base with low nitrite content is low nitrite content K₂CO₃, e.g. K₂CO₃ with a nitrite content of less than about 250 ppb, or less than about 120 ppb, e.g about 60 ppb.

In an embodiment, the above process converting F8 to remibrutinib drug substance is achieved with about 1.1 mole equivalent to about 1.2 mole equivalent of base per 1 mole of intermediate F8.

The above process converting F8 to remibrutinib drug substance can be achieved in the presence of any suitable solvent. In one embodiment, the solvent can be chosen from MeTHF, THF, alcohols (isopropanol), DCM, toluene, ethylacetate, isopropylacetate, acetonitrile, acetone, tert-butylmethylether (TBME). In one aspect of this embodiment, the solvent has a low nitrite content, e.g. less than 1.5 ppm, less than 1 ppm, less than 0.5 ppm, or less than 0.25 ppm.

In an embodiment, the above process converting F8 to remibrutinib drug substance is carried out in ethyl acetate.

In an embodiment, the above process converting F8 to remibrutinib drug substance can be achieved, using sufficient amount of water to dissolve, at least partially, the base. In an embodiment, the above reaction converting F8 to remibrutinib drug substance can be achieved, e.g. using at least about 12 moles equivalent of water, or using at least about 25 moles equivalent of water prior to the addition of acrylic anhydride, or using at least about 35 moles equivalent or using at least about 125 moles equivalent, e.g. between at least about 120 moles to about 150 moles equivalent of water prior to the addition of acrylic anhydride (Step b). In an embodiment, the above process converting F8 to LOU064 drug substance is achieved with at least 150 moles equivalent of water prior to the addition of acrylic anhydride (step b).

In an embodiment, the water used in the process (Step a) is purified water (e.g. distilled water), e.g. purified water at a level of nitrite of less than about 50 ppb, e.g. less than 20 ppb.

In some embodiments, the above process converting F8 to remibrutinib drug substance can be carried out at room temperature or can be heated at temperature up to just below the boiling point of chosen solvent. For example, when ethyl acetate is used as a solvent, the reaction mixture can be heated at a temperature of about 50° C. to about 65° C.

In some embodiments, F8 and the base are suspended in the solvent and water is added to form a suspension. The suspension can be kept at room temperature or can be heated prior to addition of acrylic anhydride. For example, F8 and Na₂CO₃ are suspended in ethyl acetate and added water and heated to a temperature of about 50 to about 65° C. prior to the addition of acrylic anhydride. In an embodiment, acrylic anhydride is added in solution in a suitable solvent, for example in same solvent used to dissolve or suspend F8 and the base. In an embodiment, acrylic anhydride is added slowly to the solution or suspension of F8, base and water in solvent.

In an embodiment, acrylic anhydride is dissolved in ethyl acetate and the solution of acrylic anhydride in ethyl acetate is added slowly to the suspension of F8, Na₂CO₃ in ethyl acetate and water.

In one embodiment, the drug substance obtained by the above process is substantially free of a nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide. In another aspect of this embodiment, the content of nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide in the drug substance obtained by above process is less than about 550 ppb, e.g. less than about 530 ppb; less than about 400 ppb, e.g. less than about 360 ppb; less than about 300 ppb; less than about 200 ppb; less than about 150 ppb, e.g. less than about 130 ppb. In another aspect of this embodiment, the content of nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide in the drug substance obtained by above process is between about 100 ppb and about 650 ppb; or between about 100 ppb and about 550 ppb, e.g. between about 100 ppb and about 530 ppb; or is between about 100 ppb and about 400 ppb e.g. between about 100 ppb and about 360 ppb; or is between about 100 ppb and about 350 ppb, e.g. between about 100 ppb and about 320 ppb; is between about 100 ppb and about 250 ppb; or is between about 100 ppb and about 150 ppb, e.g. between about 100 ppb and about 130 ppb.

In one aspect, the instant invention further comprises steps of forming the intermediate F8.

### Preparation of F8 by Deprotection of F7:

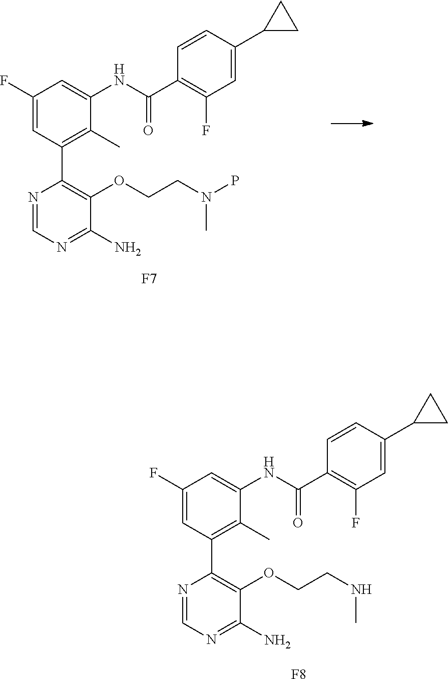

wherein P is an amine protecting group.

In one aspect, F7 is deprotected using acidic conditions (e.g. in the presence of HCl). F8 can then be isolated after a neutralization step.

It has been discovered that the nitrosamine content of Intermediate F8 increases upon exposure to the air and/or upon storage (Example 5 on stability studies of F8)

In one aspect of the invention, the nitrosamine content in F8 is decreased by utilizing purified water (e.g. distilled water) and pure NaOH base (e.g. low nitrite content NaOH, e.g. less than about 250 ppb, less about than 120 ppb (e.g. by using a fresh bottle of NaOH, e.g. as determined using a Griess test—example 16) during the neutralization step (example 5). In one aspect, the content of nitrosamine in F8 is reduced to e.g. less than about 100 ppb, less than about 80 ppb, less than about 50 ppb, less than about 40 ppb, e.g. about 30 ppb.

In another aspect of the invention, the intermediate F8 is isolated but is used directly without drying for the conversion step F8->drug substance as described above. (i.e. F8 is added into the conversion step F8 to drug substance (step a) as described above without any drying step—Example 6). Drying F8 and further exposition to the air, can increase the content of nitrosamine. In one aspect of this embodiment, the content of nitrosamine in the drug substance is reduced to e.g. less than about 100 ppb, less than about 50 ppb, less than about 30 ppb, e.g. about to 25 ppb.

The instant invention further comprises steps of forming the intermediate F7. Methods of making F7 have been described in PCT/IB2023/059664, which is herein incorporated by reference in its entirety.

### Preparation of F7 by Reacting Compound X6b and Compound F6:

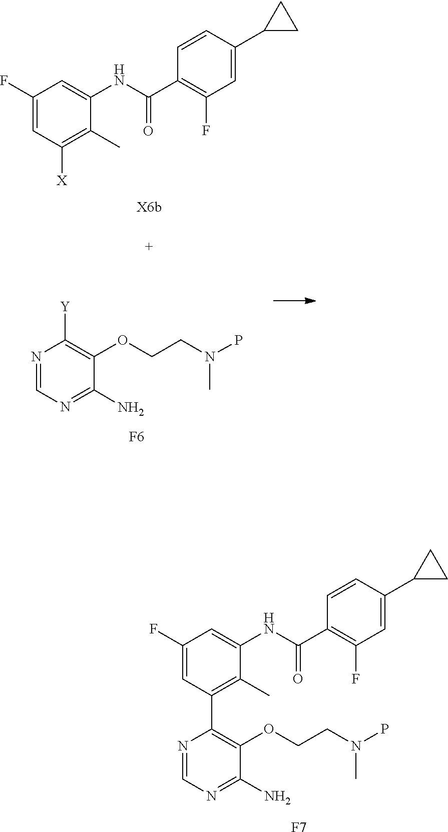

wherein X and Y are each independently Cl, Br, or I, (e.g. Br) and wherein P is an amine protecting group.

In some embodiments, X is Cl or Br. In some embodiments, Y is Cl or Br. In some embodiments, X and Y are each Cl or Br. In some embodiments, X is Br. In some embodiments, Y is Cl. In some embodiments, X is Br and Y is Cl. These embodiments apply to any and all instances of X and Y described herein, including X and Y groups present on synthetic precursors to X6b and F6, respectively.

The protecting group P can be any suitable amine protecting group that is stable during any of the chemical transformations described herein (except for deprotection steps). Amine protecting groups may be removed by conditions, for example acid, base, hydrogenation, light, heat, etc. Examples of suitable amine protecting groups include carbamate protecting groups, such as 9-fluorenylmethyl carbamate (Fmoc), t-butyl carbamate (Boc), or benzyl carbamate (Cbz); acetamide protecting groups such as acetamide, trifluoroacetamide, or benzylamide; and sulfonamide protecting groups such as p-toluenesulfonamide.

X6b and F6 can be converted to F7 according to coupling conditions suitable for forming a carbon-carbon bond. For example, the coupling of X6b and F6 can be achieved using an organometallic cross-coupling reaction whereby the two fragments are joined together with the aid of a metal catalyst. Cross-coupling conditions that could be employed in the coupling of X6b and F6 include Kumada coupling; Negishi coupling; Stille coupling; Suzuki-Miyaura coupling, and Hiyama coupling. In a typical cross-coupling reaction, a compound of the type R-M (R=first organic fragment, M=metal or main group compound) reacts with an organic halide of the type R′—X (R′=second organic fragment, X=halide) with formation of a new carbon-carbon bond in the product R—R′.

Thus, in some embodiments, the preparation of F7 comprises the conversion of F6 into pre-cursor F6′ by replacement of Y with “M”, a metal-containing moiety, or a main group element containing moiety, for example wherein M contains Zn (Negishi) B (Suzuki-Miyaura), Mg (Kumada), Sn (Stille), or Si (Hiyama):

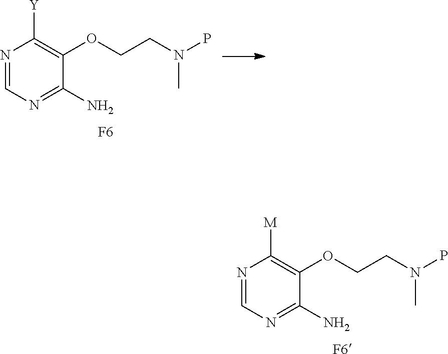

wherein P is an amine protecting group, e.g. Boc.

F6′ can be reacted with X6b under cross-coupling conditions to give F7. In some embodiments, the conversion of F6 to F6′ and the cross-coupling of F6′ with X6b is conducted in a one-pot reaction. In some embodiments, the conversion of F6 to F6′ and the cross-coupling of F6′ with X6b are conducted in sequential reactions.

Alternatively, the preparation of F7 comprises the conversion of X6b into a precursor compound X6b′ by replacement of X with “M”, a metal-containing moiety, or a main group element-containing moiety, for example wherein M contains Zn (Negishi) B (Suzuki-Miyaura), Mg (Kumada), Sn (Stille), or Si (Hiyama):

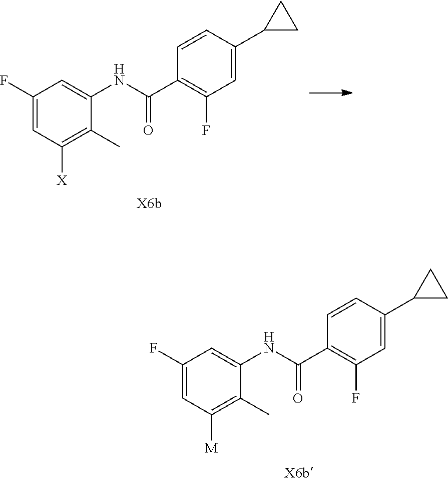

The precursor compound X6b′ can be reacted with F6 under cross-coupling conditions to give F7. In some embodiments, the conversion of X6b to X6b′ and the cross-coupling of X6b′ with F6 is conducted in a one-pot reaction. In some embodiments, the conversion of X6b to X6b′ and the cross-coupling of X6b′ with F6 are conducted in sequential reactions.

Preparation of X6a—borylation Reaction

The invention further provides a synthesis method comprising the borylation of X6b to give X6a:

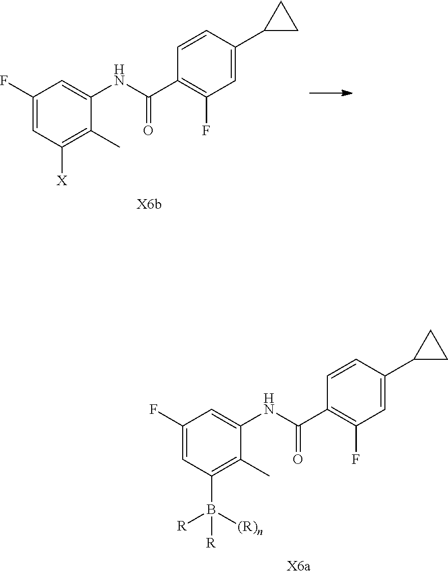

wherein X is F, Cl, Br, or I; n is 0 or 1 and R is F, Cl, Br, or I, OH, OC₁-C₆ alkyl, N(C₁-C₆ alkyl)₂, aryl, or wherein two or three R groups other than F, Cl, Br, I, or OH can be taken together to form a cyclic boronate ester, for example pinacol boronate, or N-methyliminodiacetic acid (MIDA) boronate.

The borylation of X6b can be achieved using one or more catalysts, one or more ligands, one or more borylating agents, one or more bases, and/or one or more additives. In some embodiments, the borylation includes one or more catalysts, one or more ligands, one or more borylating agents, and one or more bases. In some embodiments, the borylation additionally includes one or more additives.

Borylating agents are boron-containing compounds that are capable of converting organohalide compounds into boronic acids or boronic esters, usually under metal-catalysed cross-coupling conditions. In some embodiments, the borylating agent is selected from the group consisting of diboron compounds, boronic acids, boranes, boron trihalides, and borates. In some embodiments, the borylating agent is selected from the group consisting of bis(pinacolato)diboron, B₂(NMe₂)₄, B₂F₄, B₂Cl₄, B₂Br₄, B₂I₄, bis-boronic acid, pinacolborane, HB(NMe₂)₂, B(OH)₃, BF₃, BCl₃, BBr₃, Bl₃, mono-, di-, or tri-C₁-C₆alkylborate, mono-, di-, or tri-methylborate, mono-, di-, or tri-ethylborate, and mono-, di-, or tri-propylborate, e.g. bis(pinacolato)diboron or bis-boronic acid. The use of bis-boronic acid may be attractive as it can allow for lower catalyst loadings, milder reaction conditions, and avoids the formation of pinacol-related impurities, as compared to pinacolborane or bis(pinacolato)diboron. It also allows for the use of green solvent such as alcoholic solvent and milder reaction condition (e.g. lower temperature).

The metal catalyst used in the borylation reaction may contain palladium, nickel, or copper, or a combination thereof, e.g. palladium.

In some embodiments, the metal catalyst is provided as a pre-catalyst complex, for example a Buchwald G1, G2, G3, or G4 pre-catalyst complexed to a phosphine ligand. Buchwald pre-catalysts are used to generate active Pd(0) in situ via rapid deprotonation and reductive elimination. Pre-catalysts are useful as they allow low catalytic loadings and are stable to air, moisture and heat with good solubility. These pre-catalysts have been optimised to further enhance function and solubility from Generations 1 to 4 (G1 to G4). The pre-catalysts comprise of a palladacycle (shown below) with a phenyl or 1,1-biphenyl backbone, wherein L represents a bound phosphine ligand eg. XPhos, SPhos, etc. (see below) and wherein the bound amine substituents and the leaving group (Cl, OMs) vary based on the Generation.

Examples of Buchwald pre-catalysts complexed with palladium and with an exemplary XPhos ligand are shown below:

| | Pre-catalyst | | Complex with XPhos | |
|---|---|---|---|---|
| G# | Formula | Structural name | Formula | Structural name |
| G1 | 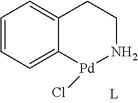 | chloropalladium(1+); (L);2-phenylethan- amine | 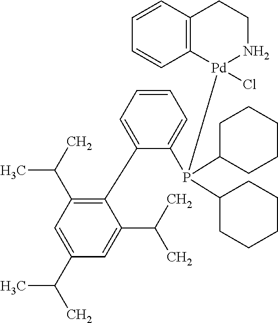 | chloropalladium(1+); dicyclohexyl-[2-[2,4,6- tri(propan-2-yl)phenyl] phenyl]phosphane; 2- phenylethanamine |
| G2 | 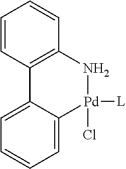 | chloropalladium(1+); (L);2-phenylaniline | 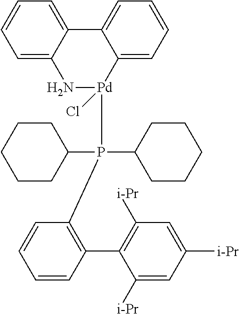 | chloropalladium(1+); dicyclohexyl-[2-[2,4,6- tri(propan-2-yl)phenyl] phenyl]phosphane; 2- phenylaniline |
| G3 | 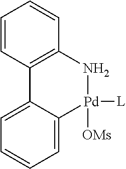 | (L);methanesulfonate; palladium(2+);2- phenylaniline | 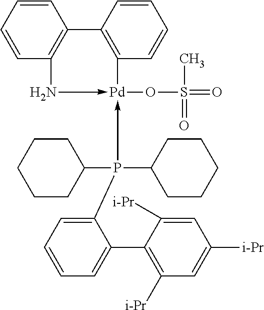 | dicyclohexyl-[2-[2,4,6- tri(propan-2-yl)phenyl] phenyl]phosphane; methanesulfonate; palladium(2+);2-phen- ylaniline |
| G4 | 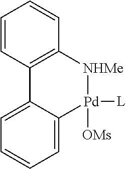 | (L);methanesulfonate; N-methyl-2-phenyl- aniline; palladium(2+) | 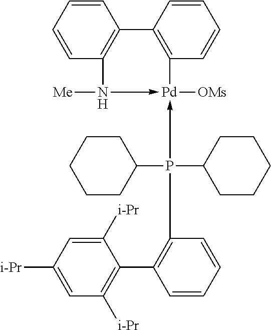 | dicyclohexyl-[2-[2,4,6- tri(propan-2-yl)phenyl] phenyl]phosphane; methanesulfonate;N- methyl-2-phenyl- aniline;palladium(2+) |

Any other phosphine ligand described herein may be used as L instead of XPhos in the table above.

Other precatalysts for borylation may include Pd(TFA)₂, PdBr₂ or Pd(MeCN)₂Cl₂. This precatalysts can be used in the presence of ligands such as Ph₂P(t-Bu); Cy₃P-HBF₄; RuPHOS; S-PHOS, Cy-BIPHEP; SPHOS-SO₃Na.

In some embodiments, the borylation of X6b involves a further ligand in addition to the ligand L forming part of the pre-catalyst complex. In other embodiments, no further ligand is required. In some embodiments, the borylation of X6b uses a catalyst and a ligand without a pre-catalyst (Pd(0) catalyst; e.g. Pd(PPh₃)₄).

A wide range of ligands can be used in borylation reactions, and the ligand can affect the reactivity of the reagents. For example, ligands can increase the electron density at the metal center of the metal complex, which can improve the oxidative addition step. In addition, a bulky ligand helps in the reductive elimination step. In some embodiments, the ligand used in the borylation of X6b is selected from the group consisting of organophosphines, N-heterocyclic carbenes, diazabutadiene, dibenzylideneacetone, and combinations thereof.

In a one embodiment, the ligand is an organophosphine ligand, for example an organophosphine selected from the group consisting of XPhos, APhos, CPhos, RuPhos, SPhos, cataCXium, DavePhos, JohnPhos, MePhos, XantPhos, Cy₃P-HBF₄, Cy-BIPHEP, SPhos-SO₃Na, PPh₃, tBuPPh₂ and combinations thereof, e.g. XPhos, APhos, CPhos, RuPhos, SPhos, cataCXium, e.g. XPhos, cataCXium and tBuPPh₂, e.g. tBuPPh₂. Phosphine ligands are depicted in the table below:

| Phosphine | Formula | IUPAC Name |
|---|---|---|
| XPhos | 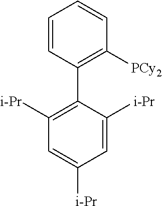 | dicyclohexyl-[2- [2,4,6-tri(propan- 2-yl)phenyl] phenyl]phosphane |
| RuPhos | 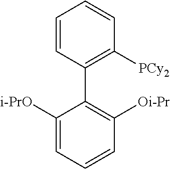 | dicyclohexyl-[2- [2,6-di(propan-2- yloxy)phenyl] phenyl]phosphane |
| SPhos | 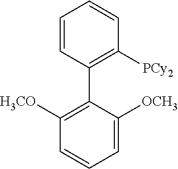 | dicyclohexyl-[2- (2,6-dimethoxy- phenyl)phenyl] phosphane |
| DavePhos | 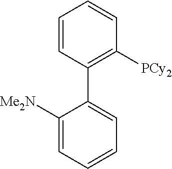 | 2-(2-dicyclohex- ylphosphanyl- phenyl)-N,N- dimethylaniline |
| JohnPhos | 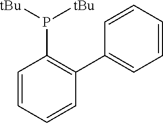 [SMILES: CC(C)(C)P(c1ccccc1-c1ccccc1)C(C)(C)C] | ditert-butyl-(2- phenylphenyl)- phosphane |
| MePhos | 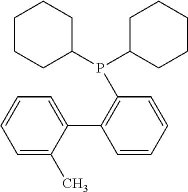 [SMILES: Cc1ccccc1-c1ccccc1P(C1CCCCC1)C1CCCCC1] | dicyclohexyl-[2- (2-methylphen- yl)phenyl]phos- phane |
| CataCXium | 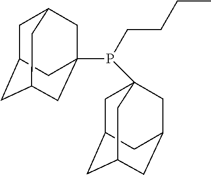 [SMILES: CCCCP(C12CC3CC(CC(C3)C1)C2)C12CC3CC(CC(C3)C1)C2] | bis(1-adamantyl)- butylphosphane |
| APhos | 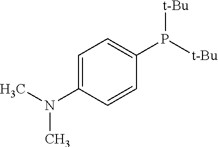 [SMILES: CN(C)c1ccc(P(C(C)(C)C)C(C)(C)C)cc1] | 4-ditert-butyl- phosphanyl-N,N- dimethylaniline |
| XantPhos | 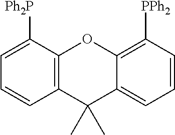 | (5-diphenylphos- phanyl-9,9- dimethylxanthen- 4-yl)-diphenyl- phosphane |
| PPh₃ | 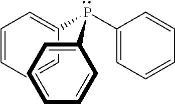 | triphenylphos- phine |
| t-BuPPh2 | 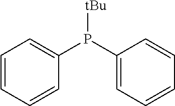 [SMILES: CC(C)(C)P(c1ccccc1)c1ccccc1] | Tertbutyldiphenyl- phosphine |
| PCy3•HBF4 | 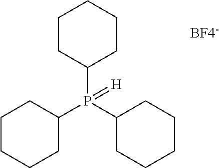 | Tricyclohexyl- phosphine tetra- fluoroborate |
| Cy-BIPHEP | 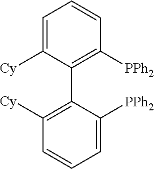 | (6,6′-Dicyclohex- ylbiphenyl-2,2′- diyl)-bis-(diphen- ylphosphine) |

The borylation of X6b can involve a base. In some embodiments, the base is an organic or inorganic salt such as NaOH, Ca(OH)₂, Na₂CO₃, K₂CO₃, K₃PO₄, Cs₂CO₃, KOAc, KOPh, or NaOAc, a tertiary amine, such as diisopropylethylamine (DIPEA), triethylamine, or a combination thereof. In one embodiment, the base is DIPEA, KOAc or KOH, e.g. KOAc.

The borylation of X6b can involve an additive, for example an alcohol such as ethylene glycol. In some embodiments, the borylation of X6b does not involve an additive.

The borylation of X6b can be conducted in any suitable solvent. Examples of suitable organic solvents include polar solvents, non-polar solvents, protic solvents, aprotic solvents, polar protic solvents, and polar aprotic solvents. In one embodiment, borylation can be conducted in alcoholic solvents, including t-amyl alcohol, hexanol, pentanol, butanol (tert-butanol, isobutanol, and n-butanol), propanol (isopropanol and n-propanol), ethanol, and/or methanol. In one embodiment, the borylation is conducted in methanol, toluene, and/or MeTHF, e.g. in MeTHF. Other solvents can also be used, for example halogenated alkane solvents such as dichloromethane. Ether-based solvents such as dioxane, MeTHF, THF, and dialkylethers such as diethylether, can also be used. Borylation can also be conducted in an aqueous environment, including a micellar environment. In some embodiments, a mixture of solvents is used.

The borylation of X6b can be achieved using one or more catalysts, one or more ligands, one or more borylating agents, one or more bases, and/or optionally one or more additives. The skilled person can determine appropriate amounts of these reagents. Nonetheless, in some embodiments of the borylation reaction:
i) The catalyst or pre-catalyst is present in an amount of 0.01 mol % to 3 mol %, 0.05 mol % to 2 mol %, 0.1 mol % to 2 mol %, 0.1 to 1 mol %, preferably 0.25 mol % and more preferably 0.5 mol % relative to the number of moles of X6b;
ii) The ligand is in an amount of 0.02 mol % to 6 mol %, 0.1 mol % to 2 mol %, 0.2 mol % to 1 mol %, 0.5 mol % or 1 mol % relative to the number of moles of X6b;
iii) The number of moles of ligand is twice or 3 times the number of moles of catalyst or pre-catalyst; preferably twice;
iv) The borylating agent is in an amount of 1 to 3 molar equivalents compared to X6b, preferably in an amount of 1 to 2 molar equivalent, more preferably 1.05 or 1.5 molar equivalents compared to X6b;
v) The base is in an amount of 2 to 5 molar equivalents, preferably 2 to 3 molar equivalent, most preferably 2.5 or 3 molar equivalents relative to the number of moles of X6b; and/or
vi) The additive is optional and when present is in an amount of 2 to 5 molar equivalents compared to X6b; preferably the additive is absent.

The borylation reaction may be characterized by any one of i) to vi) above. The borylation reaction may be characterized by any two of i) to vi) above. The borylation reaction may be characterized by any three of i) to vi) above. The borylation reaction may be characterized by any four of i) to vi) above. The borylation reaction may be characterized by any five of i) to vi) above. The borylation reaction may be characterized by all of i) to vi) above.

The borylation reaction may be characterized by i) and ii) above. The borylation reaction may be characterized by i) and iii) above. The borylation reaction may be characterized by i) and iv) above. The borylation reaction may be characterized by i) and v) above. The borylation reaction may be characterized by i) and vi) above. The borylation reaction may be characterized by ii) and iii) above. The borylation reaction may be characterized by ii) and iv) above. The borylation reaction may be characterized by ii) and v) above. The borylation reaction may be characterized by ii) and vi) above. The borylation reaction may be characterized by iii) and iv) above. The borylation reaction may be characterized by iii) and v) above. The borylation reaction may be characterized by iii) and vi) above. The borylation reaction may be characterized by iv) and v) above. The borylation reaction may be characterized by iv) and vi) above. The borylation reaction may be characterized by v) and vi) above.

In one example, a borylation reaction having excellent yield and minimal by-products may be:

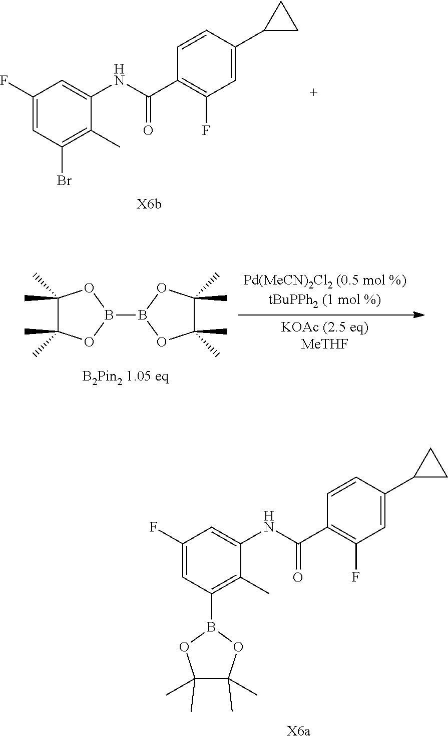

In one embodiment, a borylation reaction having excellent yield and minimal-by product formation is characterized by at least one of the following:
i) the catalyst is Pd(MeCN)₂Cl₂ in an amount of 0.1 mol % to 2 mol % relative to the number of moles of X6b, or 0.1 mol % to 1.5 mol %, preferably 0.25 mol % or more preferably 0.5 mol % relative to the number of moles of X6b;
ii) the ligand is tBuPPh₂ in an amount of 0.2 mol % to 4% relative to the number of moles of X6b, 0.2 mol % to 3 mol %, preferably 0.5 mol % or more preferably 1 mol % relative to the number of moles of X6b;
iii) the catalyst is Pd(MeCN)₂Cl₂, the ligand is tBuPPh₂ and the number of moles of tBuPPh₂ is twice or three times the number of moles of Pd(MeCN)₂Cl₂, preferably twice the number of moles of Pd(MeCN)₂Cl₂;
iv) the borylating agent is bis(pinacolato)diboron in an amount of 1 to 2 molar equivalents relative to X6b, preferably about 1.05 molar equivalents relative to X6b;
v) the base is KOAc in an amount of 2 to 5 molar equivalents relative to X6b, preferably 2.5 equivalents relative to X6b; and
vi) no additive is present; and/or
vii) the temperature of the reaction is 30° C. to 120° C., e.g. 40° C. to 50° C., preferably 60° C. or 70° C.

The borylation reaction may be characterized by any one of i) to vii) above. The borylation reaction may be characterized by any two of i) to vi) above. The borylation reaction may be characterized by any three of i) to vii) above. The borylation reaction may be characterized by any four of i) to vii) above. The borylation reaction may be characterized by any five of i) to vii) above. The borylation reaction may be characterized by any six of i) to vii) above. The borylation reaction may be characterized by all of i) to vii) above.

The borylation reaction may be characterized by i) and ii) above. The borylation reaction may be characterized by i) and iii) above. The borylation reaction may be characterized by i) and iv) above. The borylation reaction may be characterized by i) and v) above. The borylation reaction may be characterized by i) and vi) above. The borylation reaction may be characterized by i) and vii) above. The borylation reaction may be characterized by ii) and iii) above. The borylation reaction may be characterized by ii) and iv) above. The borylation reaction may be characterized by ii) and v) above. The borylation reaction may be characterized by ii) and vi) above. The borylation reaction may be characterized by ii) and vii) above. The borylation reaction may be characterized by iii) and iv) above. The borylation reaction may be characterized by iii) and v) above. The borylation reaction may be characterized by iii) and vi) above. The borylation reaction may be characterized by iii) and vii) above. The borylation reaction may be characterized by iv) and v) above. The borylation reaction may be characterized by iv) and vi) above. The borylation reaction may be characterized by iv) and vii) above. The borylation reaction may be characterized by v) and vi) above. The borylation reaction may be characterized by v) and vii) above. The borylation reaction may be characterized by vi) and vi) above.

In one embodiment, a borylation reaction having a good yield and minimal by-product formation is characterized by at least one of the following:
i) the catalyst is a pre-catalyst which is Pd-XPhos-2G in an amount of is 0.05 mol % to 0.5 mol % relative to the number of moles of X6b, preferably 0.25 mol % relative to the number of moles of X6b;
ii) the ligand is XPhos in an amount of 0.1 mol % to 1 mol % relative to the number of moles of X6b; preferably 0.5 mol % relative to the number of moles of X6b;
iii) the catalyst is Pd-XPhos-2G, the ligand is XPhos, and the number of moles of XPhos is twice the number of moles of Pd-XPhos-2G;
iv) the borylating agent is bis-boronic acid in an amount of 1 to 3 molar equivalents compared to X6b, preferably 1.5 molar equivalents compared to X6b;
v) the base is potassium acetate in an amount of 2 to 5 molar equivalents, preferably 3 molar equivalents relative to X6b;
vi) the additive is ethylene glycol in an amount of 2 to 5 molar equivalents compared to X6b, preferably 3 molar equivalents relative to X6b; and
vii) the temperature of the reaction is 30° C. to 70° C., preferably 40° C. to 50° C., more preferably 50° C.

The borylation reaction may be characterized by any one of i) to vii) above. The borylation reaction may be characterized by any two of i) to vi) above. The borylation reaction may be characterized by any three of i) to vii) above. The borylation reaction may be characterized by any four of i) to vii) above. The borylation reaction may be characterized by any five of i) to vii) above. The borylation reaction may be characterized by any six of i) to vii) above. The borylation reaction may be characterized by all of i) to vii) above.

The borylation reaction may be characterized by i) and ii) above. The borylation reaction may be characterized by i) and iii) above. The borylation reaction may be characterized by i) and iv) above. The borylation reaction may be characterized by i) and v) above. The borylation reaction may be characterized by i) and vi) above. The borylation reaction may be characterized by i) and vii) above. The borylation reaction may be characterized by ii) and iii) above. The borylation reaction may be characterized by ii) and iv) above. The borylation reaction may be characterized by ii) and v) above. The borylation reaction may be characterized by ii) and vi) above. The borylation reaction may be characterized by ii) and vii) above. The borylation reaction may be characterized by iii) and iv) above. The borylation reaction may be characterized by iii) and v) above. The borylation reaction may be characterized by iii) and vi) above. The borylation reaction may be characterized by iii) and vii) above. The borylation reaction may be characterized by iv) and v) above. The borylation reaction may be characterized by iv) and vi) above. The borylation reaction may be characterized by iv) and vii) above. The borylation reaction may be characterized by v) and vi) above. The borylation reaction may be characterized by v) and vii) above. The borylation reaction may be characterized by vi) and vii) above.

In another example, the borylation reaction may be:

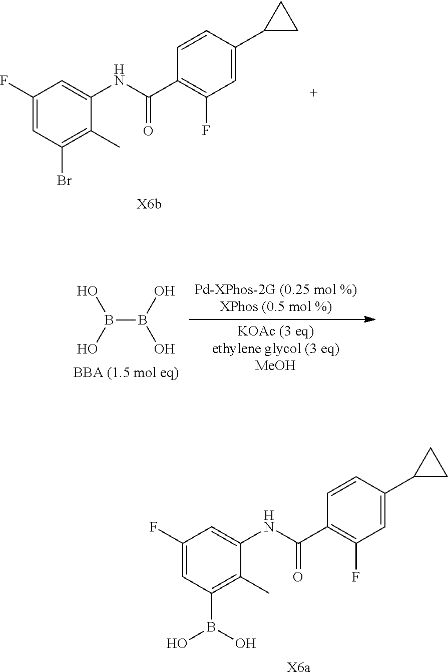 [SMILES: Cc1c(Br)cc(F)cc1NC(=O)c1ccc(C2CC2)cc1F ; "X6b" ; OB(O)B(O)O ; Cc1c(NC(=O)c2ccc(C3CC3)cc2F)cc(F)cc1B(O)O ; "X6a"]

In one embodiment, a borylation reaction having good yield and minimal by-product formation is characterized by at least one of the following:
i) the catalyst is Pd-cataCXium-3G in an amount of 0.001 mol % to 0.5 mol % relative to the number of moles of X6b, preferably 0.05 mol % relative to the number of moles of X6b;
ii) the ligand is cataCXium in an amount of 0.02 mol % to 1% relative to the number of moles of X6b, preferably 0.1 mol % relative to the number of moles of X6b;
iii) the catalyst is Pd-cataCXium-3G, the ligand is cataCXium and the number of moles of cataCXium is twice the number of moles of Pd-cataCXium-3-3G;
iv) the borylating agent is bis-boronic acid in an amount of 1 to 3 molar equivalents relative to X6b, preferably 1.5 molar equivalents relative to X6b;
v) the base is N,N-diisopropylethylamine in an amount of 2 to 5 molar equivalents relative to X6b, preferably equivalents relative to X6b; and
vi) no additive is present; and/or
vii) the temperature of the reaction is 30° C. to 70° C., preferably 40° C. to 50° C., more preferably 50° C.

The borylation reaction may be characterized by any one of i) to vii) above. The borylation reaction may be characterized by any two of i) to vi) above. The borylation reaction may be characterized by any three of i) to vii) above. The borylation reaction may be characterized by any four of i) to vii) above. The borylation reaction may be characterized by any five of i) to vii) above. The borylation reaction may be characterized by any six of i) to vii) above. The borylation reaction may be characterized by all of i) to vii) above.

The borylation reaction may be characterized by i) and ii) above. The borylation reaction may be characterized by i) and iii) above. The borylation reaction may be characterized by i) and iv) above. The borylation reaction may be characterized by i) and v) above. The borylation reaction may be characterized by i) and vi) above. The borylation reaction may be characterized by i) and vii) above. The borylation reaction may be characterized by ii) and iii) above. The borylation reaction may be characterized by ii) and iv) above. The borylation reaction may be characterized by ii) and v) above. The borylation reaction may be characterized by ii) and vi) above. The borylation reaction may be characterized by ii) and vii) above. The borylation reaction may be characterized by iii) and iv) above. The borylation reaction may be characterized by iii) and v) above. The borylation reaction may be characterized by iii) and vi) above. The borylation reaction may be characterized by iii) and vii) above. The borylation reaction may be characterized by iv) and v) above. The borylation reaction may be characterized by iv) and vi) above. The borylation reaction may be characterized by iv) and vii) above. The borylation reaction may be characterized by v) and vi) above. The borylation reaction may be characterized by v) and vii) above. The borylation reaction may be characterized by vi) and vii) above.

For example, the reaction may be:

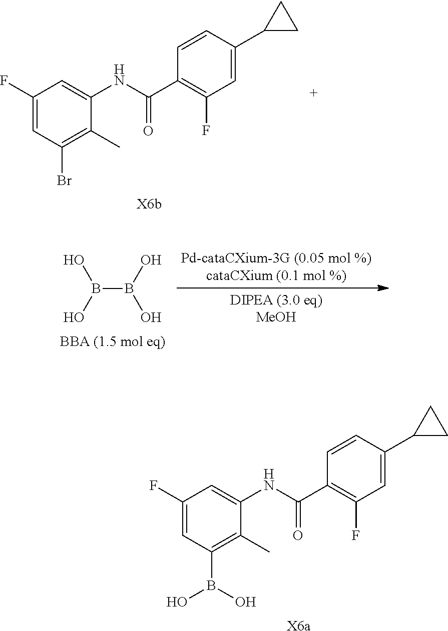 [SMILES: Cc1c(Br)cc(F)cc1NC(=O)c1ccc(C2CC2)cc1F ; "X6b" ; OB(O)B(O)O ; Cc1c(NC(=O)c2ccc(C3CC3)cc2F)cc(F)cc1B(O)O ; "X6a"]

### Coupling of X6a and F6

In some embodiments of the invention, the borylation of X6b to give X6a is used in a method of synthesising compound F7. In such embodiments, X6b is converted to X6a, and then X6a is reacted with F6 under cross-coupling conditions to generate F7. In one embodiment, the conversion of X6b to X6a and the cross-coupling of X6a with F6 is conducted in a one-pot reaction. In some embodiments, the conversion of X6b to X6a and the cross-coupling of X6a with F6 are conducted in sequential reactions.

According to the invention, borylated compound X6a can be reacted with aryl halide in a cross-coupling reaction. In one embodiment, the coupling reaction is conducted using one or more catalysts, one or more ligands, one or more bases, and/or one or more additives. In one embodiment, the coupling reaction is conducted using one or more catalysts, one or more ligands, and one or more bases. In some embodiments, the coupling additionally includes one or more additives.

The metal catalyst used in the cross-coupling reaction may contain palladium, nickel, or copper, or a combination thereof, e.g. palladium.

A wide range of ligands can be used in the cross-coupling of X6a and F6, and the ligand can affect the reactivity of the coupling reagents. For example, ligands can increase the electron density at the metal center of the metal complex, which can improve the oxidative addition step. In addition, a bulky ligand helps in the reductive elimination step. In some embodiments, the ligand used in the coupling of X6a and F6 is selected from the group consisting of organophosphines, N-heterocyclic carbenes, diazabutadiene, dibenzylideneacetone, and combinations thereof. In one embodiment, the ligand is an organophosphine ligand, for example an organophosphine selected from the group consisting of XPhos, APhos, CPhos, RuPhos, SPhos, cataCXium, DavePhos, JohnPhos, MePhos, XantPhos, PPh₃, tBuPPh₂ and combinations thereof. In another embodiment, the ligand is XPhos, APhos, CPhos, RuPhos, SPhos, cataCXium, e.g. XPhos, cataCXium and tBuPPh₂. In yet another embodiment, the ligand is tBuPPh₂.

In the coupling of X6a and F6, the metal catalyst and ligand may be provided as a pre-catalyst complex, for example a Buchwald G1, G2, G3, or G4 pre-catalyst, preferably G2, complexed to a phosphine ligand, for example an organophosphine selected from the group consisting of XPhos, APhos, CPhos, RuPhos, SPhos, cataCXium, DavePhos, JohnPhos, MePhos, XantPhos, Cy₃P-HBF₄, Cy-BIPHEP, SPHOS-SO₃Na, PPh₃, tBuPPh₂ and combinations thereof. In one embodiment, the organophosphine is XPhos, APhos, CPhos, RuPhos, SPhos, cataCXium, e.g. XPhos, cataCXium and tBuPPh₂. In another embodiment, the organophosphine is tBuPPh₂.

In some embodiments, a pre-catalyst containing a phosphine ligand is used and no additional phosphine ligand is used. Alternatively, a pre-catalyst containing a phosphine ligand is used and additional phosphine ligand is also used. Examples of pre-catalysts for cross-coupling reaction may include Pd(TFA)₂, PdBr₂ or Pd(MeCN)₂Cl₂. These pre-catalysts can be used in the presence of ligands such as Ph₂P (t-Bu); Cy₃P-HBF₄; RuPHOS; S-PHOS, Cy-BIPHEP; SPHOS-SO₃Na.

The coupling of X6a and F6 can involve a base. In some embodiments, the base is an organic or inorganic salt such as KOH, NaOH, Ca(OH)₂, Na₂CO₃, K₂CO₃, K₃PO₄, Cs₂CO₃, KOAc, KOPh, or NaOAc, a tertiary amine, such as diisopropylethylamine (DIPEA), triethylamine, or a combination thereof. In one embodiment, the base is triethylamine or KOH, e.g. KOH.

The coupling of X6a and F6 can optionally involve an additive, for example an alcohol such as ethylene glycol, for example when PdXPhos-2G/XPhos complex is used.

The coupling of X6a and F6 can be conducted in any suitable solvent. Examples of suitable organic solvents include polar solvents, non-polar solvents, protic solvents, aprotic solvents, polar protic solvents, and polar aprotic solvents. In one embodiment, cross-coupling reaction can be conducted in alcoholic solvents, including t-amyl alcohol, hexanol, pentanol, butanol (tert-butanol, isobutanol, and n-butanol), propanol (isopropanol and n-propanol), ethanol, and/or methanol. Other solvents can also be used, for example halogenated alkane solvents such as dichloromethane. Ether-based solvents such as dioxane, MeTHF, THF, and dialkylethers such as diethylether, can also be used. The coupling can also be conducted in an aqueous environment, including a micellar environment. In some embodiments, a mixture of solvents is used, e.g. MeTHF and water. Where methanol is used, the reaction mixture may be precipitated out of, simplifying purification.

The coupling of X6a and F6 can be achieved using one or more catalysts, one or more ligands, one or more borylating agents, one or more bases, and/or one or more additives. The skilled person can use their common general knowledge to determine appropriate amounts of these reagents.

In one embodiment, a coupling reaction having excellent yield and minimal by-product formation is characterized by at least one of the following:
i) The catalyst or pre-catalyst is present in an amount of 0.1 mol % to 5 mol %, 0.25 mol % to 3 mol %, 0.5 mol % to 1.5 mol %, preferably 0.5 mol % or more preferably 1 mol % relative to the number of moles of F6 or X6a;
ii) The number of moles of ligand, if present, is twice or 3 times the number of moles of catalyst or pre-catalyst, preferably twice;
iii) The molar ratio of F6:X6a is from 2:1 to 1:2, or from 1.5:1 to 1:1.5, 1.2:1 to 1:1.2 or 1:1;
iv) the additive is optional and when present is in an amount of 2 to 5 molar equivalents relative to F6 or X6a; and/or
v) The base is in an amount of 2 to 5 molar equivalents, preferably in an amount of 2 to 3 molar equivalents, most preferably in an amount of 3 molar equivalent relative to the number of moles of F6 or X6a.

The coupling reaction may be characterized by any one of i) to v) above. The coupling reaction may be characterized by any two of i) to v) above. The coupling reaction may be characterized by any three of i) to v) above. The coupling reaction may be characterized by any four of i) to v) above. The coupling reaction may be characterized by all of i) to v) above.

The coupling reaction may be characterized by i) and ii) above. The coupling reaction may be characterized by i) and iii) above. The coupling reaction may be characterized by i) and iv) above. The coupling reaction may be characterized by i) and v) above. The coupling reaction may be characterized by ii) and iii) above. The coupling reaction may be characterized by ii) and iv) above. The coupling reaction may be characterized by ii) and v) above. The coupling reaction may be characterized by iii) and iv) above. The coupling reaction may be characterized by iii) and v) above. The coupling reaction may be characterized by iv) and v) above.

In one embodiment, a coupling reaction having good yield and minimal by-product formation is characterized by at least one of the following:
i) the catalyst and ligand are provided as a pre-catalyst-ligand complex which is Pd and X-Phos-2G in an amount of is 0.5 mol % to 2 mol % relative to the number of moles of F6 or X6a;
ii) the base is triethylamine in an amount of 2 to 5 molar equivalents, preferably 3 molar equivalents relative to F6 or X6a;
iii) the additive is ethylene glycol in an amount of 2 to 5 molar equivalents, preferably 3 molar equivalents relative to F6 or X6a;
iv) the reaction is conducted in an alcoholic solvent, preferably methanol; and
v) the temperature of the reaction is 30° C. to 70° C., preferably 40° C. to 50° C., more preferably 50° C.

The coupling reaction may be characterized by any one of i) to v) above. The coupling reaction may be characterized by any two of i) to v) above. The coupling reaction may be characterized by any three of i) to v) above. The coupling reaction may be characterized by any four of i) to v) above. The coupling reaction may be characterized by all of i) to v) above.

The coupling reaction may be characterized by i) and ii) above. The coupling reaction may be characterized by i) and iii) above. The coupling reaction may be characterized by i) and iv) above. The coupling reaction may be characterized by i) and v) above. The coupling reaction may be characterized by ii) and iii) above. The coupling reaction may be characterized by ii) and iv) above. The coupling reaction may be characterized by ii) and v) above. The coupling reaction may be characterized by iii) and iv) above. The coupling reaction may be characterized by iii) and v) above. The coupling reaction may be characterized by iv) and v) above.

In a one embodiment, a coupling reaction having excellent yield and minimal by-product formation is characterized by at least one of the following:
i) the catalyst is Pd(MeCN)₂Cl₂ in an amount of 0.25 mol % to 2 mol % relative to the number of moles of X6b, 0.25 mol % to 1.5 mol %, preferably 0.5 mol % or more preferably 1 mol % relative to the number of moles of X6b; (conversion of X6b to X6a is about 98%)
ii) the ligand is BuPPh2 in an amount of 0.5 mol % to 4% relative to the number of moles of X6b, preferably 1 mol % or 2 mol % relative to the number of moles of X6b; in particular the catalyst is Pd(MeCN)₂Cl₂, the ligand is ťBuPPh2 and the number of moles of tBuPPh₂ is twice the number of moles of Pd(MeCN)₂Cl₂;
iii) the base is KOH in an amount of 2 to 5 molar equivalents, preferably 3 molar equivalents relative to X6b;
iv) the reaction is conducted in MeTHF and water mixture; and
v) the temperature of the reaction is 30° C. to 70° C., preferably 60° C.

The coupling reaction may be characterized by any one of i) to v) above. The coupling reaction may be characterized by any two of i) to v) above. The coupling reaction may be characterized by any three of i) to v) above. The coupling reaction may be characterized by any four of i) to v) above. The coupling reaction may be characterized by all of i) to v) above.

The coupling reaction may be characterized by i) and ii) above. The coupling reaction may be characterized by i) and iii) above. The coupling reaction may be characterized by i) and iv) above. The coupling reaction may be characterized by i) and v) above. The coupling reaction may be characterized by ii) and iii) above. The coupling reaction may be characterized by ii) and iv) above. The coupling reaction may be characterized by ii) and v) above. The coupling reaction may be characterized by iii) and iv) above. The coupling reaction may be characterized by iii) and v) above. The coupling reaction may be characterized by iv) and v) above.

In a one embodiment, the borylation of X6b to X6a and the cross-coupling of X6a and F6 are conducted in a one-pot reaction.

### Preparation of X6b

X6b is a key intermediate in the novel synthesis described herein. Thus, the invention further provides a synthetic intermediate, X6b:

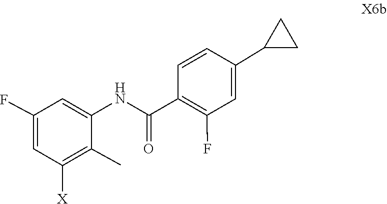

wherein X is F, Cl, Br, or I. in one embodiment, X is Br.

X6b can itself be synthesized in any suitable way. The invention provides methods of preparing synthetic intermediate X6b:

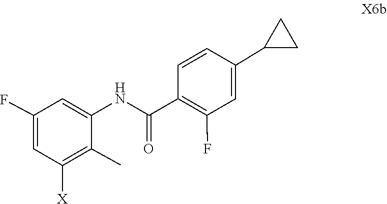

wherein X is F, Cl, Br, or I. In one embodiment X is Br.

In some embodiments, the method comprises reacting compound X6d with compound N6a:

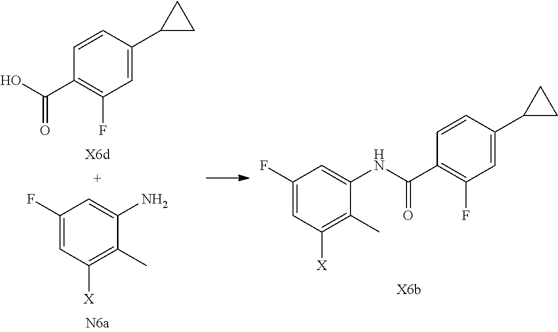

wherein X is Cl, Br, or I. In one embodiment, X is Br.

Carboxylic acid coupling reactions, including amidation reactions, are well-known to the skilled person, and typically involve reacting an amine with a carboxylic acid under coupling conditions, or converting the carboxylic acid group to an activated group that can react with an amine more easily.

Thus, in one embodiment, the synthesis of X6b involves using converting the carboxylic acid group of X6d into an activated carboxylic acid group. For example, the method can include conversion of compound X6d into compound X6c:

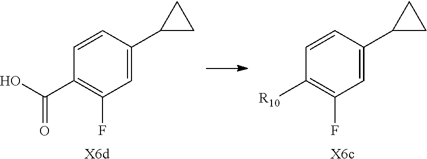

wherein R₁₀ is an activated carboxylic acid group, for example an acyl anhydride, acyl halide, or acyl phosphate, and wherein X is Cl, Br, or I. For example, conversion of X6d to the corresponding acyl chloride can be achieved using, thionyl chloride. The solvent may be an aromatic solvent such as toluene. The base may be pyridine. X6c can then be reacted with N6a to form compound X6b. These reactions can be conducted as a one-pot synthesis or sequentially. The formation of N6a from N6b may also be tied into this one-pot synthesis, such that X6c and N6a are prepared separately but then coupled.

Alternatively, X6b is prepared directly from X6d and N6a by employing a carboxylic acid activating reagent. Carboxylic acid activating reagents are well known, and include HBT, HATU, HBTU, TBTU, HOBt, PyAOP, HCTU, PyClock, TFFH, Carbodiimides (e.g. DCC), Carbonyl diimidazole (CDI), and Phosphonium salts (e.g. BOP, PyBOP).

The coupling of X6d or X6c and N6a can be conducted in the presence of a base, e.g. a tertiary alkyl amine base such as triethylamine or DIPEA, or an aryl amine base such as pyridine. The coupling of X6d or X6c and N6a can be conducted in isopropylacetate, toluene, or a mixture thereof.

X6d can be prepared from X6e:

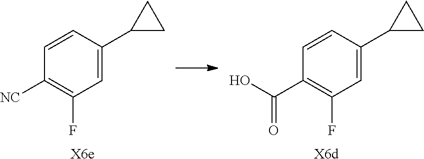

In one embodiment, X6d is prepared by contacting X6e with base, for example sodium hydroxide, which converts the cyano group to a carboxylic acid group.

X6e can be prepared from X6f:

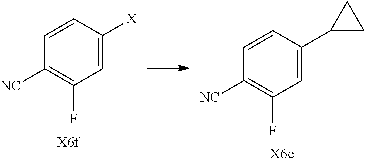

wherein X is Cl, Br, or I.

X6e is prepared by contacting X6f with X6g under cross-coupling conditions:

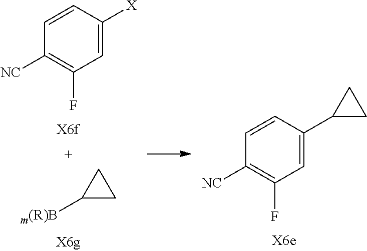

wherein X is F, Cl, Br, or I, m is 2 or 3 and R is F, Cl, Br, or I, OH, OC₁-C₆ alkyl, N(C₁-C₆ alkyl)₂, aryl, or wherein two or three R groups other than F, Cl, Br, I, or OH can be taken together to form a cyclic boronate ester, for example pinacol boronate, or N-methyliminodiacetic acid (MIDA) boronate.

Coupling of organoboron and aryl halide compounds is described above in relation to the coupling of X6b and F7, and similar conditions can be used for the formation of X6e.

X6f can be prepared from X6h:

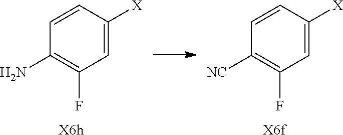

wherein X is Cl, Br, or I.

X6f can be prepared by diazotizing X6h, for example with nitrous acid or sodium nitrite under acidic conditions, followed by cyanation of the diazonium compound, for example using CuCN and/or NaCN.

X6h can be prepared from X6i:

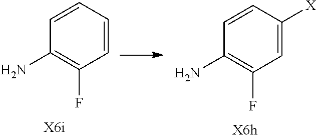

X6h can be prepared by contacting X6i with a halogenating agent, for example a chlorinating agent such as AlCl₃, or N-chlorosuccinimide, a brominating agent selected from the group consisting of N-bromosuccinate, 1,3-Dibromo-5,5-Dimethylhydantoin (DBDMH), N-bromosuccinimide, TBAB, phosphorus tribromide, bromine chloride, aluminium tribromide, Br₂ and FeBr₃, HBr, tribromoisocyanuric acid, ammonium bromide with ozone, TBBDA, and combinations thereof, or an iodinating reagent such as N-iodosuccinimide. X6h can also be prepared via a Sandmeyer reaction.

### Preparation of N6a

N6a is used in the preparation of X6b. therefore, the invention further provides the preparation of N6a. N6a can be prepared from N6b:

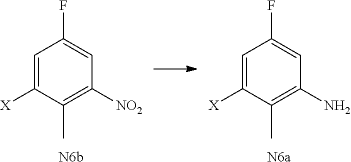

wherein Y is Cl, Br, or I.

N6a can be prepared by contacting N6b with a reducing agent, for example a reducing agent selected from the group consisting of: H₂ and Pt(V)/C; Raney nickel catalyst and H₂; Urushibara nickel catalyst and H₂; Adams' catalyst (PtO₂) and H₂; TiCl₃ and H₂; HCl and iron; NH₄Cl and iron; HCl and SnCl₂; samarium and NH₄Cl; FeCl₃, hydrazine hydrate; sodium hydrosulphite; hydrogen sulfide and base; hydroiodic acid; 1,3-dimethyl-2-imidazolidinone and sodium triethylsilanethiolate; and combinations thereof. In some embodiments, this reaction is conducted under micellar conditions.

N6b can be prepared from N6c:

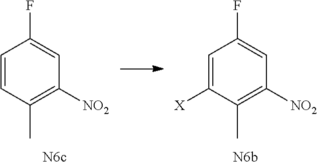

N6b can be prepared by contacting X6h with a halogenating agent for example a chlorinating agent such as AlCl₃, or N-chlorosuccinimide, brominating agent selected from the group consisting of N-bromosuccinate, N-bromosuccinimide, 1,3-Dibromo-5,5-Dimethylhydantoin (DBDMH), TBAB, phosphorus tribromide, bromine chloride, aluminium tribromide, Br₂ and FeBr₃, HBr, tribromoisocyanuric acid, ammonium bromide with ozone, TBBDA, and combinations thereof, or an iodinating reagent such as N-iodosuccinimide. X6h can also be prepared via a Sandmeyer reaction.

N6c can be prepared from N6d:

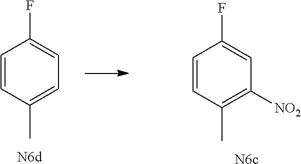

N6c can be prepared by contacting N6d with a nitrating agent, for example a nitrating agent selected from the group consisting of: nitric acid and sulfuric acid; nitric acid and acetic anhydride; tetrachloromethane, nitric acid and phosphorus pentoxide; isopentyl nitrate, trifluoromethanesulfonic acid, and 1-ethyl-3-methylimidazolium triflate; H-beta zeolite catalyst and N205; acetyl nitrate; and combinations thereof.

N6d can be prepared from N6e:

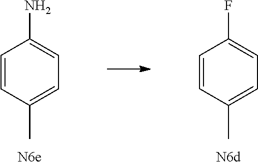

N6d can be prepared by contacting N6e with a diazotizing agent, such as nitrous acid or sodium nitrite under acidic conditions, followed by a fluorinating agent such as HF.

### Preparation of F6

F6 is used in the preparation of F₇ and can itself be prepared by any suitable method. Therefore, the invention further provides the method of preparation of F₆>F6 is prepared from F2 and F3:

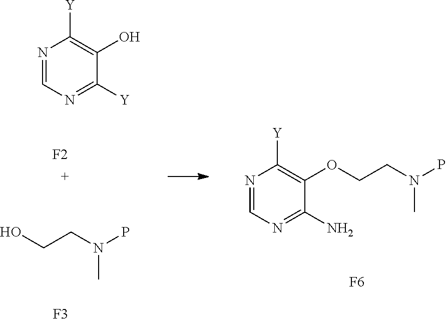

wherein Y is independently Cl, Br, or I.

In some embodiments, the preparation of F6 comprises reacting compound F2 with compound F3 to give compound F4:

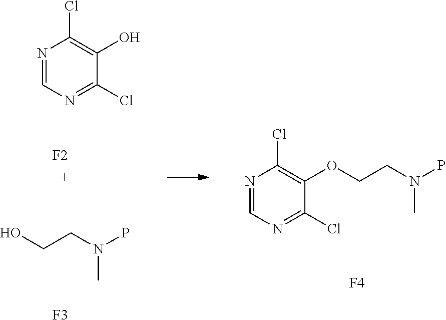

The reaction of F2 and F3 can be conducted under Mitsunobu conditions, for example in the presence of a phosphine compound such as PPh₃ (optionally on a resin support) and an azodicarbocylate such a DIAD or DEAD. In one embodiment, the reaction is conducted in an aromatic solvent such as toluene. In one embodiment, the solvent is dried to have a water content of less than 0.5 wt %, for example 0.1 wt %.

The preparation of F6 may comprise converting F4 to F6:

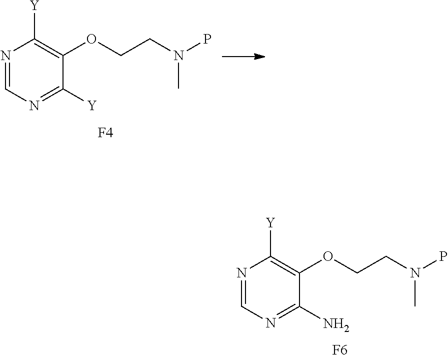

The conversion of F4 to F6 may be conducted using any suitable aminating reagent, for example ammonium hydroxide or water and ammonia. In one embodiment, the solvent is an alcoholic solvent such as iPrOH.

The reaction of F2 with F3 to give F4 and the conversion of F4 to compound F6 may be conducted in a sequential reaction or in a one-pot reaction.

Alternatively, F2 can be converted to F2′, via amination. Amination reagents include water and ammonia, or ammonium hydroxide, and this reaction may be conducted in a polar solvent such as an alcoholic solvent such as iPrOH. F2′ can then be reacted with F3, optionally under Mitsunobu conditions, for example in the presence of a phosphine compound such as PPh₃ and an azodicarbocylate such a DIAD or DEAD, to give F6:

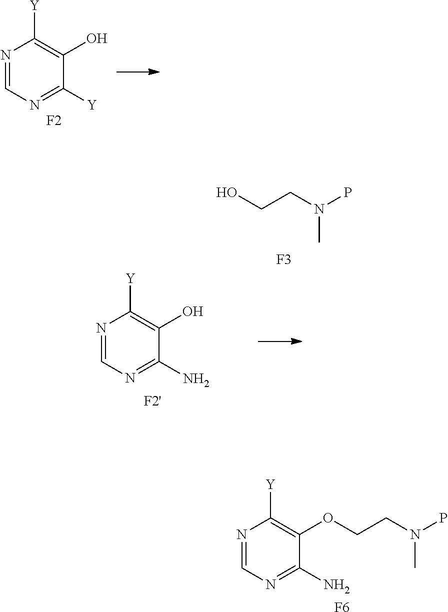

These reactions can be conducted in a sequential or in a one-pot fashion.

### Products Prepared According to Processes Described Herein and Uses Thereof

In one aspect, the invention provides synthetic routes to the LOU064 drug substance substantially pure of a nitrosamine, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide. Hence, the protection afforded by patents arising from the present application may extends to the direct product of the processes herein, which is remibrutinib drug substance substantially pure of nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide.

In another aspect, the invention comprises LOU064 drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide. For example the amount of N-(3-(6-amino-5-(2(methyl)(nitroso)amino)ethoxy)pyrimidin-4-yl)-5-fluoro-2-methylphenyl)-4-cyclopropyl-2-fluorobenzamide is less than about 1000 ppb, less than about 550 ppb, e.g. less than about 530 ppb; less than about 400 ppb, e.g. less than about 360 ppb; less than about 300 ppb; less than about 200 ppb; less than about 150 ppb, e.g. less than about 130 ppb; less than about 100 ppb, e.g. less than about 90 ppb; less than about 50 ppb or less than about 25 ppb

Furthermore, the invention comprises LOU064 drug substance prepared or preparable by a process described herein which does not involve INT 3 at any stage. Thus, in one embodiment, the remibrutinib drug substance prepared or preparable by a process described herein is also substantially free from INT 3 (5-fluoro-2-methyl-3-(4,4,5,5-tetramethyl-1,3,2-dioxaborolan-2-yl)aniline). For example, the amount of INT 3 may be less than about 100 ppm (parts per million), less than about 10 ppm, less than about 1 ppm, less than about 100 ppb (parts per billion), less than about 10 ppb, or less than about 1 ppb. In one embodiment, the remibrutinib prepared or preparable by a process described herein contains no INT 3 (5-fluoro-2-methyl-3-(4,4,5,5-tetramethyl-1,3,2-dioxaborolan-2-yl)aniline). Alternatively or in addition, remibrutinib drug substance prepared or preparable by a process described herein is substantially free from (3-amino-5-fluoro-2-methylphenyl) boronic acid. For example, the amount of (3-amino-5-fluoro-2-methylphenyl) boronic acid may be less than about 100 ppm (parts per million), less than about 10 ppm, less than about 1 ppm, less than about 100 ppb (parts per billion), less than about 10 ppb, or less than about 1 ppb. In one embodiment, the remibrutinib prepared or preparable by a process described herein contains no 3-amino-5-fluoro-2-methylphenyl) boronic acid.

### Pharmaceutical Composition of the Invention

In one aspect, the invention also comprises a pharmaceutical composition comprising remibrutinib drug substance prepared by or preparable by a process described herein, and thus may be substantially free from INT 3.

In another aspect, the invention also comprises a pharmaceutical composition comprising remibrutinib drug substance prepared by or preparable by a process described herein, and thus may be substantially free of a nitrosamine, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide. In one embodiment, the composition also contains at least one pharmaceutically acceptable excipient, and often contains at least two or more pharmaceutically acceptable excipients. Some suitable excipients are disclosed herein. Other excipients may be used that are known in the art without departing from the intent and scope of the present application.

As used herein, the term “pharmaceutically acceptable excipients” includes any and all solvents, carriers, diluents, dispersion media, coatings, surfactants, antioxidants, preservatives (e.g., antibacterial agents, antifungal agents, antioxidants), isotonic agents, absorption delaying agents, salts, drug stabilizers, binders, additives, bulking agents, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). It should be understood that unless a conventional excipient is incompatible with the active ingredient, the use of any conventional excipient in any therapeutic or pharmaceutical compositions is contemplated by the present application.

The pharmaceutical composition can be formulated for particular routes of administration such as oral administration, parenteral administration, and rectal administration, etc. In addition, the pharmaceutical compositions described herein can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, carriers or buffering agents, as well as adjuvants, such as solvents, preservatives, stabilizers, wetting agents, emulsifiers and bulking agents, etc.

Typically, the pharmaceutical compositions are tablets or capsules comprising the active ingredient together with at least one excipient, such as:
a) diluents, e.g., lactose, dextrose, sucrose, mannitol, sorbitol, cellulose and/or glycine;
b) lubricants, e.g., silica, talcum, stearic acid, its magnesium or calcium salt, sodium stearyl fumarate and/or polyethyleneglycol; for tablets also
c) binders, e.g., magnesium aluminum silicate, starch paste, gelatin, tragacanth, methylcellulose, sodium carboxymethylcellulose, polyvinylpyrrolidone and/or, polyvinylpyrrolidone vinyl acetate copolymer; if desired;
d) carriers such as an aqueous vehicle containing a co-solvating material such as captisol, PEG, glycerin, cyclodextrin, or the like;
e) disintegrants, e.g., starches, agar, alginic acid or its sodium salt, croscarmellose sodium, crospovidone, sodium starch glycolate, or effervescent mixtures; and/or
f) absorbents, colorants, flavors and sweeteners.

Tablets may be either film coated or enteric coated according to methods known in the art. In one embodiment, the compound or composition is prepared for oral administration, such as a tablet or capsule, for example, and optionally packaged in a multi-dose format suitable for storing and/or dispensing unit doses of a pharmaceutical product. Examples of suitable packaging include, but are not limited to, hermetically sealed foils, unit dose containers (e. g., vials), blister packs, and strip packs.

Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The present invention further comprises anhydrous pharmaceutical compositions and dosage forms comprising the remibrutinib prepared by or preparable by the methods described herein as active ingredients, since water may facilitate the degradation of certain compounds. Anhydrous pharmaceutical compositions and dosage forms can be prepared using anhydrous or low moisture containing ingredients and low moisture or low humidity conditions. An anhydrous pharmaceutical composition may be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The invention further comprises pharmaceutical compositions and dosage forms that comprise one or more agents that reduce the rate by which the compound described herein as an active ingredient will decompose. Such agents, which are referred to herein as “stabilizers,” include, but are not limited to, antioxidants such as ascorbic acid, pH buffers, or salt buffers, etc.

In some embodiment, remibrutinib which is substantially free of nitrosamine impurity can be provided in a pharmaceutical composition, dosage form as described in WO2022/162513 (Attorney docket number PAT059011-WO-PCT) which is hereby incorporated by reference in its entirety.

The pharmaceutical composition can be further formulated into a final dosage form. Example of dosage form is capsule or tablet. In one example, the dosage form is a film coated tablet, e.g. as disclosed in example 8 of WO2022/162513.

In one aspect, the pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 10-50 mg of active ingredients. In an embodiment, the pharmaceutical composition or combination of the present invention can be in unit dosage of about 10 mg, about 25 mg or about 50 mg. The therapeutically effective dosage or amount of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The drug substance (i.e. remibrutinib drug substance which is substantially free of nitrosamine impurity) may be present in the pharmaceutical composition (e.g. film coated tablet) in an amount of about 10 mg, about 15 mg, about 20 mg, about 25 mg, about 50 mg or about 100 mg.

In one aspect, the invention relates to a pharmaceutical composition comprising LOU064 drug substance substantially free of a nitrosamine, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, according to any one of the preceding claims and one or more pharmaceutically acceptable excipient(s).

In another aspect, the invention relates to a pharmaceutical composition comprising LOU064, or a pharmaceutically acceptable salt thereof, and one of more pharmaceutical acceptable excipients, and wherein the pharmaceutical composition is substantially free of a nitrosamine, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide. Content of nitrosamine impurity in the pharmaceutical composition are as defined above.

In another aspect, the invention relates to a final dosage form comprising a pharmaceutical composition as described above and one or more pharmaceutically acceptable excipient(s). In one embodiment the final dosage form is a film coated tablet. In another embodiment, the final dosage form in a film coated tablet wherein LOU064 is present in an amount of about 5 mg, about 10 mg, about 15 mg, about 20 mg, about 25 mg or about 100 mg, e.g. about 25 mg or about 100 mg.

In an embodiment, the drug substance (i.e. remibrutinib drug substance which is substantially free of nitrosamine impurity (e.g. impurity N-(3-(6-amino-5-(2(methyl)(nitroso)amino)ethoxy)pyrimidin-4-yl)-5-fluoro-2-methylphenyl)-4-cyclopropyl-2-fluorobenzamide) is present in said pharmaceutical composition (e.g. film coated tablet) in an amount of 25 mg. In one aspect of this embodiment, the content of nitrosamine (e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide) in remibrutinib drug substance, or in the composition is less than less than about 550 ppb, e.g. less than about 530 ppb; less than about 400 ppb, e.g. less than about 360 ppb; less than about 300 ppb; less than about 200 ppb; less than about 150 ppb, e.g. less than about 130 ppb; less than about 100 ppb, e.g. less than about 90 ppb; less than about 50 ppb or less than about 25 ppb, all relative to the total amount of LOU064 in free or salt form. In another aspect of this embodiment, the level of nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide, in remibrutinib drug substance or in the composition is between about 25 ppb and about 550 ppb, e.g., between about 25 ppb and about 530 ppb; or between about 25 ppb and about 400 ppb, e.g. between about 25 ppb and about 360 ppb; or between about 25 ppb and about 300 ppb; or between about 25 ppb and about 200 ppb, e.g. between about 25 ppb and about 90 ppb; or between about 25 ppb and about 100 ppb, e.g. between about 25 ppb and about 90 ppb, all relative to the total amount of LOU064 in free or salt form. In yet another aspect, the level of nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide, in remibrutinib drug substance or in composition is between about 100 ppb and about 550 ppb, e.g. between about 100 ppb and about 530 ppb; or is between about 100 ppb and about 400 ppb e.g. between about 100 ppb and about 360 ppb; or is between about 100 ppb and about 350 ppb, e.g. between about 100 ppb and about 320 ppb; is between about 100 ppb and about 250 ppb; or is between about 100 ppb and about 150 ppb, e.g. between about 100 ppb and about 130 ppb, all relative to the total amount of LOU064 in free or salt form

In another embodiment, the drug substance (i.e. remibrutinib drug substance which is substantially free of nitrosamine impurity) is present in the pharmaceutical composition (e.g. film coated tablet) in an amount of 100 mg. In one aspect of this embodiment, the content of nitrosamine (e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide) in remibrutinib drug substance or in the composition is less than less than about 550 ppb, e.g. less than less than about 130 ppb; less than about 100 ppb, e.g. less than about 90 ppb; less than about 50 ppb or less than about 25 ppb, all relative to the total amount of LOU064 in free or salt form. In another aspect of this embodiment, the level of nitrosamine, e.g. N-(3-(6-amino-5-(2(methyl)(nitroso)amino)ethoxy)pyrimidin-4-yl)-5-fluoro-2-methylphenyl)-4-cyclopropyl-2-fluorobenzamide, in remibrutinib drug substance or in composition is between about 25 ppb and about 130 ppb, or between about 35 ppb and about 90 ppb; or between about 25 ppb and about 100 ppb, e.g. between about 25 ppb and about 90 ppb, or between about 100 ppb and about 130 ppb, all relative to the total amount of LOU064 in free or salt form.

The above-cited dosage properties are demonstrable in vitro and in vivo tests using advantageously mammals, e.g., mice, rats, dogs, monkeys or isolated organs, tissues and preparations thereof. The compounds described herein can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg. In an embodiment, the therapeutically effective amount in vivo ranges between about 10 mg to about 200 mg daily, for example, about 10 mg, about 20 mg, about 25 mg, about 35 mg, about 50 mg, about 100 mg or about 200 mg daily. In an embodiment, the therapeutically effective amount in vivo is selected from about 10 mg, about 35 mg, about 50 mg or about 100 mg once a day. In an embodiment, the therapeutically effective amount in vivo is selected from about 10 mg, about 25 mg, about 50 mg or about 100 mg twice a day.

### Manufacture of the Pharmaceutical Composition (Drug Product)

The invention further provides methods of preparing a pharmaceutical composition comprising (i) remibrutinib or a pharmaceutically acceptable salt thereof and (ii) one or more pharmaceutically acceptable excipients, wherein the pharmaceutical composition is substantially free of nitrosamines, particularly N-(3-(6-amino-5-(2(methyl)(nitroso)amino)ethoxy)pyrimidin-4-yl)-5-fluoro-2-methylphenyl)-4-cyclopropyl-2-fluorobenzamide. While methods of preparing such pharmaceutical compositions are described herein it will be readily apparent to a person of ordinary skill in the art that two or more of these methods may also be used in combination.

Thus, the present invention also provides the combined methods of preparing a pharmaceutical composition comprising (i) remibrutinib or a pharmaceutically acceptable salt thereof and (ii) one or more pharmaceutically acceptable excipients, wherein the pharmaceutical composition is substantially free of nitrosamines, particularly N-(3-(6-amino-5-(2(methyl)(nitroso)amino)ethoxy)pyrimidin-4-yl)-5-fluoro-2-methylphenyl)-4-cyclopropyl-2-fluorobenzamide. The pharmaceutical compositions produced by these methods may be used in the pharmaceutical products described herein.

Thus, an aspect of the invention relates to a method of preparing a pharmaceutical composition comprising mixing remibrutinib or a pharmaceutically acceptable salt thereof with one or more pharmaceutically acceptable excipients, wherein the one or more pharmaceutically acceptable excipients, and optionally each of the one or more pharmaceutically acceptable excipients, has a content of nitrites of less than 1.5 ppm, less than 1 ppm, or less than about 0.5 ppm. The one or more pharmaceutically acceptable excipients, e.g. each of the one or more pharmaceutically acceptable excipients, may have a content of nitrites of less than about 0.4 ppm, about 0.3 ppm, about 0.2 ppm, or about 0.1 ppm.

Accordingly, the invention provides a method of preparing a pharmaceutical composition comprising mixing remibrutinib or a pharmaceutically acceptable salt thereof with one or more pharmaceutically acceptable excipients, wherein each of the one or more pharmaceutically acceptable excipients has a content of nitrites of less than 1.5 ppm, less than 1 ppm, or less than about 0.5 ppm, relative to the amount of the respective excipient. Each of the one or more pharmaceutically acceptable excipients may have a content of nitrites of less than about 0.4 ppm, about 0.3 ppm, about 0.2 ppm, or about 0.1 ppm, relative to the amount of the respective excipient.

Another aspect of the invention relates to a method of preparing a pharmaceutical composition comprising mixing remibrutinib or a pharmaceutically acceptable salt thereof with more than one pharmaceutically acceptable excipient, wherein the combined excipients have a total content of nitrites of less than 1.5 ppm, less than 1 ppm, or less than about 0.5 ppm relative to the combined amount of the excipients. The combined excipients may have a total content of nitrites of less than about 0.4 ppm, about 0.3 ppm, about 0.2 ppm, or about 0.1 ppm, relative to the combined amount of the excipients.

The method of preparing a pharmaceutical composition comprising remibrutinib or a pharmaceutically acceptable salt thereof, and one or more pharmaceutically acceptable excipients, may comprise a step of mixing remibrutinib or said pharmaceutically acceptable salt thereof with the one or more pharmaceutically acceptable excipients. The method may further comprise a step of drying the resulting mixture. Thus, an aspect of the invention relates to a method of preparing a pharmaceutical composition comprising (i) remibrutinib or a pharmaceutically acceptable salt thereof; and (ii) one or more pharmaceutically acceptable excipients, wherein the method comprises: (a) mixing remibrutinib or said pharmaceutically acceptable salt thereof with the one or more excipients; and (b) drying the resulting mixture. The drying step may be carried out until the water activity value becomes less than 0.12, less than 0.10, less than 0.09 or less than 0.08. The method may further comprise, either before or after the step of drying, a step of processing the composition into a solid oral dosage form. For example, the method may comprise a further step of compressing the composition into a tablet. Alternatively, the method may comprise a further step of filling the composition into a capsule. The method may comprise, after the step of drying, a further step of storing the composition in the presence of a desiccant, e.g. storing the composition in a sealed container which also contains a desiccant or storing the composition in a sealed pharmaceutical package which also contains a desiccant in a separate container.

The method of preparing a pharmaceutical composition comprising remibrutinib or a pharmaceutically acceptable salt thereof, and one or more pharmaceutically acceptable excipients, may comprise one or more steps as described in WO2022/162513 (Attorney docket number PAT059011-WO-PCT) which is hereby incorporated by reference in its entirety.

In one embodiment, the one or more pharmaceutically acceptable excipients may be selected from the group consisting of lactose, microcrystalline cellulose, mannitol, sucrose, starch, granulated hydrophilic fumed silica, polyvinylpyrrolidone-vinyl acetate copolymer, polyvinyl pyrrolidone, hydroxypropyl cellulose, hydroxypropylmethyl cellulose, hypromellose, carboxymethyl cellulose, methyl cellulose, hydroxyethyl cellulose, carboxyethyl cellulose, carboxymethylhydroxyethyl cellulose, polyethylene glycol, polyvinylalcohol, shellac, polyvinyl alcohol-polyethylene glycol co-polymer, polyethylene-propylene glycol copolymer, sodium lauryl sulfate, potassium lauryl sulfate, ammonium lauryl sulfate, sodium lauryl ether sulfate, polysorbates, perfluorobutanesulfonate, dioctyl sulfosuccinate, magnesium carbonate, kaolin, cellulose (e.g. microcrystalline cellulose, powdered cellulose), calcium phosphate, or sodium phosphate, croscarmellose sodium, crospovidone, sodium starch glycolate, corn starch, or alginic acid, magnesium stearate, sodium stearyl fumarate, stearic acid and talc.

In an aspect of the above embodiment, the one or more pharmaceutically acceptable excipients may be selected from the group consisting of magnesium stearate, sodium stearyl fumarate, microcrystalline cellulose, crospovidone, croscamellose sodium, lactose, mannitol, sodium lauryl sulfate, and polyvinylpyrrolidone-vinyl acetate copolymer (copovidone).

The one or more pharmaceutically acceptable excipients may be or may comprise magnesium stearate or sodium stearyl fumarate. The content of nitrites in the magnesium stearate or sodium stearyl fumarate may be less about 1.5 ppm, optionally less than about 1 ppm, less than about 0.5 ppm, less than about 0.2 ppm.

The one or more pharmaceutically acceptable excipients may be or may comprise microcrystalline cellulose. The content of nitrites in the microcrystalline cellulose may be less than about 500 ppb, less than about 400 ppb, less than about 300 ppb, less than about 200 ppb, less than about 100 ppb, less than about 100 ppb, less than about 90 ppb, less than about 80 ppb, less than about 70 ppb, less than about 60 ppb, less than about 50 ppb, less than about 40 ppb, less than about 30 ppb, less than about 20 ppb, or less than about 10 ppb. Preferably, the content of nitrites in the microcrystalline cellulose may be less than about 100 ppb. Suitable microcrystalline cellulose excipients are known to those skilled in the art and include types MCC PH102 and MCC PH105. The microcrystalline cellulose may be MCC PH102.

The one or more pharmaceutically acceptable excipients may be or may comprise sodium lauryl sulfate (SLS). The content of nitrites in SLS is less than about 1.5 ppm, less than about 1 ppm, less than about 0.5 ppm, optionally no more than about 500 ppb, no more than about 200 ppb, no more than about 100 ppb.

The one or more pharmaceutically acceptable excipients may be or may comprise polyvinylpyrrolidone-vinyl acetate copolymer (copovidone). The content of nitrites in copovidone may be less about 500 ppb, less than about 400 ppb, less than about 300 ppb, less than about 200 ppb, or less than about 100 ppb.

As disclosed elsewhere herein, the amount of nitrites in a composition, e.g. the amount of nitrites in a pharmaceutically acceptable excipient, may be determined using the Griess test (e.g. example 16). Thus, the invention provides a method of preparing a composition comprising (i) remibrutinib or a pharmaceutically acceptable salt thereof and (ii) one or more pharmaceutically acceptable excipients, wherein the method comprises the use of the Griess test to determine the amount of nitrites in one, a plurality, or each of the one or more excipients, optionally wherein the one or more excipients in which the amount of nitrites is determined is selected from the group consisting of magnesium stearate, sodium stearyl fumarate, microcrystalline cellulose, crospovidone, croscamellose sodium, lactose, mannitol, sodium lauryl sulfate, and polyvinylpyrrolidone-vinyl acetate copolymer (copovidone).

The remibrutinib or pharmaceutically acceptable salt thereof that is used in the methods of preparing a pharmaceutical composition may be prepared by the methods of preparing remibrutinib or a pharmaceutically acceptable salt thereof that are described elsewhere herein.

### Testing of the Drug Substance and Pharmaceutical Composition (Drug Product)

The invention further provides methods of testing the remibrutinib or pharmaceutically acceptable salt thereof, as well as methods of testing the pharmaceutical compositions comprising (i) remibrutinib or a pharmaceutically acceptable salt thereof and (ii) one or more pharmaceutically acceptable excipients, for the presence and/or amount of nitrosamines, in particular N-(3-(6-amino-5-(2(methyl)(nitroso)amino)ethoxy)pyrimidin-4-yl)-5-fluoro-2-methylphenyl)-4-cyclopropyl-2-fluorobenzamide. Thus, an aspect of the invention relates to a method of evaluating a composition comprising remibrutinib or a pharmaceutically acceptable salt thereof, the method comprising testing the composition for the presence and/or amount of nitrosamines, in particular in N-(3-(6-amino-5-(2(methyl)(nitroso)amino)ethoxy)pyrimidin-4-yl)-5-fluoro-2-methylphenyl)-4-cyclopropyl-2-fluorobenzamide.

The method may be used to validate a process for the production of a composition comprising remibrutinib or a pharmaceutically acceptable salt thereof. Thus, an aspect of the invention relates to a method of validating a process for the production of a composition comprising remibrutinib or a pharmaceutically acceptable salt thereof, the method comprising testing the composition produced by said process for the presence and/or amount of nitrosamines, in particular N-(3-(6-amino-5-(2(methyl)(nitroso)amino)ethoxy)pyrimidin-4-yl)-5-fluoro-2-methylphenyl)-4-cyclopropyl-2-fluorobenzamide.

Another aspect of the invention relates to a method of obtaining regulatory approval for a pharmaceutical composition which comprises or consists of remibrutinib or a pharmaceutically acceptable salt thereof, wherein the method comprises (i) testing the composition for the presence and/or amount of nitrosamines, in particular N-(3-(6-amino-5-(2(methyl)(nitroso)amino)ethoxy)pyrimidin-4-yl)-5-fluoro-2-methylphenyl)-4-cyclopropyl-2-fluorobenzamide, and (ii) submitting the results of said testing to a regulatory authority. Suitable regulatory authorities to which the results may be submitted are, e.g. the FDA, the EMA, the MHRA, Swissmedic, or the PMDA.

In some aspects of the methods of the invention, a batch of the composition is tested to determine the presence and/or amount of nitrosamines in said batch, in particular the presence and/or total amount of N-(3-(6-amino-5-(2(methyl)(nitroso)amino)ethoxy)pyrimidin-4-yl)-5-fluoro-2-methylphenyl)-4-cyclopropyl-2-fluorobenzamide. In particular, a sample of the batch is tested. The batch testing may be used to determine whether to prepare a pharmaceutical product from said batch. For example, a pharmaceutical product may be prepared from the batch only if the batch is determined to have a total amount of nitrosamines of less than less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb, relative to remibrutinib in free or salt form.

Thus, an aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of remibrutinib or of a pharmaceutically acceptable salt thereof;
b. determining the total amount of nitrosamines in said batch, in particular by testing a sample of the batch; and
c. preparing the pharmaceutical product from the batch only if the batch is determined to have a total amount of nitrosamines of less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb, relative to the amount of remibrutinib in free or salt form.

In some aspects, a pharmaceutical product may be prepared from the batch only if the batch is determined to have a total amount of nitrosamines (e.g. N-(3-(6-amino-5-(2(methyl)(nitroso)amino)ethoxy)pyrimidin-4-yl)-5-fluoro-2-methylphenyl)-4-cyclopropyl-2-fluorobenzamide) which would result in an amount of said nitrosamines that corresponds to an amount of no more than 100 ng/day of nitrosamine free base (such as no more than 100 ng/day of N-(3-(6-amino-5-(2(methyl)(nitroso)amino)ethoxy)pyrimidin-4-yl)-5-fluoro-2-methylphenyl)-4-cyclopropyl-2-fluorobenzamide) being administered to the patient when the composition is administered according to an approved dosage regimen of the remibrutinib or pharmaceutically acceptable salt thereof. Thus, an aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of remibrutinib or of a pharmaceutically acceptable salt thereof;
b. determining the total amount of nitrosamines (e.g. N-(3-(6-amino-5-(2(methyl)(nitroso)amino)ethoxy)pyrimidin-4-yl)-5-fluoro-2-methylphenyl)-4-cyclopropyl-2-fluorobenzamide) in said batch, in particular by testing a sample of the batch; and
c. preparing the pharmaceutical product from the batch only if the batch is determined to have a total amount of nitrosamines (e.g. N-(3-(6-amino-5-(2(methyl)(nitroso)amino)ethoxy)pyrimidin-4-yl)-5-fluoro-2-methylphenyl)-4-cyclopropyl-2-fluorobenzamide) which would result in an amount of said nitrosamines (e.g. N-(3-(6-amino-5-(2(methyl)(nitroso)amino)ethoxy)pyrimidin-4-yl)-5-fluoro-2-methylphenyl)-4-cyclopropyl-2-fluorobenzamide) that corresponds to an amount of no more than 100 ng/day of nitrosamine (such as no more than 100 ng/day of N-(3-(6-amino-5-(2(methyl)(nitroso)amino)ethoxy)pyrimidin-4-yl)-5-fluoro-2-methylphenyl)-4-cyclopropyl-2-fluorobenzamide) being administered to the patient when the composition is administered according to an approved dosage regimen of the remibrutinib or pharmaceutically acceptable salt thereof.

In some aspects, a batch of the composition is tested to determine the total amount of N-(3-(6-amino-5-(2(methyl)(nitroso)amino)ethoxy)pyrimidin-4-yl)-5-fluoro-2-methylphenyl)-4-cyclopropyl-2-fluorobenzamide in said batch. In particular, a sample of the batch is tested. This batch testing may be used to determine whether to prepare a pharmaceutical product from said batch. For example, a pharmaceutical product may be prepared from the batch only if the batch is determined to have a total amount of N-(3-(6-amino-5-(2(methyl)(nitroso)amino)ethoxy)pyrimidin-4-yl)-5-fluoro-2-methylphenyl)-4-cyclopropyl-2-fluorobenzamide less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form. Thus, an aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of remibrutinib or of a pharmaceutically acceptable salt thereof;
b. determining the total amount of N-(3-(6-amino-5-(2(methyl)(nitroso)amino)ethoxy)pyrimidin-4-yl)-5-fluoro-2-methylphenyl)-4-cyclopropyl-2-fluorobenzamide in said batch, in particular by testing a sample of the batch; and
c. preparing the pharmaceutical product from the batch only if the batch is determined to have a total amount of N-(3-(6-amino-5-(2(methyl)(nitroso)amino)ethoxy)pyrimidin-4-yl)-5-fluoro-2-methylphenyl)-4-cyclopropyl-2-fluorobenzamide is less about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form.

In some aspects, a batch of the composition is tested to determine the amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, in said batch. In particular, a sample of the batch is tested. This batch testing may be used to determine whether to prepare a pharmaceutical product from said batch. For example, a pharmaceutical product may be prepared from the batch only if the batch is determined to have an amount of total nitrosamines, including N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, of less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form. Preferably, a pharmaceutical product may be prepared from the batch only if the batch is determined to have an amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines of less than about 360 ppb; less than about 150 ppb, e.g. less than about 130 ppb; less than about 100 ppb, e.g. less than about 90 ppb relative to the total amount of remibrutinib in free or salt form. Thus, an aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of remibrutinib or of a pharmaceutically acceptable salt thereof;
b. determining the amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, in said batch, in particular by testing a sample of the batch; and
c. preparing the pharmaceutical product from the batch only if the batch is determined to have an amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines of less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form.

In some aspects, a pharmaceutical product may be prepared from the batch only if the batch is determined to have an amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, which would result in an amount of said total nitrosamines that corresponds to an amount of no more than 100 ng/day of nitrosamine free base being administered to the patient when the composition is administered according to an approved dosage regimen of remibrutinib or pharmaceutically acceptable salt thereof. Thus, an aspect of the invention provides a process for preparing a pharmaceutical product comprising a composition that comprises: (i) remibrutinib or a pharmaceutically acceptable salt thereof; and (ii) one or more pharmaceutically acceptable excipients, the process comprising:
a. obtaining a batch of remibrutinib or of a pharmaceutically acceptable salt thereof;
b. determining the amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, in said batch, in particular by testing a sample of the batch; and
c. preparing the pharmaceutical product from the batch only if the batch is determined to have an amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, which would result in an amount of said total nitrosamines that corresponds to an amount of no more than 100 ng/day of nitrosamine free base being administered to the patient when the composition is administered according to an approved dosage regimen of remibrutinib or pharmaceutically acceptable salt thereof.

In some aspect, stability testing may be performed using a sample of a batch of the composition. Following this stability testing, the sample of the batch may be tested for the total amount of nitrosamines (such as N-(3-(6-amino-5-(2(methyl)(nitroso)amino)ethoxy)pyrimidin-4-yl)-5-fluoro-2-methylphenyl)-4-cyclopropyl-2-fluorobenzamide). This testing of the sample of the batch may be used to determine whether the batch is suitable for distribution and/or administration to a patient. For example, the batch may be determined to be suitable for distribution only if the sample of the batch after stability testing is determined to have a total amount of nitrosamines (such as N-(3-(6-amino-5-(2(methyl)(nitroso)amino)ethoxy)pyrimidin-4-yl)-5-fluoro-2-methylphenyl)-4-cyclopropyl-2-fluorobenzamide) of less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form.

Thus, an aspect of the invention provides a process of distributing a validated batch of a pharmaceutical product comprising a composition that comprises: (i) remibrutinib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the total amount of nitrosamines (such as N-(3-(6-amino-5-(2(methyl)(nitroso)amino)ethoxy)pyrimidin-4-yl)-5-fluoro-2-methylphenyl)-4-cyclopropyl-2-fluorobenzamide) in the sample of the batch after stability testing; and
d. validating the batch for distribution only if the sample of the batch after stability testing is determined to have a total amount of nitrosamines (such as N-(3-(6-amino-5-(2(methyl)(nitroso)amino)ethoxy)pyrimidin-4-yl)-5-fluoro-2-methylphenyl)-4-cyclopropyl-2-fluorobenzamide) of less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form.

In some aspects, stability testing may be performed using a sample of a batch of the composition and the sample of the batch may be tested for the total amount of N-(3-(6-amino-5-(2(methyl)(nitroso)amino)ethoxy)pyrimidin-4-yl)-5-fluoro-2-methylphenyl)-4-cyclopropyl-2-fluorobenzamide in said sample of the batch after stability testing. This testing of the sample of the batch may be used to determine whether the batch is suitable for distribution and/or administration to a patient. For example, the batch may be determined to be suitable for distribution only if the sample of the batch after stability testing is determined to have a total amount of N-(3-(6-amino-5-(2(methyl)(nitroso)amino)ethoxy)pyrimidin-4-yl)-5-fluoro-2-methylphenyl)-4-cyclopropyl-2-fluorobenzamide of less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form. Thus, an aspect of the invention provides a process of distributing a validated batch of a pharmaceutical product comprising a composition that comprises: (i) remibrutinib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the total amount of N-(3-(6-amino-5-(2(methyl)(nitroso)amino)ethoxy)pyrimidin-4-yl)-5-fluoro-2-methylphenyl)-4-cyclopropyl-2-fluorobenzamide in said sample of said batch after stability testing; and
d. validating the batch for distribution only if the sample of the batch after stability testing is determined to have a total amount of N-(3-(6-amino-5-(2(methyl)(nitroso)amino)ethoxy)pyrimidin-4-yl)-5-fluoro-2-methylphenyl)-4-cyclopropyl-2-fluorobenzamide of less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form.

In some aspects, stability testing may be performed using a sample of a batch of the composition and the sample of the batch may be tested for the amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, in said batch after stability testing. This testing of the sample of the batch may be used to determine whether the batch is suitable for distribution and/or administration to a patient. For example, the batch may be determined to be suitable for distribution only if the sample of the batch after stability testing is determined to have an amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, of less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form. Thus, an aspect of the invention provides a process of distributing a validated batch of a pharmaceutical product comprising a composition that comprises: (i) remibrutinib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, in said batch after stability testing; and
d. validating the batch for distribution only if the sample of the batch after stability testing is determined to have an amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, of less than about 1000 ppb (e.g. less than about 550 ppb, e.g. less than about 530 ppb; less than about 400 ppb, e.g. less than about 360 ppb; less than about 150 ppb, e.g. less than about 130 ppb; less than about 100 ppb, e.g. less than about 90 ppb); less than about 50 ppb; or less than about 25 ppb relative to the total amount of remibrutinib in free or salt form.

In some aspects, the batch may be determined to be suitable for distribution only if the sample of the batch after stability testing is determined to have an amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, which would result in an amount of said total nitrosamines which corresponds to an amount of no more than 100 ng/day of nitrosamine free base being administered to the patient when the composition is administered according to an approved dosage regimen of remibrutinib or pharmaceutically acceptable salt thereof. Thus, an aspect of the invention provides a process of distributing a validated batch of a pharmaceutical product comprising a composition that comprises: (i) remibrutinib or a pharmaceutically acceptable salt thereof; and (ii) one or more pharmaceutically acceptable excipients, the process comprising:
a. producing a batch of the pharmaceutical product;
b. performing stability testing with a sample of said batch;
c. determining the amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, in the sample of the batch after stability testing; and
d. validating the batch for distribution only if the sample of the batch after stability testing is determined to have an amount of total nitrosamines, including both N-(3-(6-amino-5-(2(methyl)(nitroso)amino)ethoxy)pyrimidin-4-yl)-5-fluoro-2-methylphenyl)-4-cyclopropyl-2-fluorobenzamide and other nitrosamines, which would result in an amount of said total nitrosamines which corresponds to an amount of no more than 100 ng/day of nitrosamine free base being administered to the patient when the composition is administered according to an approved dosage regimen of remibrutinib or pharmaceutically acceptable salt thereof.

In any of these methods, the step of testing for the presence and/or amount of nitrosamines may be performed using high performance liquid chromatography (HPLC)- and/or gas chromatography (GC)-mass spectroscopy. Likewise, the step of determining the total amount of N-(3-(6-amino-5-(2(methyl)(nitroso)amino)ethoxy)pyrimidin-4-yl)-5-fluoro-2-methylphenyl)-4-cyclopropyl-2-fluorobenzamide may be performed using high performance liquid chromatography (HPLC)- and/or gas chromatography (GC)-mass spectroscopy. For example, the HPLC-MS method performed may be the method provided in Example 15.

The invention also provides for the use of N-(3-(6-amino-5-(2(methyl)(nitroso)amino)ethoxy)pyrimidin-4-yl)-5-fluoro-2-methylphenyl)-4-cyclopropyl-2-fluorobenzamide as a reference standard to detect an impurity in a composition comprising remibrutinib or a pharmaceutically acceptable salt thereof. The composition may comprise remibrutinib or a pharmaceutically acceptable salt thereof and one or more pharmaceutically acceptable excipients. The impurity may be N-(3-(6-amino-5-(2(methyl)(nitroso)amino)ethoxy)pyrimidin-4-yl)-5-fluoro-2-methylphenyl)-4-cyclopropyl-2-fluorobenzamide. The detection of the impurity may be performed using high performance liquid chromatography (HPLC)- and/or gas chromatography (GC)-mass spectroscopy.

### Method of Treatment

The present invention also comprises a method for the treatment of disorders mediated by BTK or ameliorated by the inhibition of BTK, comprising administering to a patient in need of such treatment a therapeutically effective amount of remibrutinib drug substance prepared by or preparable by a method described herein (remibrutinib drug substance substantially free of N-(3-(6-amino-5-(2(methyl)(nitroso)amino)ethoxy)pyrimidin-4-yl)-5-fluoro-2-methylphenyl)-4-cyclopropyl-2-fluorobenzamide as described herein).

Also included in the invention are methods of treatment disorders mediated by BTK or ameliorated by the inhibition of BTK, comprising administering to a patient in need of such treatment, a pharmaceutical composition substantially free of N-(3-(6-amino-5-(2(methyl)(nitroso)amino)ethoxy)pyrimidin-4-yl)-5-fluoro-2-methylphenyl)-4-cyclopropyl-2-fluorobenzamide as described herein.

In another aspect, the present invention also comprises the use of remibrutinib prepared by or preparable by a method described herein (remibrutinib substantially free of the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide as described herein) for the preparation of a medicament for the treatment of disorders mediated by BTK or ameliorated by the inhibition of BTK.

In another aspect, the present invention also comprises remibrutinib drug substance prepared by or preparable by a method described herein (e.g. remibrutinib drug substance substantially free of the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide as described herein) for use in the treatment of disorders mediated by BTK or ameliorated by the inhibition of BTK.

Also included in the invention, are pharmaceutical composition substantially free of the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide as described herein, for use in the treatment of disorders mediated by BTK or ameliorated by the inhibition of BTK.

Remibrutinib drug substance prepared by or preparable by a method described herein (e.g. remibrutinib drug substance substantially free of a nitrosamine, (e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide as described herein) and pharmaceutical composition substantially free of a nitrosamine, (e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide as described herein are useful in the treatment of the following diseases or disorders mediated by BTK or ameliorated by inhibition of BTK: Autoimmune disorders, inflammatory diseases, allergic diseases, airway diseases, such as asthma and chronic obstructive pulmonary disease (COPD), transplant rejection; diseases in which antibody production, antigen presentation, cytokine production or lymphoid organogenesis are abnormal or are undesirable; including rheumatoid arthritis, systemic onset juvenile idiopathic arthritis (SOJIA), gout, pemphigus vulgaris, idiopathic thrombocytopenia purpura, systemic lupus erythematosus, multiple sclerosis, myasthenia gravis, Sjögren's syndrome, hidradenitis suppurativa, IgE driven allergy, e.g. drug, venom, food allergy; autoimmune hemolytic anemia, anti-neutrophil cytoplasmic antibodies (ANCA)-associated vasculitus, cryoglobulinemia, thrombotic thrombocytopenia purpura, chronic urticaria (chronic spontaneous urticaria, inducible urticaria), chronic allergy (atopic dermatitis, contact dermatitis, allergic rhinitis), atherosclerosis, type 1 diabetes, type 2 diabetes, inflammatory bowel disease, ulcerative colitis, morbus Crohn, pancreatitis, glomerolunephritis, Goodpasture's syndrome, Hashimoto's thyroiditis, Grave's disease, antibody-mediated transplant rejection (AMR), graft versus host disease, B cell-mediated hyperacute, acute and chronic transplant rejection; thromboembolic disorders, myocardial infarct, angina pectoris, stroke, ischemic disorders, pulmonary embolism; cancers of haematopoietic origin including, but not limited to, multiple myeloma; a leukaemia; acute myelogenous leukemia; chronic myelogenous leukemia; lymphocytic leukemia; myeloid leukemia; non-Hodgkin lymphoma; lymphomas; polycythemia vera; essential thrombocythemia; myelofibrosis with myeloid metaplasia; and Waldenstroem disease.

Remibrutinib drug substance prepared by or preparable by a method described herein (remibrutinib substantially free of the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide as described herein) is especially useful in the treatment of chronic urticaria, e.g. chronic spontaneous urticaria or chronic inducible urticaria; Sjögren's syndrome, multiple sclerosis, hidradenitis suppurativa and food allergy.

### EXAMPLES

### Example 1: Method of Determination of nitrosamine (N-(3-(6-amino-5-(2(methyl)(nitroso)amino)ethoxy)pyrimidin-4-yl)-5-fluoro-2-methylphenyl)-4-cyclopropyl-2-fluorobenzamide)

### Example 1a: Method of Determination of Nitrosamine in Intermediate F8

Limit-test by LC-MS (SIM mode), AJS-ES, positive mode, SIM Ion: 483.2 [M+H]+
Column: YMC-Triart C18, 100×3.0 mm, particle size 1.9 μm, 12 nm
Mobile phase A: Dissolve 0.315 g of Ammonium formate in 1000 ml of water, add 0.5 ml of formic acid
Mobile phase B: Methanol
Sample solvent: Methanol+0.2% (v/v) Formic acid
Sample solution: approx. 100 mg of the sample in 20 mL
Flow: 0.6 mL/min
Injection volume: 3 μL
Autosampler cooled, 8° C.
Column temperature. 80° C.
Gradient:

| Min | Mobile phase A (%) | Mobile phase B (%) |
|---|---|---|
| 0 | 65 | 35 |
| 1 | 65 | 35 |
| 11 | 10 | 90 |
| 12 | 10 | 90 |
| 12.1 | 65 | 35 |
| 16 | 65 | 35 |

1 ppb of nitrosamine in F8 refers to 1 part (mass) of nitrosamine in 10⁹ parts (mass) of F8 as isolated (i.e. F8 including potentially other impurities).

### Example 1b: Method of Determination of Nitrosamine in Drug Substance

Limit-test by LC-MS (SIM mode), AJS-ES, positive mode, SIM Ion: 483.2 [M+H]+
Column: YMC-Triart C18, 100×3.0 mm, particle size 1.9 μm, 12 nm
Mobile phase A: Dissolve 0.315 g of Ammonium formate in 1000 ml of water, add 0.5 mL of formic acid
Mobile phase B: Methanol
Sample solvent: Methanol+0.2% (v/v) Formic acid
Sample solution: approx. 100 mg of the sample in 20 mL
Flow: 0.6 mL/min
Injection volume: 3 μL
Autosampler cooled, 8° C.
Column temperature. 80° C.
Gradient:

| Min | Mobile phase A (%) | Mobile phase B (%) |
|---|---|---|
| 0 | 65 | 35 |
| 1 | 65 | 35 |
| 11 | 10 | 90 |
| 12 | 10 | 90 |
| 12.1 | 65 | 35 |
| 16 | 65 | 35 |

### Example 2: Enhanced Ames Test (EAT) for N-(3-(6-amino-5-(2(methyl)(nitroso)amino)ethoxy)pyrimidin-4-yl)-5-fluoro-2-methylphenyl)-4-cyclopropyl-2-fluorobenzamide

N-(3-(6-amino-5-(2(methyl)(nitroso)amino)ethoxy)pyrimidin-4-yl)-5-fluoro-2-methylphenyl)-4-cyclopropyl-2-fluorobenzamide was evaluated in the EAT performed under GLP following the EMA and FDA guidance (2023) for the EAT conditions for N-nitrosamines.

In this study N-(3-(6-amino-5-(2(methyl)(nitroso)amino)ethoxy)pyrimidin-4-yl)-5-fluoro-2-methylphenyl)-4-cyclopropyl-2-fluorobenzamide was evaluated for mutations in Salmonella typhimurium strains TA98, TA100, TA1535 and TA1537 and Escherichia coli strain WP2 uvrA pKM101, both in the absence and presence of 30% rat and hamster metabolic activation (β-naphthoflavone/phenobarbital-induced rat and hamster liver post-mitochondrial S-9 fraction). The test used pre-incubation method (30 minutes). N-(3-(6-amino-5-(2(methyl)(nitroso)amino)ethoxy)pyrimidin-4-yl)-5-fluoro-2-methylphenyl)-4-cyclopropyl-2-fluorobenzamide was formulated in dimethyl sulfoxide (DMSO) at concentrations of 5, 16, 50, 160, 500, 1600 and 5000 μg/plate.

Results of formulation analyses demonstrated stability and homogenicity (6 hrs at room temperature) of the N-(3-(6-amino-5-(2(methyl)(nitroso)amino)ethoxy)pyrimidin-4-yl)-5-fluoro-2-methylphenyl)-4-cyclopropyl-2-fluorobenzamide in the formulations and achieved concentrations within 100%+/−10% the nominal concentrations.

Precipitation was observed at 1600 μg/plate and/or 5000 μg/plate in all tester strains in the absence and presence of S-9.

Bacteriotoxicity was observed in strain TA1537 only at ≥1600 μg/plate in the presence of hamster S-9.

Following the recommendations of the EAT, in addition to the concurrent strain-specific positive controls, two nitrosamine positive controls, i.e. N-nitrosodiethylamine (NDEA) and N-methyl-N-nitroso-(2-phenylethyl)amine (MNPA) were used that are known to be mutagenic in the presence of S-9. In the presence of 30% rat S-9, NDEA induced mutations >2-fold in strain TA100. In the presence of hamster S-9 increases ≥2 or ≥3-fold were observed in all tester strains except strain TA1537. MNPA was mutagenic (≥2 or ≥3-fold increases) in Salmonella strains TA100, TA1535 and E. coli strain WP2 uvrA pKM101 in the presence of rat S-9 and hamster S-9. Increases up to 1.9 were observed in strain TA98 in the presence of hamster S-9. Following treatments with N-(3-(6-amino-5-(2(methyl)(nitroso)amino)ethoxy)pyrimidin-4-yl)-5-fluoro-2-methylphenyl)-4-cyclopropyl-2-fluorobenzamide, there were increases in revertant numbers that were ≥2-fold and ≥3-fold in strains TA100 and TA1535, respectively, in the presence of hamster S-9. N-(3-(6-amino-5-(2(methyl)(nitroso)amino)ethoxy)pyrimidin-4-yl)-5-fluoro-2-methylphenyl)-4-cyclopropyl-2-fluorobenzamide was not mutagenic without metabolic activation or in the presence of rat S-9.

In conclusion, N-(3-(6-amino-5-(2(methyl)(nitroso)amino)ethoxy)pyrimidin-4-yl)-5-fluoro-2-methylphenyl)-4-cyclopropyl-2-fluorobenzamide induced mutations under the conditions of the EAT in Salmonella strains TA100 and TA1535 in the presence of hamster S-9. It is therefore considered to be mutagenic under sensitive metabolic conditions.

### Example 3: Stability of Intermediate F8: Nitrosamine Content Over Time

In a 250 ML reactor under Argon was charged 7 g of F7 and 84 ML of iPrOAc. 5.12 g of HCl 37% was added to this suspension over 2 h. The reaction mixture was stirred overnight and quenched with 55 ML of distilled water. The biphasic suspension was warmed up to 30° C. 0.35 g of Charcoal was added, and the suspension was stirred for 2 h. The solids were filtered off and rinsed with 2 ML of distilled water. The clear aqueous layer was transferred to a clean reactor under Ar. The pH was adjusted to pH 6 with NaOH 30% (ca. 10 ML). 28.3 ML of EtOH was added and the suspension was warmed up to 60° C. At this temperature, the pH was slowly adjusted to pH 10 with NaOH 1N. The white suspension was cooled down to RT and filtered under protective atmosphere. The filter cake was rinsed with a mixture of ethanol/water and with ethanol. The F8 wet product was dried.

The content of nitrosamine in intermediate F8 was determined using method described in Example 1a.

| | Nitrosamine |
|---|---|
| F8 | in F8 (ppb) |
| Analyzed directly after weighting the sample | 86 |
| Analyzed after 52 h air exp. | 533 |

Further stability data have shown that the level of nitrosamine in the drug substance increases upon storage. The impact of protective gas (nitrogen) for storage vs storage in non-protected atmosphere, of light exposed vs dark conditions and the effect of temperature was investigated. In addition, the stability study samples pulled after 12 M of storage at different temperature and humidity were included into the assessment.

| | Nitrosamine |
|---|---|
| Storage condition (over 12 months)* | content in F8 (ppb) |
| T0 | 300 |
| 40° C., 75% (relative humidity) RH | 647 |
| 30° C., 65% RH | 595 |
| 5° C. | 323 |
| −20° C. | 276 |

*all samples were stored in double polyethylene-bags

The following observations were made:
While no increase of the nitrosamine-content was detected after storage for 12M in frozen (−20° C.) and only a minor rise was seen in cold (5° C.) conditions, a steep growth of >100% was observed upon storage at 30° C./65% RH and at 40° C./75% RH. The results were substantiated by additional stress testing.
When stored under inert gas, only a minor, yet constant formation of nitrosamine was seen over several days in the lab at room temperature whereas a rapid increase of the nitrosamine-level upon storage in an open vial was observed over the same period.
Exposure to light was shown to slightly accelerate conversion of F8 into nitrosamine.

The content of nitrosamine in intermediate F8 increased overtime upon exposure to the air or upon storage, e.g. by exposition to air, suggesting that handling of intermediate F8 should be done carefully.

### Example 4: Conversion of F8 to LOU064 Drug Substance with Low Content in Nitrosamine (i.e. N-(3-(6-amino-5-(2(methyl)(nitroso)amino)ethoxy)pyrimidin-4-yl)-5-fluoro-2-methylphenyl)-4-cyclopropyl-2-fluorobenzamide)

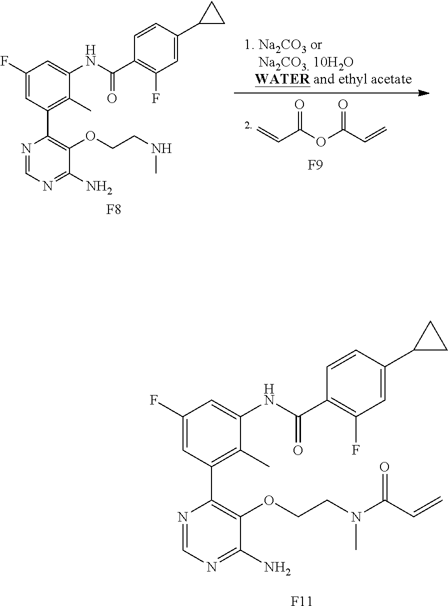

### Example 4a

Solution of sulfuric acid 0.5%: purified water (284.0 g) was charged into reaction vessel, sulfuric acid (1.4 g) was added, the reaction mixture was stirred for 30 min and set aside for later use.

Solution of acrylic anhydride: ethyl acetate (36.0 g) was charged into reaction vessel, acrylic anhydride (5.8 g, 1.05 equivalent) was added, and the mixture was stirred for 30 min and set aside for later use.

In a reactor under nitrogen, was charged (548.0 g) of ethyl acetate, purified water (100 g), 1.2 equiv of a base (e.g. low nitrite content sodium carbonate [Na₂CO₃·10H₂O: 5.6 g or anhydrous Na₂CO₃] and F8 (20.0 g). The reaction mixture was stirred and heated up to internal temperature IT=52-62° C.

The freshly prepared solution of acrylic anhydride was slowly added over 2 h. The dripping funnel was rinsed with ethyl acetate (11.4 g) and the reaction mixture was stirred for 30 min at internal temperature=52-62° C.

The phases were separated, and the aqueous layer was discarded. The organic layer (internal temperature=52˜62° C.) was washed with an aqueous solution of sulfuric acid 0.5% (285.4 g) as prepared above, then with purified water (200.0 g). Internal temperature IT was adjusted to 60˜66° C. A filtration was performed. The filter was rinsed with wet ethyl acetate (28.4 g and 1.5 g of purified water). The filtrates were combined, and the IT was adjusted to 58˜64° C. The organic phase was concentrated until the remaining material in the flask was 622.0 g/688 ml, in parallel, ethyl acetate 144.0 g was added dropwise. After concentration, a mixture of a crystal seed [0.060 g seed of anhydrous crystalline form A as disclosed in example 1 of WO2020/234779] in ethyl acetate (0.52 g) was charged into the flask and stirred for 15 min at IT=55˜61° C. After isotherm stirring, it was confirmed whether the reaction mixture in the flask is a suspension or not. If it is not a suspension, an additional mixture of LOU064 crystal seed 0.060 g of in ethyl acetate 0.52 g was added into the flask. If it is a suspension, the organic phase was concentrated until the remaining material in the flask was 622.0 g/688 ml at IT=55˜61° C., in parallel, ethyl acetate 72.0 g was added dropwise slowly. After distillation, the mixture was cooled to IT=22˜38° C., while the cooling time should be longer than 200 min. The organic phase was concentrated and controlled until the remaining material in the flask to be 622.0 g/688 ml at IT=22˜38° C. In parallel, ethyl acetate 556.0 g was added dropwise slowly. After concentration, the mixture was cooled down to −3˜3° C., while the cooling time should be more than 3 h. The mixture was stirred for 4 h, the cake was filtered, and the filter cake was rinsed with ethyl acetate (80.0 g) which was pre-cooled to −3˜3° C. in 2 portions. Remibrutinib was therefore obtained as a crystalline form (anhydrous modification A as disclosed in example 1 of WO2020/234779).

The content of nitrosamine in the drug substance was determined according to example 1b.

| | Nitrosamine | Water | Nitrosamine |
|---|---|---|---|
| | content | added | content in drug |
| Base | in F8 | (mole | substance |
| (1.2 mole equivalent) | (ppm)* | equivalent) | (F11) (ppm) |
| Na₂CO₃ anhydrous | 0.08 | 0 | 1.6 |
| nitrite content 1.3 ppm | | | |
| Example described in | | | |
| example 6 of | | | |
| PCT/IB2023/059664 | | | |
| Na₂CO₃ | 0.19 | 0 | 1.02 |
| Thermoscientific | | | |
| (A0447037) | | | |
| Nitrite content less than | | | |
| 0.25ppm (i.e. 0.06 ppm) | | | |
| Na₂CO₃—10H₂O | 0.19 | 0 | 1.62 |
| Thermoscientific | | | |
| (lot 10241212) | | | |
| Na₂CO₃—10H₂O | 0.19 | 12.5 | 0.64 |
| Thermoscientific | | | |
| (lot 10241212) | | | |
| Na₂CO₃—10H₂O | 0.08-0.15 | 12.5 | 0.32 |
| Thermoscientific | | | |
| (lot 10241212) | | | |
| Na₂CO₃—10H₂O | 0.08-0.15 | 25.2 | 0.21 |
| Thermoscientific | | | |
| (lot 10241212) | | | |
| Na₂CO₃—10H₂O | 0.08-0.15 | 37.8 | 0.25 |
| Thermoscientific | | | |
| (lot 10241212) | | | |
| Thermoscientific | 0.08-0.15 | 125 (no | 0.17 |
| (lot 10241212) | | work up) | |
| Na₂CO₃ anhydrous | 0.08 | 150 | 0.10 |
| Thermoscientific | | | |
| A0447037 | | | |

*Nitrosamine content in F8 was provided as a range because the nitrosamine impurity can be formed upon exposure to the air and/or under storage (Example 3)

### Example 4b: Larger Scale

Preparation of H₂SO₄ 0.5%: 66.6 g of sulfuric acid were diluted in 15 L of purified water at room temperature.

Preparation of acrylic anhydride solution (shortly before usage): 292 g of F9 were diluted in 1.8 kg of ethyl acetate.

Na₂CO₃ (281 g, 1.2 equivalent) was added to a suitable vessel and dissolved by addition of purified water (6 kg, 150 equiv compared to F8). Ethyl acetate (15 kg) was charged to the vessel and stirred 10 min at RT. F8 (1 kg—content of nitrosamine is 48 ppb) was charged into the vessel and the vessel was rinsed with ethyl acetate (13 kg). Total amount of ethyl acetate was 28 kg.

The reaction mixture was then warmed up to 60° C. As soon as the temperature was reached, the freshly prepared solution of F9 in ethyl acetate was added over 2 h. After the end of the addition the reaction mixture was stirred at 60° C. for 30 min. Once the mixture is mostly a solution (i.e. no longer a suspension), the reaction mixture was quenched with purified water (9 kg) and stirred for 10 min. The stirring was stopped, and the phases were allowed to separate over 30 min. The aqueous layer was discarded and the aqueous solution of H₂SO₄ 0.5% was added. The reaction mixture was stirred for 15 min. The stirring was stopped, and the phases were allowed to separate over 15 min. The aqueous layer was discarded, and purified water (10 kg) was added. The reaction mixture was stirred for 30 min at 60° C. The stirring was stopped, and the phases were allowed to separate over 30 min. The aqueous layer was discarded, and the organic phase was submitted to the same clear filtration/crystallization sequence as in example 4a. The obtained drug substance (F11) was isolated with a nitrosamine (N-(3-(6-amino-5-(2(methyl)(nitroso)amino)ethoxy)pyrimidin-4-yl)-5-fluoro-2-methylphenyl)-4-cyclopropyl-2-fluorobenzamide) content of 55 ppb as measured according to example 1b.

It can therefore be seen that the addition of water prior to the addition of acrylic anhydride has the effect of decreasing the amount of nitrosamine in the drug substance (F11). An addition of water prior to the addition of acrylic anhydride can reduce the amount of nitrosamine impurity. For example, addition of 12.5 mole equivalent of water prior to the addition of acrylic anhydride can reduced by a factor of 3 the amount of nitrosamine impurity. An additional factor 2 reduction can be achieved using 125 mole equivalent of water per mole of F8 prior to the addition of acrylic anhydride.

### Example 5: Synthesis of F8 with Various Degrees of Nitrosamine (i.e. N-(3-(6-amino-5-(2(methyl)(nitroso)amino)ethoxy)pyrimidin-4-yl)-5-fluoro-2-methylphenyl)-4-cyclopropyl-2-fluorobenzamide) Content

### Example 5a

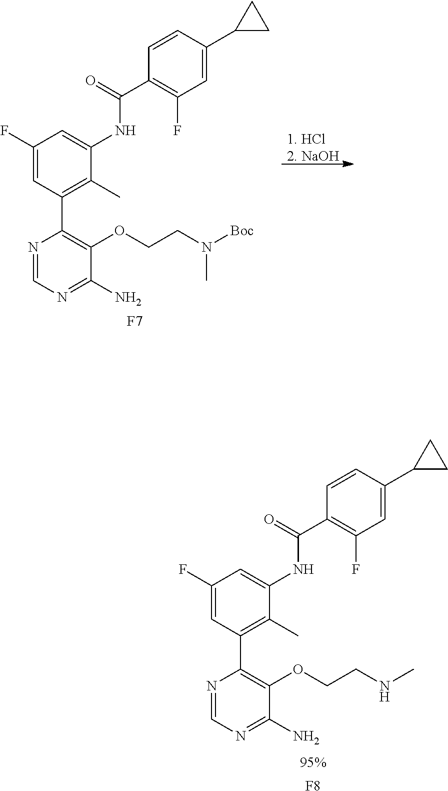

In a 250 ML reactor under Argon, was charged 7 g of F7 and 84 ML of iPrOAc. To this suspension was added over 2 h 5.12 g of HCl 37%. The reaction mixture was stirred overnight and quenched with 55 ML of tap water. The biphasic suspension was warmed up to 30° C. 0.35 g of Charcoal was added, and the suspension was stirred for 2 h. The solids were filtered off and rinsed with 2 ML of tap water. The clear aqueous layer was transferred to a clean reactor under Ar. The pH was adjusted to pH 6 with NaOH 30% (ca. 10 ML). 28.3 ML of EtOH was added and the suspension was warmed up to 60° C. At this temperature, the pH was slowly adjusted to pH 10 with NaOH 1N. The white suspension was cooled down to RT and filtered under protective atmosphere. The filter cake was rinsed with a mixture of ethanol water and with ethanol. The F8 wet product is dried. The content of nitrosamine (N-(3-(6-amino-5-(2(methyl)(nitroso)amino)ethoxy)pyrimidin-4-yl)-5-fluoro-2-methylphenyl)-4-cyclopropyl-2-fluorobenzamide) in F8 is 840 ppb.

### Example 5b: Optimized Condition for Decreasing Nitrosamine Content in F8

In a 250 ML reactor under Argon was charged 7 g of F7 and 84 ML of iPrOAc. To this suspension was added over 2 h 5.12 g of HCl 37%. The reaction mixture was stirred overnight and quenched with 55 ML of distilled water. The biphasic suspension was warmed up to 30° C. 0.35 g of Charcoal was added, and the suspension was stirred for 2 h. The solids were filtered off and rinsed with 2 ML of distilled water. The clear aqueous layer was transferred to a clean reactor under Ar. The pH was adjusted to pH 6 with a freshly open bottle of NaOH 30% (ca. 10 ML). A freshly open bottle of NaOH contributes to a low level of nitrite content (nitrite content can be determined using Griess test as disclosed in example 16). 28.3 ML of EtOH was added and the suspension was warmed up to 60° C. At this temperature, the pH was slowly adjusted to pH 10 with NaOH 1N. The white suspension was cooled down to RT and filtered under protective atmosphere. The filter cake was rinsed with a mixture of ethanol water and with ethanol. The F8 wet product is dried. The content of nitrosamine (N-(3-(6-amino-5-(2(methyl)(nitroso)amino)ethoxy)pyrimidin-4-yl)-5-fluoro-2-methylphenyl)-4-cyclopropyl-2-fluorobenzamide) in F8 is 31 ppb.

In conclusion, it can be seen that the use of purified (e.g. distilled water) as well as a low nitrite content NaOH (e.g. by using a freshly open bottle of NaOH) in the step of deprotection of F7 to give F8 has a significant impact on the nitrosamine content in F8.

| Exp. Name | Conditions | Nitrosamine (ppb) |
|---|---|---|
| Example 5a | No precaution taken, old bottle | 840 |
| | of NaOH 30%, tap water | |
| Example 5b | NaOH with freshly opened | 31 |
| | NaOH 30% solution and purified water | |

### Example 6: Conversion of F7 to F11 without Drying Intermediate F8

In a 250 ML reactor under Argon was charged 7 g of F7 and 84 ML of iPrOAc. To this suspension was added over 2 h 5.12 g of HCl 37%. The reaction mixture was stirred overnight and quenched with 55 ML of distilled water. The biphasic suspension was warmed up to 30° C. 0.35 g of Charcoal was added, and the suspension was stirred for 2 h. The solids were filtered off and rinsed with 2 ML of distilled water. The clear aqueous layer was transferred to a clean reactor under Ar. The pH was adjusted to pH 6 with a freshly open bottle of NaOH 30% (ca. 10 ML). 28.3 ML of EtOH was added and the suspension was warmed up to 60° C. At this temperature, the pH was slowly adjusted to pH 10 with NaOH 1N. The white suspension was cooled down to RT and filtered under protective atmosphere. The filter cake was rinsed with a mixture of ethanol water and with ethanol. The wet cake (nitrosamine content 7 ppb) was then directly engaged in the following reaction. To evaluate the F8 assay, a sample was dried and the weight loss on drying measured (LOD approx. 40%).

A 250 ML reactor under Argon was charged with 1.4 g of Na₂CO₃ and 30 g of purified water. 8.63 g of wet F8 (5.05 g calculated amount based on LOD) was suspended in 140 g of ethyl acetate and added to the reactor. The white suspension was heated up to 60° C. and a solution of 1.46 g of acrylic anhydride in 10 ML ethyl acetate was added over 2 h. The reaction mixture was stirred for an additional 30 min at 60° C. and 73 ML of purified water was added. After 10 min stirring at 60° C., the phases were separated, and the organic layer was washed with a H₂SO₄ 0.5% solution in purified water and with purified water. Azeotropic distillation of the ethyl acetate allowed for the crystallization of LOU064 which was isolated by filtration. The content of nitrosamine in the drug substance is 25 ppb.

### Examples 7-11: All Synthetic Steps for Making F7 as Disclosed in PCT/IB2023/059664

### Example 7—Preparation of F2

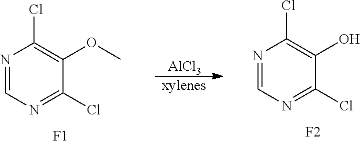

To a suspension of AlCl₃ in xylenes at 5° C. was added over 40 min a xylenes solution of F1. The mixture was warmed up to 30° C. over 60 min and stirred at this temperature overnight. EtOAc was added and the resulting solution was quenched over a 0.5 N aqueous solution of HCl at 0° C. for 1 h. The mixture was warmed to 25° C. and the phases were separated. The aqueous layer was discarded and the organic layer was concentrated. The resulting thin suspension was cooled to 20° C. at 0.3 K/min. The solids were filtered, the filter cake was washed with a 1:1 solution of xylenes and heptane and dried to afford F2 as a white solid in ca. 83% yield.

### Example 8—Preparation of F6

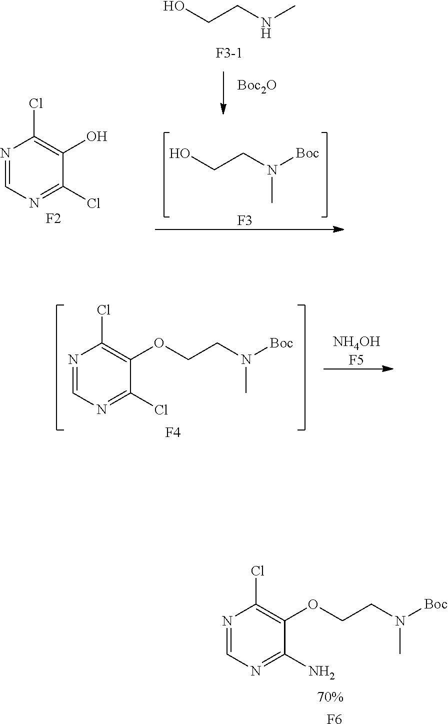

Preparation of the F3 solution: 13.0 g of water, 2.4 g of 30% sodium hydroxide solution, 68.0 g of toluene and 13.0 g of 2-methylaminoethanol were charged into reaction flask. Internal temperature was adjusted to 10-30° C. The reaction mixture was stirred for 25˜35 min. Boc anhydride (37.8 g, 1.00 equivalent) was added dropwise, and the reaction mixture was stirred at 10˜30° C. for another 6-12 hours. The reaction was quench with water (13.0 g) and the resulting biphasic mixture is stirred for 25-35 min. The lower water layer was removed, and the organic layer was washed with another portion of water (13.0 g). The organic layer was used directly for the next step.

Mistunobu reaction to F4: A solution of F3 (1.4 eq) in toluene was dried by Dean Stark distillation to reach a water content of NMT 0.07 wt %. To the dried solution of F3 at 20-30° C. were added triphenylphosphine (42 g, 1.32 eq) and the reaction mixture was stirred at room temperature until a clear solution was observed. The reactor was inertized and cooled to ca. −30° C. F2 (20 g, 1.0 eq) was then added followed by DIAD (31.8 g, 1.30 eq) was added over 4 to 8 h maintaining the internal temperature between −25° C. The slightly turbid solution was warmed up to 10° C. within 4 h and stirred for another 15 to 20 h between 5 and 15° C. After completion of the reaction, toluene was distilled at 55° C. yielding a slightly viscous brownish-yellow suspension. The mixture was cooled to 10° C. and n-heptane (140 g) is added. The mixture was stirred for 2 h affording a light brown, well stirrable suspension. The suspension was filtered, and the filter cake washed with cooled n-heptane. The filter cake containing triphenylphosphine oxide and H₂-DIAD was discarded. The combined mother and wash liquors were concentrated at JT 55° C. and 150 mbar to ca. ⅓ of their initial volume yielding a clear yellow solution of F4.

Amination to F6: The solvent of the solution of F4 was then switched to iPrOH via distillation and addition of iPrOH. To the yellow solution of F4 in iPrOH was added H₂O (3.5 w/w wrt F2) and 25 wt % NH₃ solution (3.5 w/w F2). The resulting yellow solution was stirred for 16 h at 70° C. A slight gas release (NH₃) was observed upon warming to 70° C. After completion of the reaction, the resulting yellow solution was cooled down to 45° C. over 40 min, and F6 seed crystals were added as a suspension in iPrOH. The suspension was aged for ca. 20 min. The thin suspension was then cooled to 10-20° C. at 10° C./h and aged for another 30 min. The suspension was filtered and the filter cake washed with a mixture (40 g) of H₂O and iPrOH (1:1). The wet product was dried at 50° C. under full vacuum (ca. 20 h) affording F6 as a white crystalline solid in ca. 70% yield.

### Example 9—Preparation of X6b

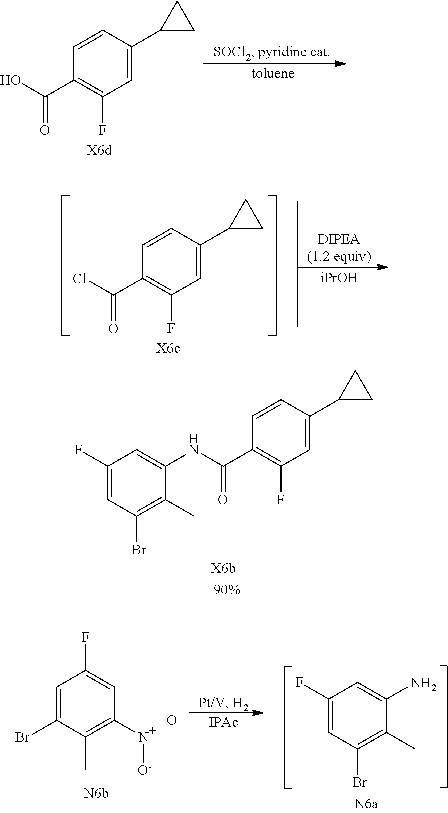

The synthesis of X6b was a highly convergent process that starts with the preparation of the N6a solution, the preparation of the acyl chloride X6c solution and the combination of the two solutions to form X6b.

Autoclave: preparation of the N6a solution: N6b (20 g, 1.0 eq) was charged in an autoclave under N₂ and diluted with isopropylacetate (105 g). Then, ca. 1 wt % of wet Pt(V)/C (0.126 g dry weight) was added and the atmosphere was changed from N2 to H₂. The hydrogenation was performed under 3 bars of H₂ for 12 h with an internal temperature below 30° C. At the end of the reaction, the suspension was filtered to remove the catalyst. The reactor and the filter cake were rinsed with isopropylacetate. The N6a solution can be azeotropically distilled to remove the water or used as it is.

Reactor A: preparation of X6c solution: Under N₂ atmosphere, X6d (17 g, 1.1 eq) was suspended in toluene (56 g). A catalytic amount of pyridine was added, and the reaction mixture was heated to 50° C. Thionyl chloride was then added dropwise over 2 h and the resulting mixture was stirred for 1 h at 50° C. The turbid solution was then distilled to half the volume, the reactor was refilled to its initial volume with toluene and the operation was repeated to remove the excess thionyl chloride. The X6c mixture was then cooled down to RT.

Reactor A: formation of X6b: To the solution of X6c (1.1 eq) in toluene was added over 1 h the formerly prepared solution of N6a (1.0 eq) in iPrOAc. At the end of the addition, DIPEA (13.4 g, 1.2 eq) was carefully added over 2 h. The reaction mixture was stirred for 3 h after the end of the DIPEA addition and the reaction was quenched with iPrOH (26.4 g). The reaction was stirred overnight at RT (room temperature) and the suspension was filtered. The wet cake was rinsed with iPrOH and iPrOH/water. The cake was discharged and dried under reduced pressure. X6b was typically isolated in 87-93% yield.

### Example 10a: Optimization of Suzuki Condition for Conversion of X6a into F7

Previously, it was reported (DOI: 10.1021/acs.jmedchem.9b01916) that the coupling reaction between F6 and X6a was carried out using 1 eq of F6, 1.15 eq of X6a, 5 mol % Pd(PPh₃)₂Cl₂, 3 eq of Na₂CO₃, 12 vol DME, 10 vol water at 75° C. for 8 h with a conversion of 74% isolated yield.

The cross-coupling reaction was optimized in order to replace the DME solvent with a class 3 solvent, suitable for commercial process, while also lowering the Pd loading and the production cost

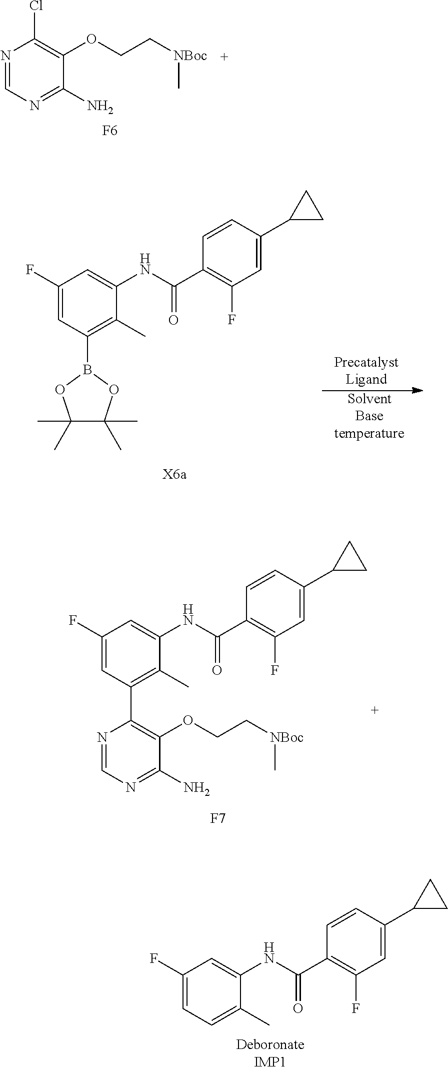

### Design and Experiment Details

1) Screened Suzuki 12 precatalysts and 6 solvent systems (80° C.: tert-Amyl alcohol, CPME and Toluene; 60° C.: THF, Me-THF and MeCN, combined with water respectively), using 1.15 eq. X6a at 2.0 mol % Pd level in the presence of 3.0 eq. K₃PO₄, after 16 h, found a series of precatalyst/solvent combinations that can promote reaction with full conversion, De-Boronate is the major side-product; decided to carry out full ligand screening in both Toluene (80° C.) and Me-THF (60° C.)
2) Screened 48 ligands in 10.0 vol. Me-THF/3.0 vol. water at 60° C. or in 10.0 vol. Toluene/3.0 vol. water at 80° C., using 2.0 mol % Pd(OAc)₂, 1.1 eq. Boronate and 3.0 eq. K₃PO₄, after 16 h, found 5 ligands (RuPhos, dppf, S-Phos, Cy₃P·HBF₄ and Ph₂P (t-Bu)) can promote reactions with full conversion with leading Prod/IS in Me-THF/water at 60° C., and De-Boronate side-product can be controlled at 3% to 8% level

| | | Temperature | | | De-Boronate/ |
|---|---|---|---|---|---|
| Solvent | Ligand | (° C.) | Base | Conversion | Prod |
| Toluene | Cy₃P—HBF₄ | 80 | K₃PO₄ | 97% | 17% |
| Me—THF | Cy₃P—HBF₄ | 60 | K₃PO₄ | 100% | 4% |
| Me—THF | RuPHOS | 60 | K₃PO₄ | 100% | 4% |
| Me—THF | S—PHOS | 60 | K₃PO₄ | 100% | 8% |
| Me—THF | Ph₂P(tBu) | 60 | K₃PO₄ | 100% | 3% |
| Me—THF | Cy—BIPHEP | 60 | K₃PO₄ | 99% | 8% |
| Me—THF | SPHOS—SO₃Na | 60 | K₃PO₄ | 99% | 6% |
| Me—THF | dppf | 60 | K₃PO₄ | 100% | 3% |

3) Keep P:Pd at 2:1 ratio, screened 6 Pd precursors (Pd(OAc)₂, [Pd(C₃H₅)Cl]₂, Pd(TFA)₂, Pd(MeCN)₂Cl₂, Pd₂ (dba)₃ and PdBr₂) at 1.0 mol % Pd level, combined with RuPhos, dppf, S-Phos, Cy₃P·HBF₄ and Ph₂P (t-Bu) respectively, in the presence of 3.0 eq. K₃PO₄ and 1.05 eq. X6a in 10.0 vol. Me-THF/3.0 vol. water at 60° C., after 16 h, still keep Cy₃P·HBF₄ and Ph₂P (t-Bu) as optimal ligand candidates, meanwhile, Pd(TFA)₂, Pd(MeCN)₂Cl₂ and PdBr₂ as leading Pd precursors

| | | | | | | De- |
|---|---|---|---|---|---|---|
| | | Temperature | | | | Boronate/ |
| Solvent | Ligand | (° C.) | Pd Precursor | Base | Conversion | Prod |
| Me—THF | Ph₂P(t-Bu) | 60 | Pd(MeCN)₂Cl₂ | K₃PO₄ | 100% | 2% |
| Me—THF | RuPhos | 60 | Pd(MeCN)₂Cl₂ | K₃PO₄ | 100% | 2% |
| Me—THF | Ph₂P(t-Bu) | 60 | Pd(TFA)₂ | K₃PO₄ | 100% | 1% |
| Me—THF | Ph₂P(t-Bu) | 60 | PdBr₂ | K₃PO₄ | 100% | 2% |
| Me—THF | Cy₃P•HBF₄ | 60 | Pd(TFA)₂ | K₃PO₄ | 100% | 3% |

4) Keep P:Pd at 2:1 ratio, using Cy₃P·HBF₄ and/or Ph₂P (t-Bu) as ligand, combined with Pd(TFA)₂, Pd(MeCN)₂Cl₂ and PdBr₂ respectively, screened Pd loading from 0.1 to 2.0 mol % in the presence of 3.0 eq. K₃PO₄ and 1.05 eq. X6a in 10.0 vol. Me-THF/3.0 vol. water at 60° C., after 16 h, found Pd(MeCN)₂Cl₂/Ph₂P (t-Bu) is the leading optimal precatalyst combination, and Pd loading can be dropped to 0.3 to 0.5 mol %, De-Boronate/Prod can be controlled at around 1%

| | | | | | | | De- |
|---|---|---|---|---|---|---|---|
| | | Temperature | | | Precatalyst | | Boronate/ |
| Solvent | Ligand | (° C.) | Pd Precursor | Base | Loading | Conversion | Prod |
| Me—THF | Ph₂P(t-Bu) | 60 | Pd(TFA)₂ | K₃PO₄ | 0.8 mol % | 100% | 2% |
| Me—THF | Ph₂P(t-Bu) | 60 | Pd(MeCN)₂Cl₂ | K₃PO₄ | 0.8 mol % | 100% | 2% |
| Me—THF | Ph₂P(t-Bu) | 60 | Pd(MeCN)₂Cl₂ | K₃PO₄ | 0.5 mol % | 100% | 1% |
| Me—THF | Ph₂P(t-Bu) | 60 | Pd(TFA)₂ | K₃PO₄ | 1.0 mol % | 100% | 2% |
| Me—THF | Ph₂P(t-Bu) | 60 | Pd(TFA)₂ | K₃PO₄ | 1.5 mol % | 100% | 2% |
| Me—THF | Ph₂P(t-Bu) | 60 | Pd(MeCN)₂Cl₂ | K₃PO₄ | 0.3 mol % | 99% | 1% |
| Me—THF | Ph₂P(t-Bu) | 60 | Pd(MeCN)₂Cl₂ | K₃PO₄ | 1.5 mol % | 100% | 2% |
| Me—THF | Ph₂P(t-Bu) | 60 | PdBr₂ | K₃PO₄ | 1.0 mol % | 100% | 2% |
| Me—THF | Ph₂P(t-Bu) | 60 | Pd(MeCN)₂Cl₂ | K₃PO₄ | 2.0 mol % | 100% | 2% |
| Me—THF | Ph₂P(t-Bu) | 60 | PdBr₂ | K₃PO₄ | 0.5 mol % | 100% | 1% |
| Me—THF | Cy₃P•HBF₄ | 60 | Pd(MeCN)₂Cl₂ | K₃PO₄ | 1.0 mol % | 99% | 3% |

5) Using Pd(MeCN)₂Cl₂/Ph₂P (t-Bu) as the optimal precatalyst combination and 1.05 eq. X6a, screened Pd loading from 0.1 to 0.5 mol % in the presence of K₂CO₃, Cs₂CO₃, K₃PO₄ and KF respectively, found K₃PO₄ is the optimal base, 0.3 to 0.5 mol % Pd(MeCN)₂Cl₂/Ph₂P (t-Bu) precatalyst is suggested in scaled-up reaction.

### Most Efficient Conditions

1) 1.0 eq. F6 1.05 eq. X6a, 0.5 mol % Pd(MeCN)₂Cl₂, 1.0 mol % Ph₂P (t-Bu), 3.0 eq. K₃PO₄ in 10.0 vol. Me-THF/3.0 vol. water at 60° C. for 16 h, reaction achieved a full conversion with 90.6% HPLC IPC purity, 1% De-Boronate/Prod.
2) 1.0 eq. F6, 1.05 eq. X6a, 0.3 mol % Pd(MeCN)₂Cl₂, 0.6 mol % Ph₂P (t-Bu), 3.0 eq. K₃PO₄ in 10.0 vol. Me-THF/3.0 vol. water at 60° C. for 16 h, reaction achieved 99% conversion with 88.5% HPLC IPC purity, 1% De-Boronate/Prod.

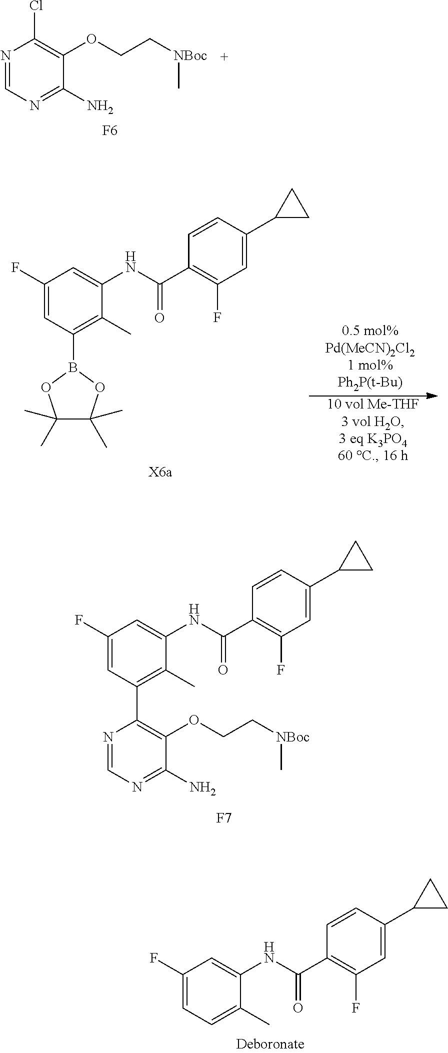

### Next Optimal Conditions

1) 1.0 eq. F6, 1.05 eq. X6a, 0.8 mol % Pd(TFA)₂, 1.6 mol % Ph₂P (t-Bu), 3.0 eq. K₃PO₄ in 10.0 vol. Me-THF/3.0 vol. water at 60° C. for 16 h, reaction achieved a full conversion with 90.9% HPLC IPC purity, 2% De-Boronate/Prod.
2) 1.0 eq. F6, 1.05 eq. X6a, 0.8 mol % Pd(MeCN)₂Cl₂, 1.6 mol % Ph₂P (t-Bu), 3.0 eq. K₃PO₄ in 10.0 vol. Me-THF/3.0 vol. water at 60° C. for 16 h, reaction achieved a full conversion with 91.2% HPLC IPC purity, 2% De-Boronate/Prod.

### Example 10b—Preparation of F7 One Pot-Borylation-Suzuki Cross Coupling from X6b Using Optimized Condition from Example 10a

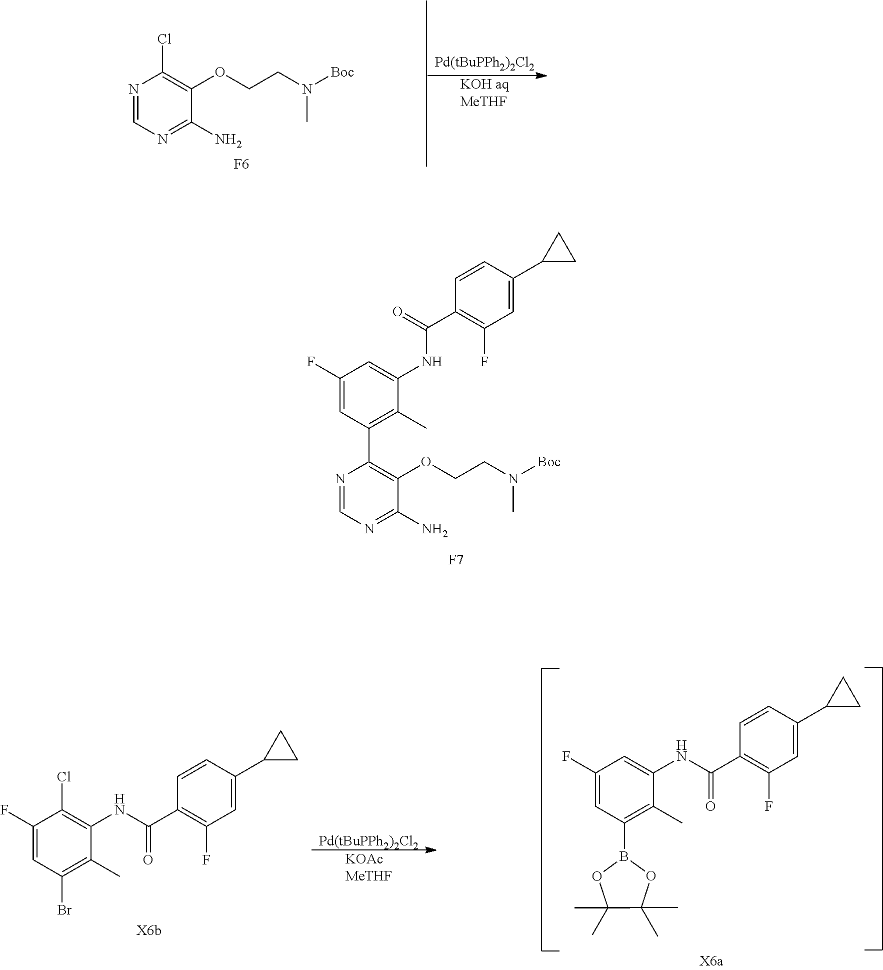

Miyaura borylation: X6b (1.0 eq), B₂pin₂ (1.06 equiv) and KOAc (2.5 equiv) were charged in a reactor under N₂ atmosphere containing degassed Me-THF. Water content of the reaction mixture was measured and adjusted between 1000 and 2500 ppm. After inertisation of the vessel, a solution of Pd(MeCN)₂Cl₂ (0.5 mol %) in degassed MeTHF and a solution of PPh₂tBu (1.0 mol %) in degassed MeTHF were successively added. The reaction mixture was then heated to 70° C. for 16 h.

Suzuki coupling: Once full conversion of X6b is achieved (X6b<0.25%, conversion is about 98%), the reaction mixture was cooled to RT and the reaction mixture was quenched with an aqueous solution of KOH (21% wt/wt). The aqueous layer was separated and discarded and a fresh portion of aqueous solution of KOH (21% wt/wt) was added. F6 (0.96 equiv compared to X6b) was added as a solid followed by, after appropriate degassing, a second portion of PPh₂tBu (2 mol %) in degassed MeTHF and a second portion of Pd(MeCN)₂Cl₂ (1 mol %) in degassed MeTHF. The reaction mixture was then heated to 60° C. for ca. 24 h. After completion of the reaction, an aqueous solution of N-acetyl cysteine was added to the reaction mixture at 60° C. After stirring for 2 h, the aqueous layer was discarded. Another portion of aqueous N-acetyl cysteine solution was added, and the pH was adjusted ≥9.5 by addition of aq. solution of KOH. After stirring for 2 h, the aqueous layer was discarded. The organic layer was then washed with water for 30 min and the aqueous layer was discarded. The solution was filtered at 60° C. over active charcoal and the solution was concentrated to half its volume by distillation under reduced pressure. n-Heptane was slowly added, and the resulting suspension was cooled to 20° C., stirred for 2 h and filtered. The filter cake was washed with a mixture of 1:5 Me-THF and n-heptane. In case the purity was not satisfactory, the wet cake can be re-slurried in Me-THF and n-heptane (1:5). The cake is discharged and dried under reduced pressure. F7 is typically isolated in 92% yield.

### Example 10c—Development of a One-Pot Borylation/Suzuki Cross-Coupling Using Tetrahydroxydiboron to Use in the Preparation of F7

A one-pot borylation/Suzuki cross-coupling using tetrahydroxydiboron was developed for the synthesis of F7 from X6b using BBA as borylating reagent. This process was characterized by the utilization of remarkably low loadings of Pd-catalyst, the avoidance of pinacol hydrate precipitates in the final product and the use of methanol as a green alcoholic solvent over both steps. This process addressed some of the previous problems associated with the use of bis(pinacolato)diboron as borylating reagent, thus becoming a more atom efficient and cost-effective approach. Results below demonstrated the feasibility of this one-pot process in a 2.2 g scale using a FlexyALR reactor.

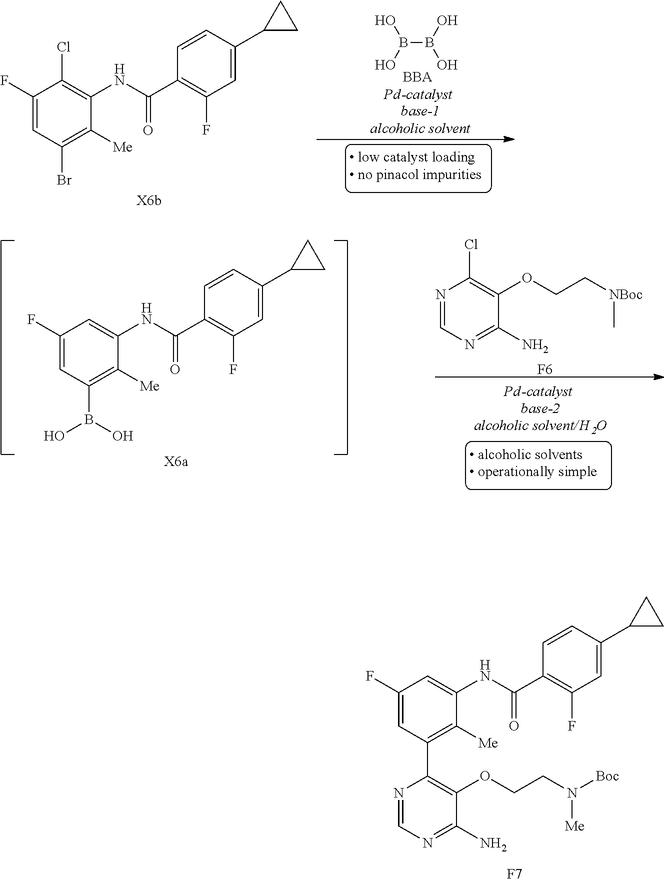

### Overview of Reactions

### Results and Discussion

Miyaura Borylation: In order to develop optimal reaction conditions for the Miyaura borylation of using BBA, crucial reaction parameters such as the catalytic system, base, solvent and temperature were screened. This borylation was restricted to the utilization of Pd(II)-precatalysts which promote fast Pd(0) formation. Indeed, the utilization of 2nd generation Buchwald precatalysts in combination with two equivalents of additional ligand, proved to be the most efficient catalytic system in our reaction (Table 1, entries 1-6). Out of all the screened precatalysts, only Pd-XPhos-2G afforded full conversion of the starting material while providing the highest yield and selectivity towards the formation of X6a (entry 2). In a similar manner, the utilization of ethylene glycol as additive also proved to be highly beneficial, as full conversion could not be achieved without it (entry 1 vs 2). BBA can be in situ stabilized through the formation of the corresponding boronic ester derivative, allowing the reduction in the amount of borylating reagent and Pd while increasing the rate of the borylation. A further reduction of the catalyst loading was attempted (entries 8-10). Surprisingly, reducing the catalyst loading afforded lower amounts of reduced and dimerized products IMP1 and IMP2, while still affording almost full conversion of X6b (entry 8). Additionally, higher conversion was observed by increasing the reaction time, thus suggesting that BBA was still present in the reaction mixture (entry 9). These results could indicate the formed boronic acid might undergo Pd(II)-catalyzed decomposition pathways, and that higher amount of Pd source in the presence of trace amount of oxygen might favor this pathway. Finally, simply by increasing the reaction temperature to 50° C., full conversion to the final product was observed in high selectivity and yield (entry 10).

TABLE 1
Screening results from the Miyaura borylation using BBA and KOAc.

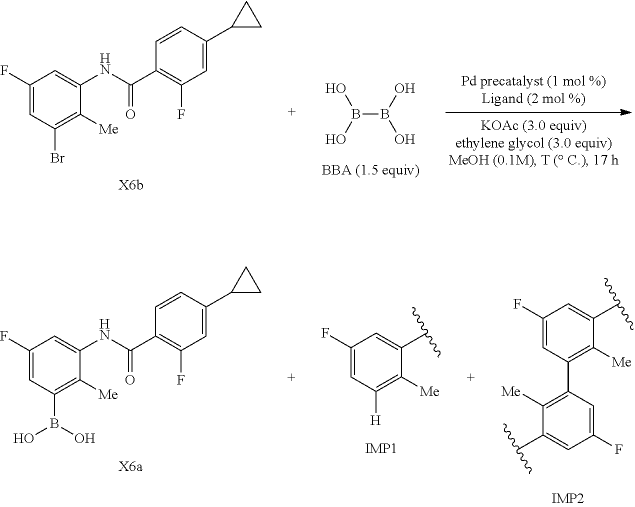 [SMILES: Cc1c(Br)cc(F)cc1NC(=O)c1ccc(C2CC2)cc1F ; OB(O)B(O)O ; Cc1c(NC(=O)c2ccc(C3CC3)cc2F)cc(F)cc1B(O)O ; *c1cc(F)ccc1C ; *c1cc(F)cc(-c2cc(F)cc(*)c2C)c1C]

| | | | | | IMP1 | IMP2 |
|---|---|---|---|---|---|---|
| Entryª | Pd-precatalyst | T (° C.) | X6b (%)^{a} | X6a (%)^{a} | (%)^{a} | (%)^{a} |
| 1 | XPhos-2G | 40 | 4 | 76 | 9 | 8 |
| 2 | XPhos-2G | 40 | 0 | 87 | 8 | 5 |
| 3 | Aphos-2G | 40 | 16 | 38 | 16 | 28 |
| 4 | RuPhos-2G | 40 | 43 | 39 | 11 | 4 |
| 5 | SPhos-2G | 40 | 50 | 36 | 7 | 3 |
| 6 | cataCXium-3G | 40 | 27 | 37 | 29 | 3 |
| 7 | Pd(PCy₃)₂ | 40 | 54 | 26 | 14 | 4 |
| 8 | XPhos-2G (0.25%) | 40 | 5.8 | 88.5 | 4.6 | 1.0 |
| 9 | XPhos-2G (0.25%)^{b} | 40 | 3.7 | 90.9 | 4.3 | 1.1 |
| 10 | XPhos-2G (0.25%) | 50 | 0 | 94.9 | 4.6 | 0.5 |

Reaction conditions: X6b (1.0 equiv), BBA (1.5 equiv), KOAc (3.0 equiv), ethylene glycol (3.0 equiv), Pd-precatalyst (1 mol %), Ligand (2 mol %), MeOH (0.1 M), T (C), 17 h. ^{a}Liquid Chromatography Area Percent of compound (LCAP). ^{b}Reaction time 20 h.

The reaction was also evaluated by replacing the ethylene glycol with an amine base, DIPEA, and other Buchwald precatalysts to determine if the results for the Miyaura Borylation could be further improved and the amount of working catalysts (Table 2, entries 1-5) increased. Although most catalysts did not perform as well under these conditions, an improvement was found by using Pd-cataCXium 3G (entry 5). Although slightly higher amounts of IMP1 and IMP2 were formed as compared to the previously optimized conditions, these results were promising considering cataCXium outperformed XPhos when used in combination with DIPEA (entry 5 vs 1). Further to this result, we screened additional critical reaction parameters to determine if this result could be further improved (entries 6-9). In consideration of our previous results, a reduction of the catalyst loading was first examined (entry 6). Importantly, we discovered that 0.05 mol % Pd was enough to drive the reaction to completion, suggesting that the catalytic activity of Pd-cataCxium-3G under these conditions was much higher than that of Pd-XPhos-2G. Importantly, heating to 50° C. was found optimal, as decreasing the temperature resulted in incomplete reactions (entry 7). Surprisingly, we discovered that the addition of ethylene glycol was detrimental for the conversion of the reaction, thus suggesting that cyclic diboron species might be less reactive under these conditions (entry 8). Although a remarkably high catalytic activity was seen under these newly optimized conditions, the relative amounts of byproducts IMP1 and IMP2 could not be further decreased, and the conditions based on the utilization of Pd-Xphos-2G, KOAc and ethylene glycol remained superior.

TABLE 2
Screening results from the Miyaura borylation using BBA and DIPEA.

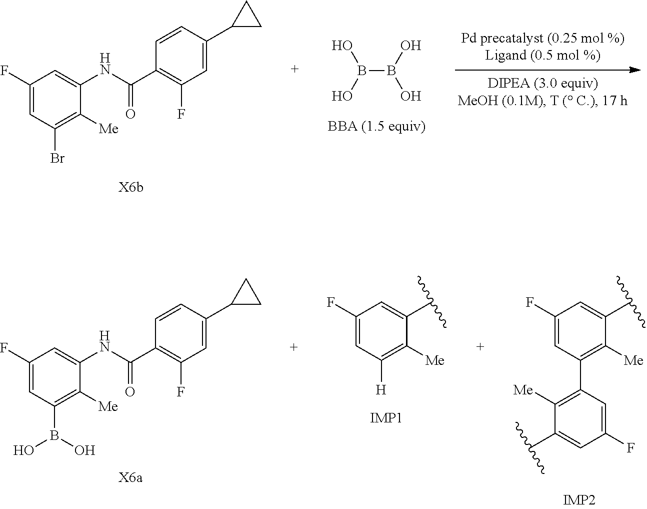 [SMILES: Cc1c(Br)cc(F)cc1NC(=O)c1ccc(C2CC2)cc1F ; OB(O)B(O)O ; Cc1c(NC(=O)c2ccc(C3CC3)cc2F)cc(F)cc1B(O)O ; *c1cc(F)ccc1C ; *c1cc(F)cc(-c2cc(F)cc(*)c2C)c1C]

| | | | | | IMP1 | |
|---|---|---|---|---|---|---|
| Entry | Pd-precatalyst | T (° C.) | X6b (%)^{a} | X6a (%)^{a} | (%)ª | IMP2 |
| 1 | Xphos-2G | 50 | 26 | 61 | 9 | 4 |
| 2 | Sphos-2G | 50 | 39 | 52 | 6 | 4 |
| 3 | PAphos-2G | 50 | 23 | 65 | 6 | 6 |
| 4 | PCy3-2G | 50 | 57 | 30 | 7 | 6 |
| 5 | cataCXium-3G | 50 | 0 | 87 | 7 | 6 |
| 6 | cataCXium-3G (0.05%) | 50 | 0 | 86 | 8 | 6 |
| 7 | cataCXium-3G (0.05%) | rt | 23 | 68 | 6 | 3 |
| 8 | cataCXium-3G b | rt | 69 | 20 | 11 | 1 |

Reaction conditions: X6b (1.0 equiv), BBA (1.5 equiv), DIPEA (3.0 equiv), Pd-precatalyst (0.25 mol %), Ligand (0.5 mol %), MeOH (0.1 M), T (C), 17 h. ^{a}Liquid Chromatography Area Percent of compound (LCAP). ^{b}Ethylene glycol (3.0 equiv) was added to the reaction mixture.

Suzuki Cross-Coupling: Having determined two sets of optimized conditions for the synthesis of boronic acid X6a by using BBA as borylating reagent, the viability of the subsequent Suzuki coupling was then studied with the ultimate goal of developing a one-pot process for the synthesis of F7. For this purpose, the Suzuki-coupling of X6a and F6 under Molanders previously developed (Gurung, S. R., et al., Org. Process Res. Dev. 2017, 21, 65-74) reaction conditions at 60° C. (Table 3, entry 1) was attempted. Disappointingly, uneven and incomplete conversion of X6a and F6 was observed after heating to 60° C. for 17 hours. Additionally, F6 was found to partially react through an SNAr pathway with EtOH to form the corresponding ether. At this point, it was considered whether the utilization of milder organic bases such as amines could help diminish this side reaction. Indeed, the utilization of Et₃N resulted in minimal formation of the C—O coupling product, leading to an even and almost complete conversion of X6a and F6 (entry 2). It was surprising to find that MeOH was superior to EtOH to provide full conversion of X6a and F6, and higher yields of the coupled product (entry 3). Additionally, F7 precipitated out directly from the reaction mixture, thus simplifying significantly the final workup purification. Although the formation of reduced and dimerized products IMP1 and IMP2 evidenced the presence of trace amount of oxygen in the reaction solvent, it was expected that the scale up the process should effectively eliminate this problem (entries 1-3).

TABLE 3
Screening results from the Suzuki coupling with F6.

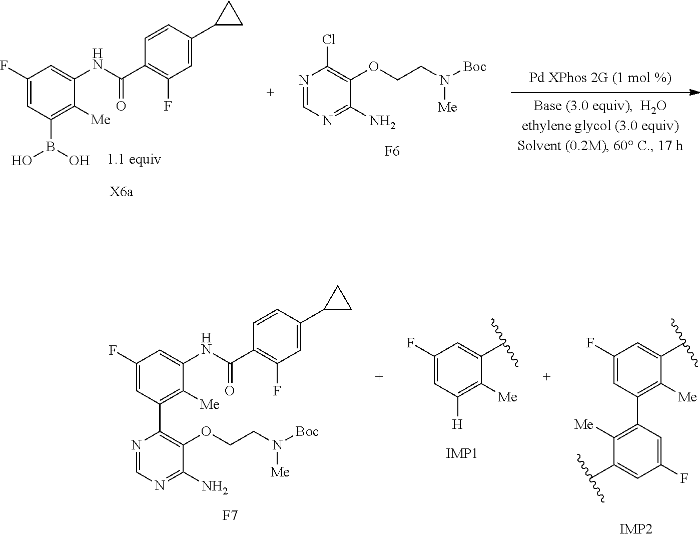

| Entry | Base | Solvent | X6a (%)^{a} | F6 (%)^{a} | F7 (%)^{a} | IMP1 (%)ª | IMP2 (%)^{a} |
|---|---|---|---|---|---|---|---|
| 1 | K₃PO₄ | EtOH | 43 | 15 | 28 | 8 | 5 |
| 2 | Et₃N | EtOH | 0 | 5 | 67 | 20 | 9 |
| 3 | Et₃N | MeOH | 0 | 0 | 77 | 7 | 13 |

Reaction conditions: X6a (1.1 equiv), F6 (1.0 equiv), Base (3.0 equiv), ethylene glycol (3.0 equiv), Pd-XPhos 2G (1.0 mol %), Solvent (0.2 M), 60° C., 17 h. ^{a}Liquid Chromatography Area Percent of compound (LCAP).

One-pot borylation and coupling: As Pd-XPhos 2G and Pd-cataCXium 3G were the leading precatalysts in the Miyaura borylation using BBA, it was decided to compare the efficiency of these two catalysts using optimized conditions in a one-pot process (Table 4). As shown in entry 1, Pd-XPhos 2G was demonstrated to be superior to Pd-cataCXium 3G in the one-pot procedure, affording a 79% isolated yield of F7 with a 78% purity starting from X6b. As expected, the workup and purification of F7 could performed through direct filtration and washing of the formed precipitate with a MeOH/H₂O mixture.

TABLE 4
Screening results from the one-pot reaction.

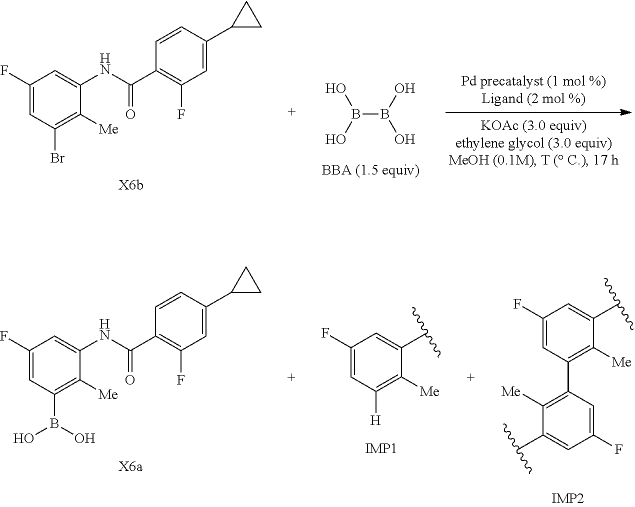 [SMILES: Cc1c(Br)cc(F)cc1NC(=O)c1ccc(C2CC2)cc1F ; OB(O)B(O)O ; Cc1c(NC(=O)c2ccc(C3CC3)cc2F)cc(F)cc1B(O)O ; *c1cc(F)ccc1C ; *c1cc(F)cc(-c2cc(F)cc(*)c2C)c1C]

| Entry | Pd-Precatalyst | X6a (%)^{a} | F6 (%)^{a} | F7 (%)^{a} | IMP1 (%)^{a} | IMP2 (%)^{a} |
|---|---|---|---|---|---|---|
| 1 | XPhos 2G | 5 | 1 | 74 (79%) | 8 | 8 |
| 2 | cataCXium 3G | 13 | 4 | 61 | 11 | 6 |

Miyaura borylation: Reaction conditions: X6b (1.0 equiv), BBA (1.5 equiv), KOAc (3.0 equiv), ethylene glycol (3.0 equiv), Pd-XPhos 2G (0.25 mol %), XPhos (0.5 mol %), MeOH (0.1 M), 50° C., 17 h. Suzuki-coupling: X6a (1.0 equiv), F6 (0.95 equiv), Et₃N (3.0 equiv), Pd-XPhos 2G (1.0 mol %), MeOH (0.1 M), 50° C., 17 h. ^{a}Liquid Chromatography Area Percent of compound (LCAP).

Scale-up: With conditions developed for both steps in MeOH, using the same precatalyst under mild conditions, the one-pot reaction was attempted on a larger scale (2.2 g of X6b) using a Flexy ALR-1 300 ml reactor (Table 5).

X6b (2.20 g, 1.0 equiv.), potassium acetate (1.76 g, 3.0 equiv.), ethylene glycol (1.0 ml, 3.0 equiv.) and MeOH (100 ml) were charged in a 300 ml FlexyALR reactor. The reaction mixture was degassed through successive vacuum/N2 cycles and a solid mixture of BBA (807 mg, 1.5 equiv.), Pd XPhos 2G (12 mg, 0.25 mol %) and XPhos (14 mg, 0.50 mol %) was added under N₂. After degassing a second time, the reaction was heated to 50° C. and stirred overnight. The mixture containing the boronic acid was then cooled to 20° C., and F6 (1.73 g, 0.95 equiv.), Pd XPhos 2G (24 mg, 0.5 mol %), Et₃N (2.5 ml) and degassed water (30 ml) were added under N₂. The reaction was degassed a third time and stirred at 60° C. overnight. Subsequently, it was cooled to 40° C. and concentrated under reduced pressure (ca. 40 ml MeOH removed). The reaction mixture was then cooled to 20° C. and stirred for 3 hours. The light brown suspension was filtered off, washed with a cold solution of MeOH/H₂O 4/1 (40 ml) and dried to afford F7 (1.87 g, 56%) as a brown solid.

TABLE 5
Scale up of the one-pot process.

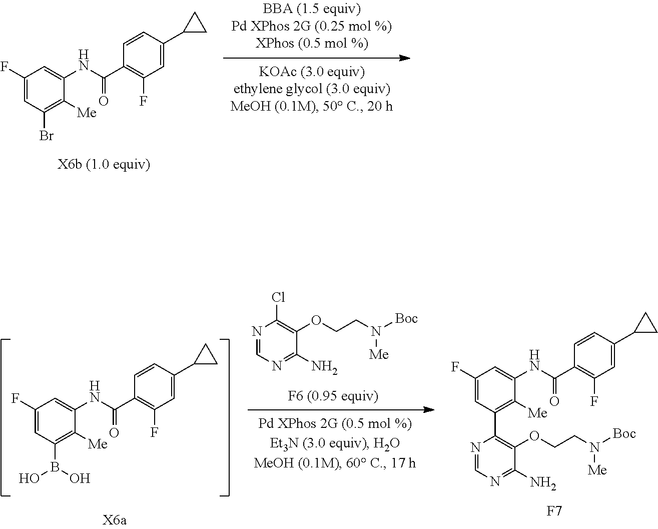

| Step | X6b (%)^{a} | X6a (%)^{a} | IMP1 (%)^{a} | IMP2 (%)^{a} | F6 (%)^{a} | F7 (%)^{a} |
|---|---|---|---|---|---|---|
| Borylation | 0 | 93.9 | 4.6 | 1.4 | | |
| Suzuki | | 21 | 7 | 1 | 3 | 63 (56) |

Miyaura borylation: Reaction conditions: X6b (1.0 equiv), BBA (1.5 equiv), KOAc (3.0 equiv), ethylene glycol (3.0 equiv), Pd-XPhos 2G (0.25 mol %), XPhos (0.5 mol %), MeOH (0.1 M), 50° C., 17 h. Suzuki-coupling: X6a (1.0 equiv), F6 (0.95 equiv), Et₃N (3.0 equiv), Pd-XPhos 2G (0.5 mol %), MeOH (0.1 M), 60° C., 17 h. ªLiquid Chromatography Area Percent of compound (LCAP).

The Miyaura borylation of X6b resulted in the generation of the desired intermediate X6a in an excellent yield and selectivity. Interestingly, as described by Molander for the utilization of Pd-XPhos 2G, the end of the borylation was evidenced through the sudden color change of the reaction mixture from white to light orange. Subsequently, the Suzuki-coupling was carried out adding F6, a new batch of catalyst, Et₃N and H₂O to the reaction mixture. Filtration and washing of the final product afforded F7 in a 56% isolated yield over both steps with an 87% IPC purity. Importantly, as we had anticipated, excluding all traces of oxygen by carrying out both steps in a reactor minimized the formation of byproduct IMP1 and IMP2.

### Example 12: Stability of LOU064 Drug Substance

In order to assess the stability behavior of the nitrosamine impurity (N-(3-(6-amino-5-(2(methyl)(nitroso)amino)ethoxy)pyrimidin-4-yl)-5-fluoro-2-methylphenyl)-4-cyclopropyl-2-fluorobenzamide) in LOU064 drug substance, an accelerated predictive stability (APS) was performed.

### Description of the Method:

A statistical based approach, Accelerated Predictive Stability (APS), using the humidity modified Arrhenius equation [Waterman and al., International Journal of Pharmaceutics 293 (1-2), 101-125 (2005)] is applied to understand stability behavior and predict the retest period. The design of the predictive study is based on the studies reported in the literature that demonstrates the modeling of observed degradation of solid oral dosage forms (Waterman et al., Pharmaceutical Research 24 (4), 780-790 (2007) and Waterman et al., Journal of Pharmaceutical Sciences, 99 (11), 4437-4452 (2010)). Short studies are conducted on open dish samples of representative batch(es) of drug substance at accelerated conditions using a wide range of temperatures and humidity with the goal of reaching the specification limit for the identified retest period limiting attributes at each condition as detailed below. Humidity determines water activity in the drug substance and can have a significant effect on reaction rates in solid drug substances; even for reactions, which themselves do not involve water. The humidity corrected Arrhenius equation reflects both the influence of the temperature, and the moisture on the kinetics of the degradation product formation. The resulting open-dish data are fitted to a humidity-corrected Arrhenius equation¹ using ASAPprime® (current version 6.0):

ln k=lnA−Ea/RT+B(% RH) Equation¹

### Humidity Corrected Arrhenius Equation [1];

where k is the degradation rate, A is the Arrhenius collision frequency, Eₐ is the activation energy for the chemical reaction, R is the gas constant, T is the temperature in Kelvin and B is a humidity sensitivity constant and percent relative humidity is indicated as % RH.

### Experimental Conditions:

3 batches of LOU064 drug substance were placed in open dish under the protocol summarized below:

TABLE 6
Experimental conditions APS

| | RELATIVE | | | | | |
|---|---|---|---|---|---|---|
| TEMPERATURE | HUMIDITY | STORAGE TIME IN DAYS | | | | |
| [° C.] | [% RH] | Initial | 7 | 14 | 21 | 28 |
| NA | NA | x | — | — | — | — |
| 50 | 75 | — | — | x | x | x |
| 60 | 11 | — | — | — | x | x |
| 60 | 50 | — | — | x | — | x |
| 60 | 75 | — | — | x | x | x |
| 70 | 31 | — | — | x | — | x |
| 70 | 50 | — | — | — | x | x |
| 70 | 75 | — | — | x | x | — |
| 80 | 50 | — | x | — | x | — |

x = analyzed (3 data points)

Samples of batches of LOU064 drug substance were prepared in duplicated in closed container, with one sample being purged with nitrogen and the other not.

TABLE 7
Experimental conditions APS closed vessel
with or without nitrogen purge

| | STORAGE TIME IN DAYS | | |
|---|---|---|---|
| TEMPERATURE [° C.] | Initial | 21 | 27 |
| NA | x | — | — |
| 60 | — | x | x |
| 70 | — | x | x |
| 80 | — | x | x |

x = analyzed as a single determination

The pulled samples were analyzed by HPLC-MS as per method described in example 1 and reported below in tables 8 and 9.

TABLE 8
APS behavior of the nitrosamine impurity in
LOU064 drug substance under open dish storage
at various temperature humidity conditions

| Temp. [° C.] | RH[%] | Time [days] | Nitrosamine impurity [ppb] |
|---|---|---|---|
| N/A | N/A | 0 | 99.1 |
| 80 | 51 | 7 | 105.3 |
| 50 | 75 | 14 | 74.9 |
| 60 | 50 | 14 | 77.0 |
| 60 | 75 | 14 | 69.8 |
| 70 | 31 | 14 | 77.9 |
| 70 | 75 | 14 | 82.6 |
| 50 | 75 | 21 | 75.8 |
| 60 | 11 | 21 | 87.1 |
| 60 | 75 | 21 | 82.7 |
| 70 | 50 | 21 | 83.2 |
| 70 | 75 | 21 | 83.2 |
| 80 | 51 | 21 | 75.5 |
| 50 | 75 | 28 | 106.5 |
| 50 | 75 | 28 | 106.8 |
| 60 | 11 | 28 | 113.5 |
| 60 | 50 | 28 | 106.6 |
| 60 | 75 | 28 | 119.1 |
| 70 | 31 | 28 | 113.3 |
| 70 | 50 | 28 | 108.3 |

The nitrosamine was found to be stable, neither increasing or decreasing beyond expected analytical variability under all APS open dish conditions.

TABLE 9
APS behavior of nitrosamine impurity in LOU064 drug substance with
and without nitrogen purge at various temperature conditions

| | | Nitrosamine[ppb] | |
|---|---|---|---|
| Temp. | Time | without | nitrosamine [ppb] |
| [° C.] | [days] | Nitrogen purge | with Nitrogen purge |
| N/A | 0 | 99.1 | 99.1 |
| 60 | 21 | 72.5 | 86.5 |
| 60 | 27 | 109.8 | 110.4 |
| 70 | 21 | 67.0 | 76.6 |
| 70 | 27 | 116.0 | 109.4 |
| 80 | 21 | 59.6 | 54.8 |
| 80 | 27 | 114.7 | 119.2 |

Comparing LOU064 drug substance stored closed under nitrogen versus closed without nitrogen, there is no difference observed beyond analytical variability.

### Conclusion

Based on the APS data, it is not expected that the nitrosamine impurity increases in the LOU064 drug substance, manufactured with low level of nitrosamine, as described in this invention. Nitrogen purge did not have any appreciable effect on nitrosamine formation.

### Example 13: Evaluation of the Drug Product Manufacturing Process on Increase of Nitrosamine Impurity (N-(3-(6-amino-5-(2(methyl)(nitroso)amino)ethoxy)pyrimidin-4-yl)-5-fluoro-2-methylphenyl)-4-cyclopropyl-2-fluorobenzamide)

The drug product (film coated tablet) was prepared as disclosed in WO2022/162513, example 8 using LOU064 drug substance substantially free of nitrosamine. The manufacturing process comprises steps of wet media milling, spray granulation and granule formation, final blend preparation and tableting.

The level of nitrosamine impurity (N-(3-(6-amino-5-(2(methyl)(nitroso)amino)ethoxy)pyrimidin-4-yl)-5-fluoro-2-methylphenyl)-4-cyclopropyl-2-fluorobenzamide) was measured at each step of the process and summarized in the table 10 below:

TABLE 10
content of nitrosamine variation during drug manufacturing process

| | | | | |
|---|---|---|---|---|
| | After wet- | | | |
| Drug substance | media milling | Granules | Final blend | Tablets |

Nitrosamine level in ppb

| | | | | |
|---|---|---|---|---|
| Less than 200 | 136.2 | 149.6 | 145.4 | 145.0 |

Conclusion: drug substance process (impurity content) is the main contributor for the presence of nitrosamine impurity in the drug product. Nitrosamine impurity levels are minimally affected by the drug product manufacturing process as disclosed in WO2022/162513, especially when the nitrosamine level in the drug substance is less than 200 ppb.

### Example 14: Stability of LOU064 Drug Product (Film Coated Tablets)

A statistical based approach, accelerated predictive Stability (APS), using the humidity modified Arrhenius equation as described in example 12, was used to understand stability behavior and predict the shelf life.

Several batches of LOU064 drug product (film coated tablets) prepared according to example 8 of WO2022/162513 with a drug substance substantially free of nitrosamine were analyzed for stability under different storage conditions. The content of nitrosamine (i.e. N-(3-(6-amino-5-(2(methyl)(nitroso)amino)ethoxy)pyrimidin-4-yl)-5-fluoro-2-methylphenyl)-4-cyclopropyl-2-fluorobenzamide) was analyzed by HPLC-MS as described in example 1.

TABLE 11
APS behavior of drug product LOU064 under open dish
storage at various temperature humidity conditions:

| | | | |
|---|---|---|---|
| Temp. (° C.) | RH(%) | Time (days) | nitrosamine [ppm] |

Requirements ≤2.0

| | | | |
|---|---|---|---|
| NA | NA | 0 | 0.37 |
| 80 | 50 | 7 | 0.38 |
| 50 | 75 | 14 | 0.35 |
| 60 | 50 | 14 | 0.38 |
| 60 | 75 | 14 | 0.36 |
| 70 | 31 | 14 | 0.37 |
| 70 | 75 | 14 | 0.32 |
| 50 | 75 | 21¹ | 0.35 |
| 60 | 11 | 21 | 0.36 |
| 60 | 75 | 21 | 0.35 |
| 70 | 50 | 21 | 0.32 |
| 70 | 75 | 21 | 0.33 |
| 80 | 50 | 21 | 0.34 |
| 50 | 75 | 28 | 0.33 |
| 50 | 75 | 28 | NT |
| 60 | 11 | 28 | 0.35 |
| 60 | 50 | 28 | 0.35 |
| 60 | 75 | 28 | 0.35 |
| 60 | 75 | 28 | NT |
| 70 | 31 | 28 | 0.36 |
| 70 | 50 | 28 | 0.34 |
| 70 | 75 | 28 | <0.32 |

At day 28, batches were further analyzed by XRPD.

TABLE 12
results identity by XRPD

| Condition | | | Identity by XRPD |
|---|---|---|---|
| T (° C.) | RH(%) | Time (days) | LOU064 DP |
| NA | NA | Initial | LOU064 detected, |
| | | | corresponds to |
| | | | modification A |
| 40 | 75 | 28 | LOU064 detected, |
| | | | corresponds to |
| | | | modification A* |
| 50 | 75 | 28 | LOU064 detected, |
| | | | corresponds to |
| | | | modification A* |
| 60 | 31 | 28 | LOU064 detected, |
| | | | corresponds to |
| | | | modification A* |
| 60 | 50 | 28 | LOU064 detected, |
| | | | corresponds to |
| | | | modification A* |
| 60 | 75 | 28 | LOU064 detected, |
| | | | corresponds to |
| | | | modification A* |

NA = not applicable
*Extra peaks which can be assigned to degradation of sodium stearyl fumarate Modification A as disclosed in Example 1 of WO2020/234779

Conclusion: No trend was observed for the nitrosamine impurity (i.e. the level of N-(3-(6-amino-5-(2(methyl)(nitroso)amino)ethoxy)pyrimidin-4-yl)-5-fluoro-2-methylphenyl)-4-cyclopropyl-2-fluorobenzamide did not increase) on the APS study even in the most extreme conditions. Furthermore, no changes were observed in term of polymorphic forms.

### Example 15: Method for Validating the Content of Nitrosamine in the Drug Product (e.g. Film Coated Tablet)

Method is also applicable for validating the content of nitrosamine in the drug substance In the description, the remibrutinib nitrosamine impurity (i.e. N-(3-(6-amino-5-(2(methyl)(nitroso)amino)ethoxy)pyrimidin-4-yl)-5-fluoro-2-methylphenyl)-4-cyclopropyl-2-fluorobenzamide) will be referred to as RNI.

RNI-d7 used below as reference substance has the following structure

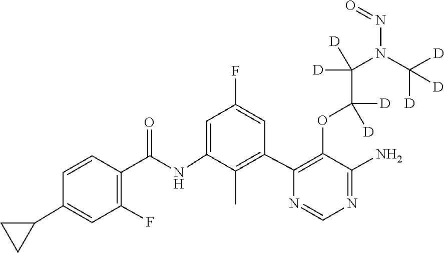 [SMILES: [2H]C([2H])([2H])N(N=O)C([2H])([2H])C([2H])([2H])Oc1c(N)ncnc1-c1cc(F)cc(NC(=O)c2ccc(C3CC3)cc2F)c1C]

HPLC-MS

| Principle | (U)HPLC with HRAM (High Resolution Accurate Mass) mass | | |
|---|---|---|---|
| | spectrometric detection and internal standard method | | |
| Reagents | | | |
| Methanol | HPLC gradient grade or equivalent, e.g. J. T. Baker 8402 | | |
| Ammonia 25% (w/V) | LC-MS grade or equivalent, e.g. Merck 533003 | | |
| Water | Purified | | |
| Reference substances | RNI | | |
| | RNI-d7) | | |
| Equipment | | | |
| Apparatus | (U)HPLC coupled to HRAM-MS (e.g. Thermo Orbitrap Exploris | | |
| | 120, or equivalent) | | |
| Column | Waters UPLC BEH C18 Length 100 mm, internal diameter 2.1 mm | | |
| | and particle size 1.7 μm, or equivalent column | | |
| Filters | 0.2 μm PVDF filters, or equivalent | | |
| Solvent | Water:MeOH = 20:80 (V/V) | | |
| Chromatographic | | | |
| conditions | | | |
| Mobile phase A | 0.025% (w/V) ammonia in water | | |
| | e.g. Add 1000 μL of 25% ammonia to 1000 ml of water and mix | | |
| | well | | |
| Mobile phase B | MeOH | | |
| | | Mobile phase | Mobile phase |
| | Time [min] | A [%] | B [%] |
| Gradient program (linear) | 0.0 | 60 | 40 |
| | 0.5 | 60 | 40 |
| | 8.0 | 40 | 60 |
| | 8.1 | 5 | 95 |
| | 9.0 | 5 | 95 |
| | 9.1 | 60 | 40 |
| | 12.0 | 60 | 40 |
| | Note: Depending on the LC instrument used, the final | | |
| | conditioning time can be extended in order to achieve | | |
| | satisfactory reconditioning of the chromatographic column. | | |
| Flow rate | 0.4 mL/min | | |
| Detection | High Resolution Mass Spectrometry Detector | | |
| Column temperature | 50° C. | | |
| Post column cooler | 40° C. | | |
| Auto-sampler temperature | 22.5° C. | | |
| Injection volume | 5 μL | | |
| Approximate retention times of | About 7 min (for information only) | | |
| RNI, RNI-d7 | | | |

| | |
|---|---|
| Note | A switch of the LC flow to the waste before and after the |
| | elution of RNI and RNI-d7 peak is recommended. |

| MS detector settings | |
|---|---|
| Ionization | H-ESI, Positive ions |
| Spray voltage | 3500 V |
| Sheath gas | 50 AU |
| Auxiliary gas | 5 AU |
| Sweep gas | 0 AU |
| lon transfer Tube Temp. | 300 ° C. |
| Vaporizer Temp. | 350 ° C. |
| Scan type | tMS² |
| Precursor ion (m/z) RNI | 483.1951 [M + H]⁺ |
| Precursor ion (m/z) RNI-d7 | 490.2390 [M + H]⁺ |
| RNI: Product ion (m/z) | 87.0553 ± 10 ppm (Quant) |
| | 397.1476 ± 10 ppm (Conf) |
| RNI-d7: Product ion (m/z) | (93.0929 ± 94.0992) ± 10 ppm (Quant) |
| | 398.1539 ± 10 ppm (Conf) |
| Resolution | 60000 |
| Isolation window (m/z) | 0.4 |
| HCD Collision energy (V) | 18 |
| RF Lens (%) | 70 |
| Maximum Injection Time (ms) | Auto |
| Expected LC Peak width (s) | 20 |

Note: All MS detector settings reported above are indicative and may be adapted and optimized for
different MS detectors as long as SST requirements are met, and MS conditions can be adjusted to
optimize the detection.

| | |
|---|---|
| System suitability test (SST) | |
| Blank | No peaks interfering with RNI and RNI-d7 peaks are |
| | present. |
| Repeatability (STD) | Sᵣₑₗ ≤ 20% (n = 6) (peak area of RNI in the standard |
| | solution) |
| Reporting limit | A signal-to-noise ratio of ≥10 for the peak of RNI in LOQ |
| | solution |
| | Note: S/N may not be possible to calculate if there is no |
| | noise in blank. In this case, SST is compliant. |
| Proportionality Factor (PF) | Proportionality of RNI to IS peak ratios between standard |
| | solution and LOQ solution: 0.7 ≤ PF ≤ 1.3 (start of |
| | sequence) |
| Drift of Standard solution | Drift less than or equal to ±25% |
| Note | Calculations in accordance with Ph. Eur. 2.2.46 |
| | “Chromatographic Separation Techniques” or USP <621>, |
| | “Chromatography, System Suitability Test”. |
| Procedure | |
| Internal standard solution (IS) | Dissolve RNI-d7 in methanol and dilute with solvent |
| (c RNI-d7 ≈ 5 ng/ml) | (approximately 5 ng RNI-d7/mL) |
| | E.g. Accurately weigh about 2.5 mg of RNI-d7 reference |
| | substance to 25 mL volumetric flask, dissolve in methanol |
| | and dilute with methanol to volume (c RNI-d7 ≈ 100 μg/mL). |
| | Further dilute 100 uL of the above solution to 10 mL with |
| | solvent (c RNI-d7 ≈ 1 μg/mL). |
| | Further dilute 1000 μL of the above solution to 200 mL with |
| | solvent. |
| Test solution | Approximately 2.5 mg LOU064/mL in IS (Approximately |
| | 2.5 mg LOU064/mL and approximately 5 ng RNI-d7/mL) |
| | E.g. Grind and homogenize 10 tablets (e.g. IKA mill can be |
| | used). Accurately weigh about 136 mg of tablet powder |
| | (equivalent to about 25 mg of LOU064) into a 10 ml |
| | volumetric flask. Add approximately 2/3 of flask volume of |
| | IS and shake on a mechanical shaker for at least 15 |
| | minutes. Fill the flask with IS to volume and shake well. |
| | Filter through 0.2 μm PVDF filter (or other suitable filter) into |
| | a HPLC vial, discard the first 0.5 mL |
| | Note: alternative test solution preparation may be used if |
| | validated. |
| RNI stock solution (stock SS) | Approximately 100 μg RNI/mL in methanol |
| (c RNI ≈ 100 μg/mL) | E.g. Weigh accurately about 5 mg of RNI reference |
| | substance into a 50 mL volumetric flask, dissolve in and |
| | dilute to volume with methanol. |
| Reference standard | 1 μg RNI/mL in IS (1 μg RNI /mL and approximately 5 ng of |
| Solution (REF) | RNI-d7/mL |
| (c RNI = 1 μg/mL) | E.g. pipette exact volume (500 μL) of Stock SS, taking into account |
| (c RNI-d7 ≈ 5 ng/ml) | exact weight and purity of RNI reference substance into 50 mL |
| | volumetric flask, according to the equation below. Dilute to volume |
| | with IS. |
| | $V = \frac{5 \times 100}{m \times P} \times 500$ |
| | where: |
| | V: Volume to be diluted (μL) |
| | m: mass of RNI reference standard (mg) |
| | P: purity of RNI reference standard (%) |
| Standard solution (STD) | 5 ng RNI/mL in IS (5 ng RNI/mL and approximately 5 ng RNI- |
| c RNI = 5 ng/ml | d7/mL) |
| (equivalent to 2000 ppb), | E.g. Pipette 100 μL of REF into a 20 mL volumetric flask and dilute |
| c RNI-d7 ≈ 5 ng/ml) | to volume with IS. |
| LOQ solution (LOQ) | 0.2 ng RNI/mL in IS (0.2 ng RNI/mL and approximately 5 ng |
| (c RNI = 0.2 ng/ml | RNI-d7/mL) |
| (equivalent to 80 ppb), | E.g. Pipette 400 μL of STD into a 10 mL volumetric flask and dilute |
| c RNI-d7 ≈ 5 ng/ml) | to volume with IS. |
| Note | weights and volumes can be adapted, as long as the |
| | concentration remains unchanged. |
| Evaluation/assessment | Determine the ratio of RNI peak area and RNI-d7 peak area in |
| | chromatograms of standard solution (STD) and sample solution. |
| | Reporting limit = 0.08 ppm |
| Calculation | Content, C, of RNI (ppm), based on the declared content |
| | $C = \frac{{R}_{S M P} \times {c}_{S S} \times {D}_{S M P} \times \overline{w}}{{R}_{S S} \times {w}_{S M P} \times A}$ |
| Where: | |
| R_{SMP} | Ratio of RNI peak area to RNI-d7 peak area in the |
| | chromatogram of test solution |
| R_{SS} | Ratio of RNI peak area to RNI-d7 peak area in the |
| | chromatogram of standard solution (STD) |
| w_{SMP} | Weight of sample [mg] |
| c_{SS} | Concentration of RNI in standard solution (= 5 ng/ml) |
| D_{SMP} | Dilution of test solution [mL] |
| w̅ | Average weight of tablet (= 136.05 mg) |
| A | Declared dose of LOU064 in the tablet (= 25 mg) |

Note:
in case of different test solution preparation (if validated), appropriate calculation needs to be applied.

### Example 16: Method for Validating the Content of Nitrite

### Chromatographic Condition:

### Nitrite by GRIESS Reaction and HPLC

Method HPLC UV:
Mobile Phase A: 0.1M Formic acid in water
Mobile Phase B: Acetonitrile
Analytical column: Water XBridge BEH C18, 100×3.0 mm, 2.5 um
Inj. Volume: 20 uL
Column Temperature: 40° C.
Flow rate: 0.8 mL/min
Needle wash: flush with Acetonitrile/Water 1:1 v/v
Detection: 548 nm
Gradient:

| Min | A(%) | B(%) |
|---|---|---|
| 0.0 | 90 | 10 |
| 1.5 | 90 | 10 |
| 6.0 | 20 | 80 |
| 6.5 | 20 | 80 |
| 6.6 | 90 | 10 |
| 10.0 | 90 | 10 |

### Samples Preparation for Sodium Carbonate Nitrite Content Determination:

### Sample Preparation of Sodium Carbonate:

Solvent: 85% o-H3PO4: water=37.5:62.5,
Neutralizing reagent: 8 mL Solvent+1 mL GRIESS reagent

Sample solution: Weigh the sample of about 600 mg sodium carbonate into a 10 mL volumetric flask, and add the neutralizing reagent (9 mL in total) in three times under the condition of water bath (carbon dioxide gas is generated during the addition process, the addition rate should control the carbon dioxide gas generation rate to ensure that bubbles do not overflow the capacity bottle, shake while adding). After the neutralization reagent is added, ultrasonic until there is no bubble (about 1-2 min), diluted with water to the volume.

### Sample Preparation of Comparison Standard:

Nitrite Std solution 1 mg/mL in Water & o-H3PO4 85%
Comparison stock solution: 0.1 mL Nitrite Std solution in 100 ml water=SSS (0.001 mg/mL)
Comparison solution: Transfer 60 μL comparison stock solution to 10 mL volumetric flask, add 8 mL solvent and 1 mL GRIESS reagent, dilute to volume with water, mix well (100 ppb).

### Samples Preparation for Sodium Hydroxide Nitrite Content Determination:

### Sample Preparation of Sodium Hydroxide:

Solvent: 85% o-H3PO4: water=37.5:62.5,
Sample stock solution: weigh 8000 mg NaOH in 10 mL volumetric flask, dilute to volume with MQ-Water, mix well. (Long-time ultrasound is prohibited, experimental data show that long-time ultrasonic glass products, NO2 will increase).
Sample solution: Transfer 1.5 mL of 85% o-H3PO4 and 0.5 mL GRIESS reagent into a 5 mL volumetric flask, put the volumetric flask into the ice water bath, transfer 2.5 ml sample stock solution, slowly drip into the 5 mL volumetric flask, shake the volumetric flask as much as possible, mix properly after the drip is completed, return to room temperature, and dilute with 85% o-H3PO4 to the volume. Mix well.

### Sample Preparation of Comparison Standard:

Nitrite Std solution 1 mg/mL in Water & o-H3PO4 85%
Nitrite Std solution 1 mg/ml in Water & o-H3PO4 85%
Comparison stock solution: 100 uL Nitrite Std solution in 100 ml water=SSS (0.001 mg/mL)
Comparison solution: Transfer 50 uL comparison stock solution to 5 mL volumetric flask, add 2 ml water and 0.5 mL GRIESS reagent, dilute to volume with 85% o-H3PO4, mix well (25 ppb).

1. 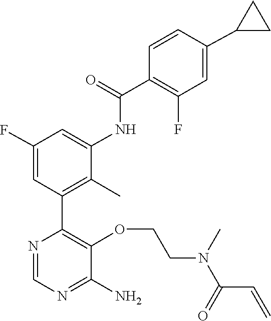 [SMILES: C=CC(=O)N(C)CCOc1c(N)ncnc1-c1cc(F)cc(NC(=O)c2ccc(C3CC3)cc2F)c1C]
(“LOU064”) drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide.

2. LOU064 drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, according to claim 1 wherein the content of said impurity is less than about 550 ppb, e.g. less than about 530 ppb.

3. LOU064 drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide according to claim 1 wherein the content of said impurity is less than about 400 ppb, e.g. less than about 360 ppb.

4. LOU064 drug substance substantially free of the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide according to claim 1 wherein the content of said impurity is less than about 150 ppb, e.g. less than about 130 ppb.

5. LOU064 drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, according to claim 1 wherein the content of said impurity is equal or less than about 100 ppb, e.g. less than about 90 ppb.

6-11. (canceled)

12. A pharmaceutical composition comprising LOU064 or a pharmaceutically acceptable salt thereof, wherein said composition is substantially free of nitrosamine impurities, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide.

13. (canceled)

14. The pharmaceutical composition of claim 12, wherein the total amount of nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, in the composition, is less than about 550 ppb, e.g. less than about 530 ppb, relative to the total amount of LOU064 in free form or in salt form.

15. The pharmaceutical composition of claim 12, wherein the total amount of nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, in the composition is less than about 400 ppb, e.g. less than about 360 ppb, relative to the total amount of LOU064 in free form or in salt form.

16. The pharmaceutical composition of claim 12, wherein the total amount of nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, in the composition, is less than about 150 ppb, e.g. less than about 130 ppb, relative to the total amount of LOU064 in free form or salt form.

17. The pharmaceutical composition of claim 12, wherein the total amount of nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, is equal or less than about 100 ppb, e.g. less than about 90 ppb, relative to the total amount of LOU064 in free form or salt form.

18-28. (canceled)

29. LOU064 drug substance or a pharmaceutical composition thereof, substantially free of nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide, wherein LOU064 drug substance is obtained by a process comprising:
a. providing a suspension comprising: 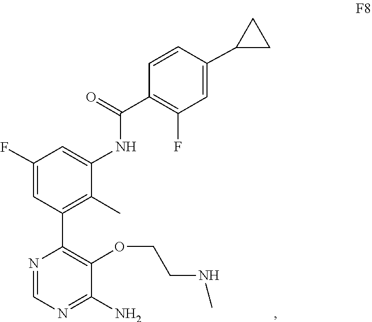 [SMILES: CNCCOc1c(N)ncnc1-c1cc(F)cc(NC(=O)c2ccc(C3CC3)cc2F)c1C]
a base, water and a solvent;
b. reacting the suspension with acrylic anhydride to provide LOU064 drug substance substantially free of a nitrosamine impurity, e.g. the nitrosamine impurity N-(3-(6-amino-5-(2(methyl)(nitroso)amino)ethoxy)pyrimidin-4-yl)-5-fluoro-2-methylphenyl)-4-cyclopropyl-2-fluorobenzamide.

30. LOU064 drug substance or a pharmaceutical composition comprising said drug substance, wherein LOU064 drug substance is prepared by the process according to claim 29, and wherein the base is selected from an inorganic base (e.g. Na₂CO₃, K₂CO₃, NaOH, KOH, Mg(OH)₂, Ca(OH)₂; e.g. Na₂CO₃).

31. LOU064 drug substance or a pharmaceutical composition comprising said drug substance, wherein LOU064 drug substance is prepared by the process according to claim 29, and wherein the base is a low nitrite content base, e.g. less than about 250 ppb or less than about 120 ppb.

32. LOU064 drug substance or a pharmaceutical composition comprising said drug substance, wherein LOU064 drug substance is prepared by the process according to claim 31, and wherein the low nitrite content sodium carbonate is Na₂CO₃·10H₂O, K₂CO₃·7H₂O, Na₂CO₃ with a low nitrite content or K₂CO₃ with a low nitrite content, e.g. a nitrite content of less than about 250 ppb.

33. LOU064 drug substance or a pharmaceutical composition comprising said drug substance, wherein LOU064 drug substance is prepared by the process of claim 29, and wherein the base is used in an amount between about 1.1 mole equivalent to about 1.2 equivalent mole equivalent.

34. LOU064 drug substance or a pharmaceutical composition comprising said drug substance, wherein LOU064 drug substance is prepared by the process of claim 29, and wherein the solvent is selected from MeTHF, THF, alcohols (isopropanol), dichloromethane, toluene, ethyl acetate, isopropyl acetate, acetonitrile, acetone, tert-butylmethylether (TBME), e.g. ethyl acetate, optionally wherein the solvent has a low nitrite content (e.g. less than 1.5 ppm, less than 1 ppm, less than 0.5 ppm, less than 0.2 ppm).

35. LOU064 drug substance or a pharmaceutical composition comprising said drug substance, wherein LOU064 drug substance is prepared by the process of claim 29, and wherein the suspension in a) comprises at least about 25 mole equivalents of water, optionally purified water.

36-38. (canceled)

39. LOU064 drug substance or a pharmaceutical composition comprising said drug substance, wherein LOU064 drug substance is prepared by the process of claim 29, and wherein acrylic anhydride is added as a solution in a solvent (e.g. in ethyl acetate) to the suspension of a).

40. LOU064 drug substance or a pharmaceutical composition comprising said drug substance, wherein LOU064 drug substance is prepared by process of claim 29, further comprising the step of deprotecting F7 to provide F8: 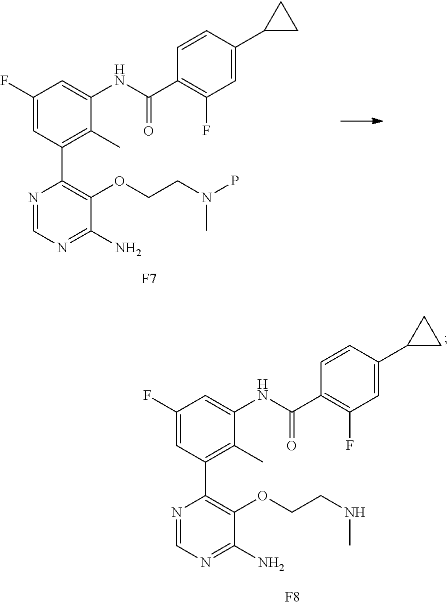
wherein P is an amino protective group, e.g. tert-butyloxycarbonyl (BOC).

41. LOU064 drug substance or a pharmaceutical composition comprising said drug substance, wherein LOU064 drug substance is prepared by the process according to claim 40, wherein F7 is deprotected in the presence of an acid (e.g. HCl) and F8 is isolated after a neutralization step, e.g. neutralization with a base, e.g. with a low nitrite content NaOH (e.g. a solution of low nitrite content NaOH in purified water).

42. LOU064 drug substance or a pharmaceutical composition comprising said drug substance, wherein LOU064 drug substance is prepared by the process according to claim 40, and wherein F8 is added into step of a) without any drying step.

43-55. (canceled)
